(19) United States
(12) Patent Application Publication
(10) Pub. No.: US 2023/0225823 A1
(43) Pub. Date: Jul. 20, 2023

(54) SYSTEMS AND METHODS FOR CONTROLLING THE SPREAD OF AIRBORNE MATERIALS DURING CLINICAL OR LABORATORY PROCEDURES

(71) Applicant: Massachusetts Eye and Ear Infirmary, Boston, MA (US)

(72) Inventors: Alicia M. Quesnel, Waban, MA (US); Aaron Remenschneider, Boston, MA (US); Elliot D. Kozin, Boston, MA (US); Daniel J. Lee, Brookline, MA (US); Jenny X. Chen, Boston, MA (US); Divya A. Chari, Boston, MA (US)

(21) Appl. No.: 17/996,715

(22) PCT Filed: Apr. 20, 2021
(86) PCT No.: PCT/US2021/028241

§ 371 Date: Oct. 20, 2022

Related U.S. Application Data
(60) Provisional application No. 63/012,729, filed on Apr. 20, 2020.
(60) Provisional application No. 63/023,615, filed on May 12, 2020.
(60) Provisional application No. 63/017,551, filed on Apr. 29, 2020.
(60) Provisional application No. 63/028,840, filed on May 22, 2020.

(51) Int. Cl. A61B 90/40; A61B 90/00

(52) U.S. Cl. CPC A61B 90/40; A61B 90/05; A61B 2090/401

(57) ABSTRACT

A system can include a barrier configured to form a three-dimensional (3D) structure to define an interior volume and an exterior space and to mitigate fluid movement from the interior volume and into the exterior space thereby mitigating droplet, articulate, and aerosol movement in the flow path of the fluid from the interior volume and into the exterior space. The system can include a plurality of passages extending through the barrier to provide access from the exterior space and into the interior volume to perform the procedure on the patient or perform the study on the laboratory sample arranged in the interior volume. The barrier can be configured to be formed in the 3D structure to arrange a portion of the patient or the laboratory sample in the interior volume. The barrier can be external to the patient.

### CROSS-REFERENCE TO RELATED APPLICATIONS

This application claims priority to U.S. Patent Application No. 63/012,729 filed Apr. 20, 2020, and entitled, “Surgical Shields for Reduction of Particulate and Aerosolized Debris,” which is hereby incorporated by reference in its entirety. This application claims priority to U.S. Patent Application No. 63/023,615 filed May 12, 2020, and entitled, “Systems and Methods for Controlling the Spread of Airborne Materials during Clinical or Laboratory Procedures,” which is hereby incorporated by reference in its entirety. This application claims priority to U.S. Patent Application No. 63/017,551 filed Apr. 29, 2020, and entitled, “Universally Adaptable Three Dimensional Surgical Shield for Reduction of Particle and Aerosol Debris,” which is hereby incorporated by reference in its entirety. This application claims priority to U.S. Patent Application No. 63/028,840 filed May 22, 2020, and entitled, “Systems and Methods for Controlling the Spread of Airborne Materials during Clinical or Laboratory Procedures,” which is hereby incorporated by reference in its entirety.

### STATEMENT REGARDING FEDERALLY SPONSORED RESEARCH

Not applicable.

### BACKGROUND

Typically, surgical procedures aim to prevent materials, including pathogens, from entering the surgical site (e.g., a surgical field) of a patient. This is often accomplished by utilizing sterile drapes that cover areas surrounding the surgical site, and specialized clothing worn by personnel (e.g., gloves scrubs, gowns, masks, etc.) all of which prevent the transmission of pathogens from the exterior environment and into the surgical site of the patient. However, while these practices often mitigate transmission of pathogens into a patient, these current practices can be inadequate to protect the personnel from the transmission of materials, such as pathogens, from the patient. Thus, it would be desirable to have improved systems and methods for controlling spread of aerosolized substances (including airborne liquids and particulates, and droplets) during clinical or laboratory procedures.

### SUMMARY OF THE DISCLOSURE

The present disclosure provides systems and methods that overcome the aforementioned drawbacks by controlling or mitigating the transmission of materials, including pathogens, from an enclosed area surrounding the patient or a laboratory sample into an exterior area occupied by personnel. As will be described, systems and methods are provided to form such as enclosure using a barrier configured to form a three-dimensional (3D) structure. The 3D structure may include passages extending through the barrier to provide access from the exterior space and into the interior volume. Such passages may have further systems to control materials from exiting the interior space via the passages. The 3D structure may also include systems to restrict movement form the interior space to the exterior along joints, folds, or edges of the barrier. Thus, a new concept in clinical tool is provided that serves to control and mitigate the transmission of materials, including pathogens, from an interior space that encloses the patient or laboratory sample that is the focus of the clinical or laboratory procedure into an exterior area occupied by one of the personnel. This new paradigm seeks to not just protect the patient or laboratory sample from materials, including pathogens, in the exterior world, but to also protect the personnel form the materials, including pathogens, present with the patient or laboratory sample when performing a clinical procedure.

In some aspects of the disclosure, systems and methods are provided for controlling the spread of airborne substances during clinical or laboratory procedures. Some non-limiting examples of the disclosure provide a system for enclosing and separating one of a patient from personnel while the personnel perform a procedure on the patient, or a laboratory sample from a technician while studying the laboratory sample, the system comprising: a barrier configured to form a three-dimensional (3D) structure to define an interior volume and an exterior space and to mitigate fluid movement from the interior volume and into the exterior space thereby mitigating droplet, particulate, and aerosol movement in the flow path of the fluid from the interior volume and into the exterior space; a plurality of passages extending through the barrier to provide access from the exterior space and into the interior volume to perform the procedure on the patient or perform the study on the laboratory sample arranged in the interior volume, and the barrier is configured to be formed in the 3D structure to arrange a portion of the patient or the laboratory sample in the interior volume. The barrier can be positioned to be external to the patient if the personnel perform a procedure on the patient. In some non-limiting examples, the entire barrier can be positioned external to the patient.

In some aspects, a free end of the barrier includes a flange that extends along and engages with a portion of a patient or the laboratory sample or a structure to enclose the patient or the laboratory sample.

In some aspects, the flange includes a coupling layer that couples to the portion of a patient or the laboratory sample.

In some aspects, the flange has a first surface and a second surface. The coupling layer can be coupled to the first surface of the barrier.

In some aspects, the coupling layer includes an adhesive.

In some aspects, the isolation system can include a support structure that maintains at least a portion of the 3D structure.

In some aspects, the support structure can include a scaffold that is coupled to an interior volume of the barrier, coupled to the exterior surface of the barrier, or integrated within the barrier.

In some aspects, the scaffold includes a plurality of support beams.

In some aspects, a first support beam within the plurality of support beams is curved.

In some aspects, two adjacent support beams within the plurality of support beams are separated along an axial axis of the 3D structure.

In some aspects, a given support beam within the plurality of support beams is coupled to the two adjacent support beams.

In some aspects, the given support beam includes a resilient portion, the resilient portion configured to bend to allow the given support beam to be folded or shaped.

In some aspects, the support structure can include a sleeve coupled to at least one of the interior surface and the exterior surface of the barrier, the sleeve configured to receive a support beam.

In some aspects, the barrier can include a central region, a first region with a first free end extending from the central region, the first region having a first edge, and a second region with a second free end extending from the central region, the second region having a second edge. The first edge of the first region can be configured to be coupled to the second edge of the second region to join the first region to the second region. The coupling of the first edge to the second edge provides an adjoined edge of the barrier that mitigates fluid movement from the interior volume and into the exterior space along the adjoined edge thereby mitigating droplet, particulate, and aerosol movement from the interior volume and interior the exterior space along the adjoined edge.

In some aspects, the first edge of the first region includes a strip that extends along a portion of the first edge.

In some aspects, the strip includes and an adhesive layer configured to be secured to a surface of the second region.

In some aspects, the strip is removable coupled to a surface of the second region.

In some aspects, the strip includes at least one of a hook and a loop fastener, and the surface of the second region includes the other of the at least one of the hook and the loop fastener.

In some aspects, the first edge of the first region includes a first sleeve that extends along a portion of the first edge. The second edge of the second region can include a second sleeve that extends along a portion of the first edge. The scaffolding can include a support beam that is received though the first sleeve and though the second sleeve to couple the first region to the second region.

In some aspects, the first sleeve is positioned above the second sleeve.

In some aspects, a portion of the first sleeve and a portion of the second sleeve extend along the same axial dimension of an axial axis of the 3D structure.

In some aspects, the barrier includes the creases. The support structure can include a support layer coupled to and situated within a portion of the crease. The support layer can be configured to maintain the shape of the crease.

In some aspects, the barrier can include the creases. The creases can include a first crease being curved, and a second crease being curved. The first crease, and the second crease can be axially displaced from each other along an axial axis of the 3D structure.

In some aspects, the first crease and the second crease are coaxially aligned with each other along the axial axis.

In some aspects, the barrier includes the creases, and the creases can include a first crease being curved and extending along an axial axis of the 3D structure, and a second crease being curved and extending along the axial axis of the 3D structure. The first crease, and the second crease can emanate from the axial axis along opposing directions.

In some aspects, the barrier can include a tab emanating from an exterior surface of the barrier. The tab can have a first surface that is positioned towards the patient or the laboratory sample, and an opposite second surface that is positioned towards the exterior space. The first surface of the tab can have a coupling layer configured to be secured to a structure to couple the barrier to the structure.

In some aspects, the coupling layer includes an adhesive layer.

In some aspects, the coupling layer removably couples the tab to the structure.

In some aspects, the coupling layer is at least one of a hook and a loop fastener.

In some aspects, the barrier can include a plurality of tabs emanating from the exterior surface of the barrier. The plurality of tabs can have a coupling layer. The plurality of tabs can include the tab.

In some aspects, the barrier has a thickness. The thickness of the barrier can increase along a portion of the barrier.

In some aspects, the 3D structure has a first axial end located proximal to the patient or the laboratory sample and a second axial end opposite the first axial end. The first axial end and the second axial end can be positioned along an axial axis of the 3D structure. The portion of the barrier can have the increased thickness can be positioned closer to the first axial end than the second axial end. The portion of the barrier can have the increased thickness maintains the 3D structure of the barrier.

In some aspects, the isolation system can include a weight that is coupled to an interior surface of the barrier, coupled to the exterior surface of the barrier, or integrated within the barrier.

In some aspects, the 3D structure has a first axial end located proximal to the patient or the laboratory sample and a second axial end opposite the first axial end. The first axial end and the second axial end can be positioned along an axial axis of the 3D structure. The weight can be coupled to or integrated within the barrier at a location that is closer to the first axial end than the second axial end. The weight can maintain the 3D structure of the barrier.

In some aspects, a free end of the barrier can include a flange that extends along and engages with a portion of a patient or the laboratory sample or a structure to enclose the patient or the laboratory sample. The flange can have a first surface and a second surface. The first surface can have a coupling layer that couples to the portion of a patient or the laboratory sample or the structure. The weight can be coupled to the second surface of the flange.

In some aspects, the weight extends along a portion of the flange to reinforce the structure of the flange to maintain the shape of the 3D structure.

In some aspects, the plurality of passages include an arm port configured to receive an arm of one of the personnel.

In some aspects, the arm port includes an aperture.

In some aspects, the isolation system can include a flap that is removably coupled to the barrier. When the flap is coupled to the barrier, the flap can extend across the aperture to generate a seal.

In some aspects, the flap is magnetically coupled to the barrier.

In some aspects, the flap includes at least one of a hook and a loop fastener, and the barrier includes the other of the at least one of the hook and the loop fastener.

In some aspects, the arm port can be a valve that prevents fluid contained in the interior volume from flowing through the valve and into the exterior space. When the arm is inserted through the valve an into the interior volume, the valve can sealingly engage with the arm to prevent fluid contained in the interior volume from flowing through the valve and into the exterior space when the arm is inserted into the valve.

In some aspects, the valve includes a plurality of flaps that contact each other to generate a seal.

In some aspects, the isolation system can include a rigid attachment having a hole therethrough that interfaces with and is coupled to the boundary that defines the aperture.

In some aspects, the isolation system can include a plug removably coupled to the rigid attachment that generates a seal to prevent fluid in the interior volume from flowing through the hole of the rigid attachment and into the exterior space.

In some aspects, the plug is threadingly engaged with the rigid attachment.

In some aspects, an arm sleeve having a rigid cuff is removably coupled to the rigid attachment.

In some aspects, the isolation system can include a fastening assembly including a fastener and a strip of material. The arm sleeve can be configured to be rolled into a compact configuration. The strip of material can be wrapped around the arm sleeve in the compact configuration and fastened with the fastener.

In some aspects, the arm sleeve can include a proximal portion and a distal portion. The proximal end and the distal end can be removably coupled from each other.

In some aspects, the arm sleeve includes a clasp locker having a slider that when slid either couples or decouples the proximal portion to or from the distal end of the arm sleeve.

In some aspects, when the arm sleeve is in a first configuration the slide is positioned internally to the arm sleeve and in the exterior space. When the arm sleeve is inverted to a second configuration the slide is positioned externally to the arm sleeve and within the interior volume of the 3D structure.

In some aspects, the arm sleeve includes a proximal end and an opposite distal end. The arm sleeve can include a region of material weakness that is circumferential.

In some aspects, the region of material weakness includes a perforation.

In some aspects, the isolation system can include an arm sleeve integrally formed with the barrier. The arm sleeve can extend away from the barrier. The sleeve can be configured to be inverted when inserted into the interior volume of the 3D structure.

In some aspects, a given passage of the plurality of passages includes an instrument attachment configured to be removably coupled to an instrument.

In some aspects, the instrument is a magnifying instrument. The magnifying instrument can be at least one of a microscope, an endoscope, and an exoscope.

In some aspects, the instrument attachment can include an elastic ring that is seated within the given passage, or is coupled to the exterior surface or the interior surface of the barrier proximal to the given passage. The elastic ring is configured to expand to surround a portion of the instrument to secure the barrier to the instrument and to generate a seal that prevents fluid contained within the interior volume from flowing through the given passage and into the exterior space.

In some aspects, the instrument attachment includes a cuff extending from the exterior surface of the barrier and surrounding a portion of the given passage, the cuff having a free end that is configured to be coupled to the cuff to adjust a circumference of the cuff to couple the cuff to a portion of the instrument.

In some aspects, a given passage of the plurality of passages includes a first fluid port.

In some aspects, the isolation system can include a first filter in sealing engagement with the first fluid port The first filter can allow fluid contained in the interior volume to flow out through the first filter and into the exterior space, and fluid within the exterior space to flow through the first filter and into the exterior space.

In some aspects, the first filter is at least one of positioned within the first fluid port, or extended across the first fluid port.

In some aspects, the first filter has pores sized to block at least one of pathogens, and tissue particulates.

In some aspects, another given passage of the plurality of passages includes a second fluid port. The isolation system can include a second filter in sealing engagement with the second fluid port. The second filter can allow fluid contained in the interior volume to flow out through the second filter and into the exterior space, and fluid within the exterior space to flow through the second filter and into the interior volume. The first port can have the first filter, and the second port can have the second filter allowing the volume of the interior volume to be substantially constant.

In some aspects, the first fluid port is sized to receive a suction probe of a suction system.

In some aspects, the isolation system can include a suction attachment. The suction probe can be removably coupled to the first fluid port.

In some aspects, the suction attachment can be a valve. The valve being configured to sealingly engage the probe of the suction system when the probe is inserted through the valve and into the interior volume.

In some aspects, the first fluid port includes a rigid attachment. The suction probe can be configured to be threadingly engage with the rigid attachment to provide a sealing engagement between the first fluid port and the suction probe.

In some aspects, the 3D structure has a proximal side, a distal side, and adjacent sides between the proximal and distal sides. The proximal side can be positioned towards one of the personnel. The plurality of passages can include a first arm port, and a second arm port located on the proximal side of the 3D structure. The first fluid port can be positioned on the proximal side of the 3D structure.

In some aspects, the isolation system can include a bag extending from the exterior surface of the barrier. The bag can be in fluid communication with the interior volume of the 3D structure. The first fluid port can be located on the bag.

In some aspects, the barrier allows all wavelengths of visible light to pass through.

In some aspects, the barrier has a visible light attenuation coefficient in a range.

In some aspects, the isolation system can include a window integrated within a portion of the barrier. The window can be formed of a material that allows a visible wavelength of light to pass through.

In some aspects, a portion of an interior surface of the barrier has a coating that captures at least one of pathogens, and tissue particulates.

In some aspects, the coating is charged to attract pathogens and tissue particulates having the opposing charge, and to repel pathogens and tissue particulates having a similar charge.

In some aspects, the coating is an adhesive.

In some aspects, the coating includes at least one of a disinfectant, a medicant, or a binding agent. The at least one of the disinfectant, the medicant, or the binding agent inactivate the pathogen.

In some aspects, the isolation system can include a debris detection system positioned within the interior volume of the 3D structure.

In some aspects, the debris detection system can include a light source, an image sensor, and a processor. The processor can be configured to acquire imaging data from the image sensor, and quantify a density of particles within the interior volume from the imaging data.

In some aspects, the isolation system can include a suction probe positioned within the interior volume of the 3D structure. The processor can be further configured to compare the density of the particles to a threshold density, and activate the suction probe to provide suction based on the density of the particles exceeding the threshold density.

In some aspects, the barrier includes a series of pleats. The series of pleats can be circumferentially oriented around an axial axis of the 3D structure.

In some aspects, the pleats are at least one of: a knife pleat, a box pleat, a double box pleat, or a cartridge pleat.

In some aspects, the barrier includes a series of creases. The series of creases are curved and are oriented along an axial axis of the 3D structure, or are circumferentially positioned and separated along the axial axis.

In other aspects of the disclosure, a system is provided for enclosing and separating a patient from personnel while the personnel perform a procedure on the patient, the system comprising: a barrier configured to form a three-dimensional (3D) structure to define an interior volume and an exterior space and to mitigate fluid movement from the interior volume and into the exterior space thereby mitigating droplet, particulate, and aerosol movement in the flow path of the fluid from the interior volume and into the exterior space; a plurality of passages extending through the barrier to provide access from the exterior space and into the interior volume to perform the clinical procedure on the patient, and the barrier is configured to be formed in the 3D structure to arrange a portion of the patient in the interior volume. The barrier can be positioned external to the patient. In some non-limiting examples, the entire barrier can be positioned external to the patient.

In still other aspects of the disclosure, a system is provided for enclosing a magnifying instrument, the system comprising: a barrier configured to form a three-dimensional (3D) structure to define an interior volume and an exterior space and to mitigate fluid movement from the interior volume and into the exterior space thereby mitigating droplet, particulate, and aerosol movement in the flow path from the interior volume and into the exterior space; a plurality of passages extending through the barrier to provide access from the exterior space and into the interior volume, wherein the barrier is configured to be formed in the 3D structure to arrange a portion of a patient or a laboratory sample in the interior volume, and wherein a field of view of the magnifying instrument is located within the interior volume of the barrier.

Other aspects and features of the disclosure are provided throughout.

### BRIEF DESCRIPTIONS OF THE DRAWINGS

FIG. 1 shows a schematic illustration of an example of an isolation system.

FIG. 2 shows a schematic illustration of an example of a sanitation system.

FIG. 3 shows a side perspective view of another isolation system.

FIG. 4 shows a top view of the isolation system of FIG. 3.

FIG. 5 shows a side perspective view of another isolation system.

FIG. 6 shows a top view of the isolation system of FIG. 5.

FIG. 7 shows a perspective view of another isolation system.

FIG. 8 a perspective view of the isolation system of FIG. 7 in a compressed state.

FIG. 9 shows a cross-sectional view of a support beam of the isolation system of FIG. 8, taken along line 9-9 of FIG. 8.

FIG. 10 is a top view of another isolations system in a deconstructed two-dimensional (“2D”) configuration.

FIG. 11 is a perspective view of the isolation system of FIG. 10 in a constructed three-dimensional (“3D”) configuration.

FIG. 12 shows a perspective view of free ends of the isolation system of FIG. 10 being coupled together.

FIG. 13 shows a perspective view of free ends of the isolation system of FIG. 10 being coupled together with a support beam.

FIG. 14 shows a perspective view of another barrier having free ends being coupled together.

FIG. 15 shows a top view of another isolation system.

FIG. 16 shows a front view of the isolation system of FIG. 15 in a folded state.

FIG. 17 shows a support layer coupled to a fold of the isolation system of FIG. 15.

FIG. 18 shows an example of a pleat that can be used to form a 3D structure of an isolation system.

FIG. 19 shows an example of another pleat that can be used to form a 3D structure of an isolation system.

FIG. 20 shows a perspective view of another isolation system.

FIG. 21 shows a side cross-sectional view of the isolation system of FIG. 20, taken along line 21-21 of FIG. 20.

FIG. 22 shows a top view of an example of an arm port.

FIG. 23 shows a top view of an example of another arm port.

FIG. 24 shows a perspective view of an example of another arm port.

FIG. 25 shows a top view of an example of another arm port.

FIG. 26 shows a partial cross-sectional view of the arm port of FIG. 25, taken along line 26-26 of FIG. 25.

FIG. 27 shows a side view of an example of a plug.

FIG. 28 shows a top view of an example of an arm sleeve in a first configuration.

FIG. 29 shows a top view of the arm sleeve of FIG. 28, in a second configuration.

FIG. 30 shows a top view of another arm sleeve.

FIG. 31 shows a top view of an instrument attachment.

FIG. 32 shows a side view of the instrument attachment of FIG. 31.

FIG. 33 shows another example of an instrument attachment.

FIG. 34 shows an example of a viewing window for an isolation system.

FIG. 35 shows a perspective view of a fluid port.

FIG. 36 shows a side view of the fluid port of FIG. 35.

FIG. 37 shows an example of a schematic implementation of an isolation system having fluid ports of FIG. 36 and a suction source.

FIG. 38 shows a perspective view of a suction port.

FIG. 39 shows a perspective view of a suction port of FIG. 38 prior to the insertion of a suction probe.

FIG. 40 shows a perspective view of the suction port of FIG. 38 after the insertion of the suction probe.

FIG. 41 shows an example of another isolation system.

FIG. 42 shows an example of another isolation system.

FIG. 43 shows a side cross-sectional view of the isolation system of FIG. 42, taken along line 43-43 of FIG. 42.

FIG. 44 shows a front view of another isolation system.

FIG. 45 shows a front perspective view of another isolation system.

FIG. 46 shows a front view of another isolation system.

FIG. 47 shows a top view of the isolation system of FIG. 46.

FIG. 48A shows a front view of another isolation system.

FIG. 48B shows a front view of another isolation system.

FIG. 48C shows a front view of another isolation system.

FIG. 48D shows a front view of another isolation system.

FIG. 49 shows a front view of another isolation system.

FIG. 50 shows a flowchart of a process for preparing a patient for a procedure, or a sample for observation.

FIG. 51A shows the aerosolization of fluorescent bone dust and droplets occurring during a mastoidectomy.

FIG. 51B shows fluorescent debris (some indicated by arrowheads) soiling a surgeon's chest, arms, and lap under ultraviolet light after drilling part of a cortical mastoidectomy for 2 minutes, with size 6 cutting bur, at 70,000 RPM.

FIG. 51C shows an image illustrating fluorescent particulate matter scattered on the surgeon's face shield and hair covering (shown with arrowheads) after 2 minutes of drilling.

FIG. 52A shows an octagonal grid to define distances and locations of particulate debris from the ear canal.

FIG. 52B shows a sample of an aerial photo of grid under ultraviolet light with the cadaveric specimen marked by the “X”.

FIG. 52C shows a sample of a close up photo of one segment of the grid with numerous fine fluorescent particles, representing bone dust or fluorescein stained droplets.

FIG. 53A shows the 1060 Steri-drape that was used to create a barrier drape that enclosed the microscope lens, cadaveric head specimen, and immediate surrounding 30 cm surgical field (referred to as the “OtoTent”).

FIG. 53B shows a sketch of how a hole is cut in the adhesive portion of the drape to allow for the microscope lens.

FIG. 53C shows a photo of the OtoTent in position on the microscope, with the edge of the drape lifted.

FIG. 54A shows the positioning and use of the OtoTent.

FIG. 54B shows an image of surgeon operating under the OtoTent.

FIG. 54C shows an image of the underside of the OtoTent after drilling for 60 seconds.

FIG. 55A shows a simulation without the microscope where there is a predominance of particles in the quadrants closest to the surgeon.

FIG. 55B shows that the addition of the microscope still results in particulate dispersion that is highest in quadrants 1 and 2 (adjacent to the surgeon), and also still demonstrates particulate dispersion away from the surgeon (potentially toward other operating room staff).

FIG. 55C shows a simulation with the OtoTent (dotted lines) where there is decreased particulate matter in all areas, including both the inner and outer octagons.

FIG. 56 shows a table for percent surface density (“PSD”) and percent surface area % SA) covered in particulate after drilling in each test condition. SD=standard deviation.

FIGS. 57A shows a show graph that quantifies fluorescent particles under three test conditions: no microscope, microscope, and microscope+OtoTent in terms of particle surface density.

FIGS. 57B shows a show graph that quantifies fluorescent particles under three test conditions: no microscope, microscope, and microscope+OtoTent in terms of percent (%) surface area covered by particles.

FIGS. 57C shows a show graph that quantifies fluorescent particles under three test conditions: no microscope, microscope, and microscope+OtoTent in terms of particle surface density as a function of distance from the external auditor canal (“EAC”).

FIGS. 57D shows a show graph that quantifies fluorescent particles under three test conditions: no microscope, microscope, and microscope+OtoTent in terms of percent (%) surface area covered by particles as a function of distance from the EAC.

FIG. 58A shows a configuration that has no barrier during a drilling condition with the use of the Zeiss microscope.

FIG. 58B shows a configuration that utilizes an OtoTent barrier.

FIG. 58C shows a configuration that utilizes an OtoShield barrier with a built-in arm ports, enclosed floor, instrument/suction ports, and collapsible frame.

FIG. 59 shows an image of a cadaveric head specimen with a second suction attached to the cadaveric head.

FIG. 60 shows a graph of the maximum number of particles generated during 10 second increments with a flow rate of 1 L/min across particle size for mastoidectomy without a barrier in the following conditions: cutting bur, cutting bur with second suction, cutting bur with no suction, cutting bur with suction irrigator, and diamond bur.

FIG. 61 shows a graph of the average particle density across time for mastoidectomy without a barrier in two conditions: cutting bur and cutting bur with a second suction. Drilling occurred between time points 60-120 seconds.

FIG. 62 shows a graph comparing the particle density generated in mastoidectomy without a barrier and with the OtoTent and OtoShield, across time, and with and without a second suction.

FIG. 63A shows a graph of the particle density generated during one minute of drilling for Mastoidectomy without a barrier drape, mastoidectomy without a barrier drape but with second suction, and OtoTent without second suction that showed high rates of particle generation during drilling.

FIG. 63B shows another graph of the particle density generated following barrier removal either immediately or after one minute had elapsed after drilling.

### DETAILED DESCRIPTION

Current clinical practices aim to prevent the external environment (e.g., the operating room) from contaminating the surgical site of the patient. In other words, the general aim is to isolate the patient or the surgical site from the external environment, except carefully-selected and sterilized tools or resources. However, the recent emergence of patients that (potentially or that do) harbor the novel severe acute respiratory syndrome coronavirus 2 (“SARS-CoV-19” or COVID-19) in the hospital setting has revealed a gap in current operatory practices in protecting personnel from materials or pathogens associated with the patient or a laboratory sample. For example, while sterilized gowns, masks, scrubs, etc., may decrease the transmission of the virus (or other pathogens) from the patient to personnel, these protective measures are still far from perfect. In fact, these measures can be severely ineffective during procedures that generate aerosols, or other airborne tissue particulates, which are known to more effectively transmit certain pathogens (e.g., the SARS-CoV-19). These measures are even worse when the aerosolizing procedure is conducted at a location where the pathogen is known to reside.

Some non-limiting examples of the disclosure provide systems and methods for controlling the spread of airborne substances during clinical or laboratory procedures, which provide improvements over typical isolation systems. For example, some non-limiting examples of the disclosure provide a barrier that forms a three-dimensional (“3D”) structure that encloses a patient during an operatory procedure. The barrier having the 3D structure has an internal volume that encloses a surgical site of the patient, and thus mitigates fluid movement (that can contain pathogen laden aerosols, tissue particulates, etc.) from escaping into the exterior space (e.g., the operatory room). This isolation of the personnel from the potentially infected patient provides a unique change in current sanitation philosophy aimed at allowing the personnel to effectively complete a procedure, while simultaneously ensuring that the personnel are adequately protected from pathogen transmission. In some cases, this approach can be particularly helpful during clinical or laboratory procedures that produce aerosolized debris (e.g., tissue, bone, fluids such as mucosa, etc.), because some aerosolized debris (e.g., tissue) pose a greater risk to contraction of a disease as compared to other particulates (e.g., small droplets that contain the infectious agent, such as bacteria, fungi, and viruses) that are not produced during these types of procedures (e.g., aerosol producing procedures).

Some non-limiting examples of the disclosure provide components (or features) that help to effectively facilitate the procedure, or which aid in the removal (or identification) of aerosols or particulates that can be pathogen laden. For example, the barrier can include components (or can have properties) that can help to maintain the 3D structure, such as, for example, a scaffold, pleats, creases (having support layers), a folding pattern, tabs, flanges, weights, instrument attachments (e.g., for magnifying instruments, such as microscopes, endoscopes, exoscopes, etc.), etc. As another example, the barrier can include components (or features) that can help to effectively facilitate the procedure, such as, for example, passages including instruments ports, arm ports, viewing windows, suction ports, etc. These passages that extend through the barrier can provide access from the exterior space and into the interior volume of the barrier. Thus, passages extending through the barrier, can be passages that are through the material of the barrier (e.g., holes, apertures, etc.), and can be passages beneath (or underneath) the barrier (e.g., to form a non-hermetic seal, acting as a curtain over an arm of one of the personnel). As yet another example, the barrier can include components or features that can help to remove aerosols or particulates, such as, for example, suction bags, suction ports, filters, barrier coatings (or barrier properties), etc. As still another example, the barrier can include components that can help to identify (and quantify) the amount of particulates or aerosols in the interior volume defined by the 3D structure of the barrier.

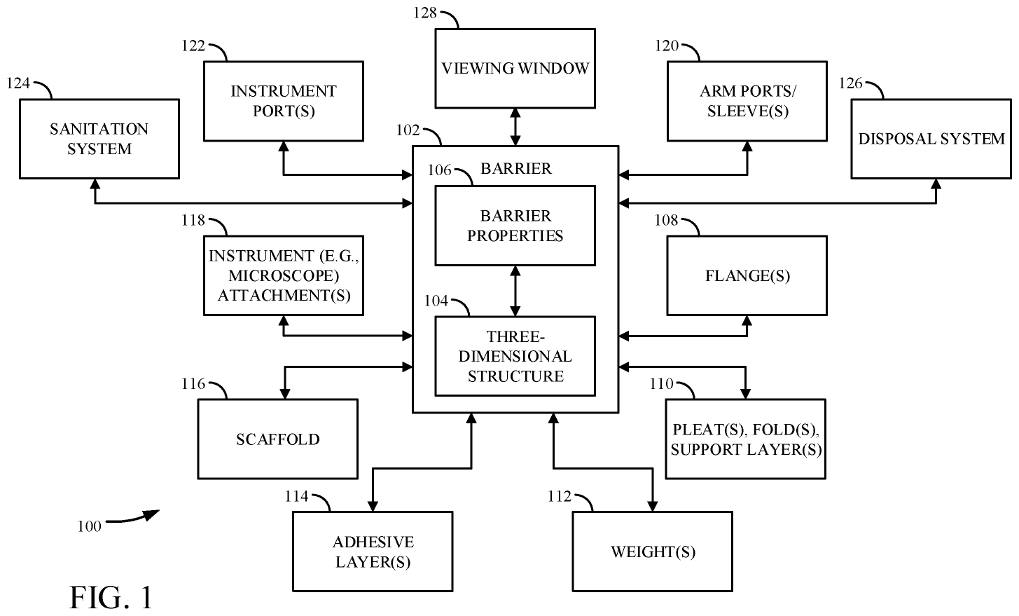

FIG. 1 shows a schematic illustration of an example of an isolation system 100 enclosing a patient or a sample. The isolation system 100 can include a barrier 102 having a three-dimensional structure 104 and barrier properties 106, flanges 108, pleats, folds, support layers 110, weights 112, adhesives 114, a scaffold 116, and instrument attachments 118. The barrier 102 can have an interior surface and an opposite exterior surface, where the interior surface faces (and is in fluid communication with) the interior volume of the barrier 102, and where the exterior surface faces (and is in fluid communication with) the exterior space (e.g., the operating room). The barrier 102 can be formed of a single layer (or otherwise a single sheet), multiple layers (e.g., two sheets), etc. The barrier 102 can be out of various materials, such as those materials utilized in surgical drapes. For example, in some non-limiting examples, the barrier 102 can be formed out of fabrics, plastics (e.g., polyethylene), etc.

In some non-limiting examples, materials of the barrier 102 can include metals (e.g., metal alloys), polymers, plastics (e.g., pressure or heat activated thermoplastics, polylactic acid plastics, polycaprolactone plastics, polyvinyl chloride plastics), silicones, glass, fiberglass, ceramics, mesh, fabric, etc. In some cases, at least one material of the barrier 102 at an appropriate portion of the barrier 102 can be partially (or fully) transparent so as to allow the personnel to view the contents of the interior volume of the 3D structure (e.g., when conducting the procedure). In some non-limiting examples, the materials of the barrier 102 can be flexible while being rigid (e.g., to define the 3D structure). For example, the barrier 102 can have a degree of flexibility and a degree of rigidity. In some non-limiting examples, the barrier 102 can have an outer surface (or inner surface) that has antireflective properties (or includes an antireflective coating), so as to reduce glare (e.g., from operating room lights).

The barrier 102 is configured to be formed into the 3D structure 104 that defines an interior volume and separates the interior volume from the exterior space. In other words, the 3D structure 104 isolates (or otherwise mitigates fluid movement) from the internal volume and into the exterior space (or vice versa). The 3D structure 104 can embody many different shapes, such as, for example, a hemisphere, a cone, a frustoconical structure, pyramidal structures (e.g., square pyramids, triangular pyramids, etc.), prisms (e.g., octagonal prisms, rectangular prisms, square prisms, etc.). In some non-limiting examples, the barrier properties 106 of the barrier 102 can help to maintain the 3D structure 104. For example, the barrier properties 106 can include a varying thickness of the barrier 102 that distributes the weight of the barrier at particular locations so as to maintain the 3D structure 104. As a more specific example, the thickness of the barrier 102 can be increased near an end of the barrier 102 that contacts the patient. This way, the inherent increased weight of the barrier 102 pulls other portions of the barrier 102 to maintain the 3D structure 104 (e.g., pulls other portions of the barrier 102 taut). In some cases, the increased thickness of the barrier 102 can be located along circumferential portions (or otherwise along portions of the perimeter) of the barrier 102. As another example, the increased thicknesses of the barrier 102 can be located near on or at opposing sides of creases, pleats, or other supporting structures such as a support beam of a scaffold. In this way, with the increased weight on opposing sides, the increased weight can further define these features or otherwise prevent changing of the 3D structure 104.

In some non-limiting examples, the barrier 102 can include flanges 108 that can also help to maintain the 3D structure 104 or facilitate maintaining a complete enclosure, without undesired gaps or passages, such as along edges of the barrier 102 (e.g., where the barrier 102 meets the floor or two walls of the barrier 102 meet). In some non-limiting examples, the flanges 108 are extensions of the barrier 102, but which are formed when the three-dimensional structure 104 is formed. In this case, for example, the flanges 108 can have a coupling layer (e.g., an adhesive layer) disposed on the exterior surface of the flange 108 (e.g., in a pre-formed or flat configuration) that engages with the patient. When this coupling layer is engaged with a subject, the barrier 102 bends to form the flange 108. Thus, the flange 108 provides a sealing engagement between the patient and the barrier to create a “floor” of the barrier that mitigates (or which can prevent) fluid flow from the interior volume and under the flange 108. In some non-limiting examples, the flange 108 can be pre-formed. For example, the flange 108 can be formed from reinforced rings, perimeters, etc., that are coupled to (or otherwise integrated within) the barrier 102. The reinforced rings, perimeters, etc., can be formed out of malleable materials (e.g., metals), so that personnel can customize the shape of the perimeter based on a particular procedure. In some cases, the reinforced rings, perimeters, etc., can be advantageous in that the flange 108 being pre-formed can be more quickly/easily installed to isolate a particular region of the patient.

In some non-limiting examples, the barrier 102 can have a single flange 108 or multiple flanges 108. For example, in the case of multiple flanges 108, the multiple flanges 108 can extend (or span) around particular regions of the perimeter of the barrier 102. This can be advantageous in that only specific locations may need flanges to provide an adequate seal that can adequately control the spread of pathogen harboring tissue particulates, or aerosols, thus minimizing installation time. In some non-limiting examples, the flange 108 can span along the entire portion of the perimeter of the barrier 102, and thus provide a higher degree of sealing (e.g., when engaged with the patient). In some non-limiting examples, a first flange 108 can extend into the interior volume, and a second flange 108 can extend away from the interior volume. In some cases, the first and second flanges 108 can be adjoining, while in other cases, the first and second flanges 108 can be separated along the perimeter of the barrier 102. By having two flanges, the surface area of the coupling layer that engages with a patient can be increased, which can provide a better seal. In some non-limiting examples, the flange 108 that extends into the interior volume can define the perimeter of the surgical opening.

In some non-limiting examples, the flange 108 can include a flap that is coupled to the barrier 102 and is situated between opposing ends of the barrier (e.g., along an axial direction of the barrier). This flap can extend around a portion or the entire perimeter of the 3D structure 104 of the barrier 102. Similarly to the other flanges, a lower surface of the flap can include a coupling layer (e.g., an adhesive). In this way, the flap can be pulled and coupled to the patient (or a structure), via the coupling layer, to help maintain the 3D structure 104 of the barrier 102. In other cases, the flap can be coupled to the patient or structure using a fastener (e.g., a clip, clamp, etc.).

In some non-limiting examples, the barrier 102 can have pleats, folds, and support layers 110 that can also help to maintain the 3D structure 104. The barrier 102 can have pleats, folds, support layers, or combinations thereof. The pleats can be implemented in many different ways. For example the pleats can be a knife pleat, a box pleat, a double box pleat, a cartridge pleat, etc. The pleats can be formed around particular regions of the barrier 102 in particular patterns to help maintain the desired 3D structure 104. For example, in the hemispherical or conical (or frustoconical) 3D structure 104, the pleats can be formed circumferentially around the barrier 102, whereas in the prisms 3D structure 104, the pleats can be formed on at or near the faces of the prims (e.g., but not on the edges of the prisms), or alternatively the pleats can be formed on at or near the edges of the prism (e.g., but not on the faces of the prisms). The folds (or creases) can also be formed in different ways. For example, the folds can be formed along an axial dimension of the 3D structure 104, and can span circumferentially (or span the perimeter) around the barrier 102 (e.g., in an accordion-like fashion). As another example, the folds can be formed along a circumferential direction (or along the perimeter) of the 3D structure 104, and can span the axial dimension (or a portion) of the 3D structure 104. The support layers can also be implemented in different ways. For example, the support layers can be formed out of strips of an adhesive (e.g., a polymer) coupled to the barrier 102 and which can alone provide rigidity and weight along specific portions of the barrier 102. For example, similarly to the pleats the support layers can be formed circumferentially, axially, along a perimeter, etc., of the barrier 102. In some non-limiting examples, the support layers can be situated within creases of the folds or pleats, which can further define the given crease, and which can maintain the structure of the crease (e.g., prevent the crease from being unfolded). In some cases, the support layer can be an adhesive that can couple together two portions of the barrier thereby forming a crease. In this case, the dimensions of the crease (e.g., how much the crease bends) can be determined based on the thickness of the support layer (or amount of material of the support layer).

In some non-limiting examples, the barrier 102 can include weights 112 (or a weight) that can also help to maintain the 3D structure 104. The weights 112 can be embodied in many different forms. For example, the weights 112 can be coupled to the interior surface of the barrier 102, coupled to the exterior surface of the barrier 102, integrated within the barrier 102 (e.g., encapsulated between two sheets), etc. The weights 112 can be formed out of various materials, such as, for example, metals, plastics, etc., and can be formed to embody different shapes and sizes. For example, the weights 112 can span a portion of the circumference (or perimeter) of the 3D structure 104. In some cases, the weights 112 can be situated near an axial end of the 3D structure 104 that is closer to the patient (e.g., than the opposing axial end of the 3D structure 104). This way, the weights 112 provide increased stability to an end of the barrier 102 that contacts the patient. In some non-limiting examples, the weights 112 can be positioned on the flanges 108 (or on the reinforcing member) to increase the bonding interface between the flange 108 and the subject. Similarly, the weights 112 can be positioned on tabs emanating from the barrier 102 (described in more detail below), that also can increase the bonding interface between the tab and the subject. In some cases, the weights can be formed out of a different material than the barrier 102. For example, density of each weight 112 can be greater than the density of the barrier 102. In addition, the mass of each weight 112 can be greater than the mass of the barrier 102. In some cases, a weight 112 can be in the form of a ring and is coupled to the barrier 102. In some cases, each of the weight(s) 112 can be rigid.

In some non-limiting examples, the barrier 102 also can include adhesive layers 114, which can also help to maintain the 3D structure 104. The adhesive layers 114 can span various portions of the barrier 102, and can have varying thicknesses. In some cases, the flanges 108 can include an adhesive layer 114 which can have a relatively large thickness to ensure proper sealing engagement with the patient. In some cases, the barrier 102 can include an aperture that defines the perimeter of the surgical opening, and can have an adhesive layer 114 that spans a surface of the barrier 102 that surrounds the aperture. This can ensure a proper securement of the aperture ensuring that the aperture does not move during the procedure. In some non-limiting examples, the barrier 102 is formed out of a contiguous piece, but which must be manipulated to form the 3D structure 104. For example, the barrier 102 can have a plurality of free ends emanating from a central region (e.g., containing the aperture). One or both of the free ends can include a strip extends from and along an edge of a given free end. The strip includes an adhesive layer 114 that can be secured to a surface of the opposing free end. This securement helps to define the 3D structure 104 and creates an adjoining edge between respective edges of respective free ends that mitigates fluid movement from the interior volume and into the exterior space along the adjoined edge. In some non-limiting examples, the barrier 102 can include tabs emanating from the exterior surface and having an adhesive layer 114 (or other coupling layer). The tabs can be situated near on or at the axial end of the 3D structure closer to the patient. The tabs can be coupled to the patient to provide additional securement locations along the patient, which can help to maintain the 3D structure.

In some non-limiting examples, the barrier 102 also can include a scaffold 116 that can also help to maintain the 3D structure 104. The scaffold 116 can generally act as a reinforcement system, a skeleton, etc., for the barrier 102 to provide a rigid support system for the barrier 102. Thus, the scaffold 116 can embody many different forms. For example, the scaffold 116 can include a plurality of support beams that can be coupled to the interior surface of the barrier 102, coupled to the exterior surface of the barrier 102, integrated within the barrier 102 (e.g., encapsulated between two sheets), etc. The support beams can be rigid, but can also be malleable so as to form different shapes for a particular procedure, such that the scaffold 116 can be customizable. The support beams can be formed out of metals, plastics, etc. The support beams can be oriented in many different ways or configurations. For example, the support beams can emanate out radially relative to an axial axis of the 3D structure 104, and can be curved along the axial axis. As another example, the support beams can be circumferentially oriented around the axial axis, and respective support beams can be separated along the axial axis. In some non-limiting examples, support beams (e.g., adjacent support beams) can be coupled together with other support beams. For example, two support beams that are circumferentially oriented around the axial axis can be coupled to a support beam that extends along the axial axis.

In some non-limiting examples, the scaffold 116 having the support beams can help to define the 3D structure 104 when the barrier 102 has a plurality of free ends emanating from a central region. For example, both of the free ends can include sleeves that are configured to (dimensioned, sized, etc.) receive a support beam. In this case, the support beam is received through both of the sleeves thus joining respective edges of the respective free ends. In some cases, one sleeve can be positioned above the other sleeve, such that the sleeves are coaxially aligned to receive a substantially straight support beam to join the respective edges of the respective free ends. In some cases, the sleeves both extend along the same axial dimension, and the support beam can have two axial portions, such that one axial portion is received through one of the sleeves, and the other axial portion is received through the other of the sleeves to join the respective edges of the respective free ends.

In some non-limiting examples, the scaffold 116, and in particular the support beams of the scaffold 116 can be formed out of a rigid material, or can be formed out of a flexible material. Regardless, the material of the scaffold 116 can be different than the material of the barrier 102. In some cases, the scaffold 116 can be formed out of a metal, a plastic, etc.

In some non-limiting examples, the barrier 102 also can include instrument attachments 118 that can also help to maintain the 3D structure 104. The instrument attachments 118 can be coupled to an interior surface of the barrier 102, an exterior surface of the barrier 102, or integrated within the barrier 102, and generally allow the barrier 102 to be secured to the particular instrument to provide a securement location that can stabilize the barrier 102 and to maintain the 3D structure 104. As an example, the instrument attachment 118 can include an elastic ring (or other shape) that can expand and retract over a portion of the instrument to generate a sealing engagement with the portion of the instrument. As another example, the instrument attachment 118 can include a cuff (e.g., of a shape corresponding to the instrument) that can be removably coupled (e.g., hook and loop fasteners, magnetically coupled, etc.) to itself to adjust the size of the perimeter of the cuff. Thus, the specific instrument can be inserted into the cuff and a free end of the cuff can be used to tighten the engagement (e.g., decrease the perimeter) and can then be coupled to a location of the cuff to securely fasten the instrument to the instrument attachment. As yet another example, the instrument attachment can be an elastic (or otherwise membranous) sheet having a hole therethrough. The particular instrument can be inserted into the hole and the elastic material retracts around the instrument to provide a sealing engagement with the portion of the instrument. In some cases, a region of material weakness (e.g., a perforation) can be located below the instrument attachment 118 so as to allow quick removal and containment of potential airborne particulates or aerosols within the interior volume when the barrier 102 is deconstructed (e.g., upon completion of the procedure). In some cases, the instrument is a magnifying instrument (e.g., a microscope, an endoscope, an exoscope, etc., such as for use in an otolaryngology procedure). In some cases, the instrument attachment 118 can be a hole with an adhesive layer that extends partially or entirely around the hole. In this way, the adhesive layer can be coupled to a portion of the instrument 118. In other cases, the instrument attachment 118 can include an adjustable tightening component (e.g., an elastic band, a cable tie, an irised mechanism, etc.). In some configurations, an adhesive layer can be positioned on the interior surface of the barrier 102, which can partially (or fully) extend around (e.g., circumferentially) the barrier 102.

In some non-limiting examples, the instrument attachment 118 can be for use with an operating room light. For example, the instrument attachment 118 can include two straps coupled to the barrier 102 that can be received around respective ends of the operating room light. In some cases, this can include the handles of the operating room lights. In some non-limiting examples, the straps can be elastic to retractably engage the operating room light (or a component thereof, such as the handles of the operating room light). The specific engagement between the instrument attachment 118 with the operating room light can be advantageous for a number of reasons. First, this is a component that is typically always present in the operating room. Second, the operating light can provide ample light to the interior volume of the 3D structure, which can prevent the need for other light sources to visualize the contents situated in the interior volume. In some non-limiting examples, the instrument attachment 118 can generally be any supporting object in an operating room (e.g., a bed, the operating room light described above, etc.).

In some non-limiting examples, the isolation system 100 can include arm ports or sleeves 120. In some non-limiting examples the arm port(s) 120 are simply apertures directed into the barrier 102 and configured (e.g., dimensioned, sized, etc.) to receive an arm of personnel conducting the procedure. In some non-limiting examples, the arm port 120 can include a flap that is removably coupled to the exterior surface of the barrier 102 (e.g., with hook and loop fasteners, magnetically coupled, etc.) and that covers (or spans across) the entire (or a portion of the) aperture to provide a seal (e.g., when not in use). In some non-limiting examples, the aperture, and more specifically the edges of the barrier 102 that define the aperture can interface with (e.g., be coupled to) a rigid attachment (e.g., having a rigidity greater than the material of the barrier 102) that can more reliably define the arm port 120. For example, the rigid attachment can have a peripheral slot that receives the edges of the barrier 102 that define the aperture, and can have a hole therethrough that can be configured to receive an arm of personnel. In some non-limiting examples, the arm port 120 can include a valve that generates a seal when the valve is not in use, and when an arm of one of the personnel is received into the valve. Thus, the valve can be integrated with the aperture, with the rigid attachment, etc. In some non-limiting examples, the valve can have a plurality of flaps that retract to generate a seal. In some non-limiting examples, the arm port can include an elastic cuff (or otherwise membranous sheet having a hole therethrough) that can provide a sealing engagement when an arm is inserted. In other words, the elastic cuff or membrane expands to retract around the arm. In some cases, the elastic cuff (or the membranous sheet) are situated entirely or partially around the arm port 120. In some non-limiting examples, the arm port 120 can include a rigid band (e.g., a ring) that can define the aperture of the arm port 120. For example, the rigid band can be coupled to or integrally formed with the barrier 102, which can make insertion and removal of an arm through the arm port 120 easier. In some cases, the arm port 120 can include a cable tie (or in other words a zip-tie). In this way, the cable tie can be coupled to the barrier 102 and can surround the aperture of the arm port 120 to be adjusted. For example, the aperture of the arm port 120 can be decreased (e.g., around the arm of the personnel) by pulling the cable tie. In some configurations, a region of material weakness of the barrier 102 can be situated between the arm port 120 (e.g., configured as a hole) and the barrier 102. For example, the region of material weakness (e.g., a perforation) can extend partially (or entirely) around the arm port 120. In this way, the arm port 120 can be decoupled from the barrier 102 (e.g., after the surgical procedure has been completed).

In some non-limiting examples, the arm ports 120 can include a plug that can be received in the arm port 120 to prevent fluid from flowing from the interior volume and into the exterior space. The plug can embody many different forms. For example, the plug can be rigid and can be inserted into the aperture so that the material of the barrier 102 or the elastic cuff (or membranous sheet) expands and contracts around the plug to provide a sealing engagement (e.g., when the arm port 120 is not in use). In some non-limiting examples, such as with the rigid attachment, the plug can be elastic to expand around the rigid attachment to provide a sealing engagement. In other non-limiting examples, such as with the rigid attachment the plug (e.g., rigid) can be removably coupled the rigid attachment (e.g., magnetically coupled, threadingly engaged, hook and loop fastened, etc.).

In some non-limiting examples, the isolation system 100 can include arm sleeve(s) 120. In some non-limiting examples, the arm sleeves 120 are integrally formed with and extend outwardly away from the barrier 102. Thus, the integrally formed arm sleeves 120 can be inverted and inserted into the interior volume of the 3D structure 104 to prevent direct contact between the arm (or hand) of personnel and the patient. In some non-limiting examples, such as with the rigid attachment, the isolation system 100 can include an arm sleeve 120 having a rigid cuff that can be removably coupled to the arm port. For example, the rigid cuff of the arm sleeve 120 can be threadingly engaged with the arm port. Then, the arm sleeve 120 can be inverted into the interior volume of the 3D structure to prevent direct contact between the arm of personnel and the patient. In some non-limiting examples, the arm sleeve 120 can have an area of material weakness (e.g., a perforation) that, for example, can allow a hand portion of the arm sleeve 120 to be removed if personnel need better gripping. Additionally or alternatively, the arm sleeve 120 can also include a first portion that is removably coupled to a second portion. Again, this can allow personnel to easily remove the first portion from the second portion of the arm sleeve 120 if personnel need better gripping. In some cases, an area of material weakness (e.g., a perforation) can extend (partially or entirely) around a portion of the barrier 102 (e.g., the sleeve 120). In this way, the arm port 120 can be created when desired (e.g., just before conducting the procedure) so that when not in use fluid flow (and thus contaminants) are mitigated from flowing through the barrier 102 (e.g., because the arm port 120 has not been opened).

In some non-limiting examples, each arm sleeve 120 can include a glove positioned at the end of the sleeve 120. In some cases, the glove can be coupled to (or integrally formed with) the arm sleeve 120. In some cases, the glove can be formed out of the same material as the sleeve 120, or can be formed out of a different material (e.g., a material that has better gripping properties, such as a rubberized material, a textured material, etc.). In some configurations, each arm port 120 can have a sleeve 120 emanating from the barrier 102 (e.g., so the arm port 120 is coaxially positioned relative to sleeve 120, so the sleeve 120 surrounds he arm port 120). In some cases, the sleeve 120 can extend into the interior volume of the barrier 102. In addition, a free end of each sleeve 120 can be truncated (e.g., with the free end, or glove removed) so that when an arm of the personnel is positioned through the arm port 120 and the sleeve 120, the hand of the personnel extends past the sleeve 120 (e.g., so that the surgeon can manipulate instruments easier). In some cases, the length of the sleeve 120 can be varied to accommodate different arm sizes, or so that different amounts of an arm of a person extend past a free end of the sleeve 120.

In some non-limiting examples, the arm ports 120, the arm sleeves 120, or both can be appropriately positioned relative to other structures of the isolation system 100. For example, the arm ports 120, the arm sleeves 120, or both can be positioned below the scaffold 116. This way, the arms of personnel have enough clearance away from the scaffold 116 that may otherwise undesirably contact a portion of the arm sleeve 120 (e.g., potentially puncture a sleeve, or impede movement of the personnel arms). As another example, the arm ports 120, the arm sleeves 120, or both can be positioned below, above, or between the weights 112. In some non-limiting examples, with the weights 112 below the arm ports (or sleeves) 120, the arm ports (or sleeves) 120 can be less prone to movement (e.g., as the weights 112 mitigate movement of the barrier 102). In some non-limiting examples, with the weights 112 above the arm ports (or sleeves) 120, the weights 112 can provide additional sealing properties for the arm ports (or sleeves) 120.

Regardless of the structure of the arm ports (or sleeves) 120, each of the arm ports (or sleeves) generally provide a passage from the exterior space and into the interior volume of the 3D structure 104 so that a practitioner (and more specifically the arm of personnel) can interact with the patient within the interior volume of the structure 104. In some non-limiting examples, a given isolation system can include two arm ports or sleeves on each side of the barrier. For example, a generally cylindrical isolation shape (or other shape) can include eight arm ports or sleeves (e.g., so that other personnel can interact with the interior volume of the 3D structure).

In some non-limiting examples, each of the arm ports 120 (and corresponding sleeves 120) can be closed when not in use. For example, the arm ports 120 can be plugged, fastened, etc., to reduce the size of the aperture of the corresponding arm port 120. In addition, the sleeves 120 can be compressed (e.g., rolled up) for later expansion during use. For example, a fastener (e.g., a clasp, a clip, etc.) can extend around the compressed arm sleeve 120.

In some non-limiting examples, the isolation system 100 can include instrument port(s) 122. The instrument ports 122 can be similarly structured as the previously described arm ports (or sleeves) 120, and the instrument attachments 118, and thus these similar structures also pertain to the discussion of the instrument ports 122. The instrument ports 122 can be configured to (e.g., dimensioned, sized, etc.) receive various instruments for manipulating structures of the patient, such as, for example, surgical instruments that have cords. In some non-limiting examples, the instruments ports 122 can have sleeves attached, which can guide the cords of the instruments. In some configurations, the instrument ports 122 can be smaller than the arm ports 120.

In some non-limiting examples, the isolation system 100 can have a sanitation system 124, which will be described in more detail below. Generally, the sanitation system 124 provides components, features, structures, etc., that aid in the removal, identification, entrapment, or inactivation of pathogen laden aerosols and tissue particulates. For example, the sanitation system 124 can include suction ports, suction attachments, suction systems (e.g., sources), suction bags, coatings, charged properties (e.g., of the barrier 102), filters, debris detections systems having light sources and image sensors, other fluid ports (including those that connect to a pump to drive a fluid, such as air, into the interior volume of the barrier), pumps (e.g., air pumps), etc.

In some non-limiting examples, the isolation system 100 can include a disposal system 126. The disposal system 126 can be embodied in many different forms, and generally the disposal system 126 encloses off the interior volume of the 3D structure 104 for disposal of the isolation system 100 (e.g., upon completion of the procedure). For example, the disposal system 126 can be a loop of material that can be tied off in many different ways. As another example, a retaining ring (e.g., situated on a flange) can be crumpled to seal off the interior volume of the 3D structure 104 (e.g., after removal from the patient). As yet another example, the disposal system 126 can include clamps that close off respective ends of the 3D structure 104. In some cases, the disposal system 126 can include zippers that can be decoupled to retain the potentially pathogen laden air within the internal volume of the 3D structure prior to disposal of the barrier. In some configurations, opposing ends of the barrier 102 can each have a closing device. For example, one end of the barrier 102 (e.g., disposed under the instrument attachment 118) that is farthest away from the patient (or sample) can include a first closing device, and an opposing end of the barrier 102 that is closest to the patient can include a second closing device. Each of the closing devices separate two volumes at the closing device, and with two closing devices deployed (e.g., closed off), the interior volume of the barrier 102 can be isolated from the ambient environment. In this way, the aerosol and other particulates can be trapped after the procedure is complete. The closure devices can be a tie, a cable tie, an adhesive layer (e.g., that circumferentially extends around a portion or all of the barrier 102), etc. In some cases, an area of material weakness (e.g., a perforation) of the barrier 102 can be situated above the first closure device, and below the second closure device. In this way, with both closure devices deployed, the trapped interior volume of the barrier 102 with the closure devices deployed can be easily separated from the other portions of the barrier 102 at the two areas of material weakness.

In some non-limiting examples, the isolation system 100 can include a viewing window 129. The viewing window 128 can be integrated within the barrier 102, or can span across a hole in the barrier 102. The viewing window 128 generally allows for a practitioner to visually see through the viewing window 128 to help to facilitate the procedure. So, the viewing window 128 can be formed out of a material that allows light of at least one wavelength within the visible spectrum (e.g., light having a wavelength between 380 nm and 790 nm) to pass through. The viewing window 128 can be located at various locations on the barrier 102. For example, the viewing window 128 can be located on an axial end of the 3D structure 104 that is farther away from the patient (e.g., the opposing axial end of the 3D structure 104 being closer to the patient). The viewing window 128 can embody many different sizes, and shapes. For example, the viewing window 128 can be circular, square, rectangular, etc. In some non-limiting examples, the viewing window 128 can be partially (or fully) transparent to allow the personnel to view the contents within the interior volume of the 3D structure. In some cases, the viewing window 128 can be formed out of a rigid material (e.g., a clear plastic material). In some configurations, the viewing window 128 (e.g., the interior surface) can be electrically charged to repel particles from being adsorbed onto the surface of the window 128. In this way, the view of the viewing window 128 is not obscured.

In some non-limiting examples, isolation system 100 can include a pocket (or multiple pockets), and a wipe situated within each pocket (e.g., each pocket configured to receive a wipe). The pocket (and the corresponding wipe) can be situated within the interior volume of the barrier 102. In this way, if condensation forms (or particulates adsorb) on the viewing window 128, a practitioner can retrieve the wipe from the pocket, remove the wipe, and wipe off the condensation (or particulates) to improve viewing through the viewing window 128. In some cases, the wipe can be a piece of cloth, fabric, etc. The wipe can be soaked in a compound (e.g., a cleaning compound to facilitate cleaning of the viewing window 128).

In some non-limiting examples, the isolation system 100 can include an additional barrier (e.g., similar to the barrier 102) that is coaxially positioned relative to the barrier 102. For example, this additional barrier can be positioned so that the barrier 102 surrounds the additional barrier. In some cases, the additional barrier can include any of the components of the isolation system 100 (or any isolation system described). For example, the additional barrier can include an instrument attachment, a flange, etc. In this way, the additional barrier can further mitigate undesirable fluid flow from the interior volume and into the exterior space.

Although the above description of the isolation system 100 was in reference to personnel conducting an operatory procedure on a patient (e.g., a surgery on a patient such as, a neurosurgical procedure, a general surgery procedure, a vascular surgery procedure, an ear nose and throat (“ENT”) surgery procedure, an internal medicine procedure, an anesthesia procedure, a gastrointestinal procedure, a genitourinary procedure, an intensive care unit (“ICU”) procedure, an emergency medicine procedure, an orthopedic procedure, other aerosol generating procedures, etc.), it can be appreciated that in alternative non-limiting examples the isolation system 100 can be utilized for laboratory procedures, experiments, tests, etc., where a practitioner may interact with a sample that can spontaneously generate pathogen laden aerosols, tissue particulates, etc., or which can generate pathogen laden aerosols, tissue particulates, etc., during manipulation of the sample (e.g., during drilling of the sample, etc.). For example, these samples can include tissue biopsies, pathology screenings, testing swabs (e.g., from the SARS-CoV-19), etc.

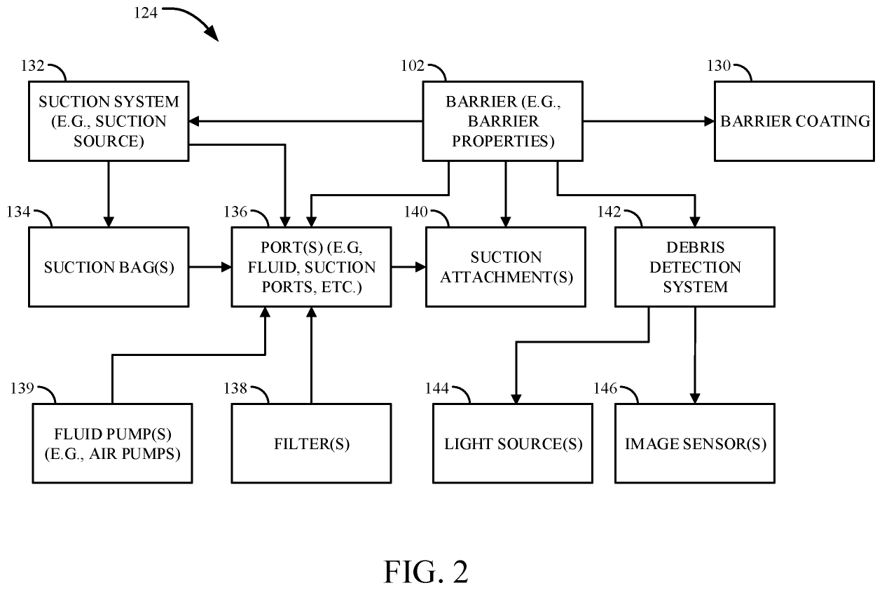

FIG. 2 shows a schematic illustration of an example of the sanitation system 124. The sanitation system 124 can include the barrier 102, a barrier coating 130, a suction system 132, suction bags 134, ports 136, filters 138, suction attachments 140, and a debris detection system 142. The barrier 102 can have the barrier properties 106 that can deter or can capture pathogen laden aerosols or tissue particulates. For example, the interior surface of the barrier 102 can be formed of (or treated with) a material that is charged (e.g., positively charged), which can either attract opposing charged aerosols or tissue particulates, or repel similarly charged aerosols or tissue particulates. In some non-limiting examples, the interior surface of the barrier 102 can have some portions that have a charge, and some portions that have an opposing charge. For example, assuming that some types of particles have a known charge type, portions of the interior surface of the barrier 102 far away from a suction port can have a charge known to repel the particles, whereas portions of the interior surface closer to the suction port can have a charge known to attract the particles. This can, for example, force tissue particulates and aerosols to be positioned along an axial axis of the 3D structure 104 (e.g., centrally) away from the suction port, and to be attracted towards the suction port to be readily evacuated (e.g., assuming that the suction port is aligned with the axial axis of the 3D structure 104). In some non-limiting examples, the barrier 102 can be charged to repel the tissue particulates and aerosols into the flow path of the suction system 132.

In some non-limiting examples, the entire (or a portion of) the interior surface of the barrier 102 can be coated with (or formed out of) a materials that entraps or inactivates pathogens, tissue particulates, and aerosols. For example, the interior surface of the barrier 102 can include an adhesive layer that can entrap tissue particulates. In some cases, the interior surface of the barrier 102 can include chemicals, drugs, a medicant, etc., that can deactivate (or otherwise “kill”) pathogens. For example, the entire (or a portion of) interior surface of the barrier 102 can be treated with an anti-infectious agent, such as antibacterial compounds, anti-viral compounds, anti-fungal compounds, a layer of (liquid) bleach (e.g., sodium hypochlorite), activated carbon, a peroxide (e.g., hydrogen peroxide), etc. In some cases, the entire (or a portion of) the interior surface of the barrier 102 can be coated with (or formed out of) a material that attracts the aerosols (or particulates), such as having an electric charge, having a particular phobicity (e.g., hydrophobic), etc.

In some non-limiting examples, the barrier coating 130, which can be applied to the exterior or interior surface of the barrier 102 can be an anti-reflective coating, where the barrier 102 allows at least some visible light through. In other cases, the barrier 102 itself can be formed of a material that allows at least some visible light through and is anti-reflective. In other cases, the isolation system can include lights (e.g., incandescent, light emitting diodes, etc.) that can either be positioned in the exterior space, or the interior volume, or both, to allow better visualization of the contents within the interior volume (e.g., especially with the anti-reflective coating(s)).

In some non-limiting examples, the sanitation system 124 can include a suction system 132. In some non-limiting examples, the suction system can be a medical aspirator, a suction machine, etc. Thus, the suction system 132 can include a suction probe that supplies a suction source, a motor, tubing, filters, etc. In some non-limiting examples, the suction system 132 is part of a high-efficiency particulate air (“HEPA”) filtration system that can include tubing that supplies the suction source (to the HEPA filter).

In some non-limiting examples, the sanitation system 124 can also include suction bags (or a suction bag) 134. In some non-limiting examples, the suction bags 134 are coupled to and are in fluid communication with the interior volume of the 3D structure 104. Each suction bag 134 can include a hole directed therethrough that can receive a component of the suction system 132 (e.g., a suction probe, tubing leading to a suction system, etc.). In some non-limiting examples, the suction bag 134 is integrally formed with the barrier 102. In other non-limiting examples, the suction bag 134 can be coupled to a rigid cuff that can interface with a port (e.g., the instrument ports, the arm ports, the suction ports, etc.). In some cases, the suction bag 134 has a cross-sectional area that decreases from the coupling location to the barrier 102 and until it reaches the hole of the suction bag 134. In some non-limiting examples, the suction bags 134 can include ports (e.g., fluid ports), coatings (e.g., barrier coatings), charged properties, etc. In some non-limiting examples, the suction bags 134 can be positioned relative to various structures of the isolation system 100. For example, a suction bag 134 can be positioned on the same side of the barrier 104 as the arm ports (or sleeves) 120. This can be advantageous at least because the fluid flow path defined by the suction source can be relatively close to the arms of personnel (e.g., that conducts the procedure). In some cases, the suction bag 134 can be positioned entirely (or partially) within the interior volume of the 3D structure 104 of the barrier 102.

In some non-limiting examples, the sanitation system 124 can include port(s) 136. The ports 136 can be similarly structured as the previously described arm ports (or sleeves) 120, the instrument attachments 118, and the instrument ports 122, and thus these similar structures also pertain to the ports 136. For example, the ports 136 can simply be apertures (of various sizes and shapes) directed into the barrier 102, the ports 136 can have rigid attachments with holes directed therethrough, or the ports 136 can be (or include) valves as previously described. In some non-limiting examples, the ports 136 can be fluid ports that allow fluid to flow from the exterior space and into the 3D structure 104 of the barrier 102. In other non-limiting examples, the ports can be suction ports that are configured to receive tubing, a probe, etc., from a suction source (e.g., a component of the suction system 132). In some configurations, a port 136 can be a one way valve that is configured to allow fluid flow from the exterior space and into the interior volume (e.g., of the 3D structure 104 of the barrier 102), but which prevents fluid flow from the interior volume and to the exterior space. In this way, the as fluid is evacuated by the suction source, fluid (e.g., air) is replenished within the interior volume of the 3D structure 104 of the barrier 102 to maintain a substantially (e.g., deviating by less than 20%) constant interior volume.

In some non-limiting examples, the sanitation system 124 can include filters 138. The filters 138 can have varying thicknesses, shapes, etc., can be coupled to the barrier 102, and can be inserted into a flow path (e.g., into the suction bag 134). In some non-limiting examples, the filters 138 can be in sealing engagement with a port 136, which can be accomplished by coupling the filter 138 to the boundary of the port, or coupling the filter 138 to the barrier 102 so that the filter 138 spans across the entire port 136. In some non-limiting examples, the filters 138 can allow a certain percentage of airborne particles to pass through (e.g., 95%, being an N95 filter), and can have pore sizes that only allow certain sized particles to pass through.

In some non-limiting examples, the sanitation system can include fluid pumps 139, which are configured to drive fluid into the interior volume of the 3D structure. This way, “clean” fluid (e.g., air) can be introduced into the interior volume of the 3D structure, can interact with the fluid within the interior volume, to be evacuated by the suction system. A conduit, attachment, etc., of the fluid pump 139 can be interfaced with one of the fluid ports 136, and can be in sealing engagement with the respective fluid port 136.

In some non-limiting examples, the sanitation system 124 can include suction attachments 140. The suction attachments 140 can be fitted attachments that allow a probe, tubing, etc., of a suction source to be temporarily coupled to the suction attachment 140. For example, the suction attachment 140 could be a female attachment that allows the component of the suction source to be temporarily snap-fitted (e.g., interfacing with a male attachment). As another example, the suction attachment 140 could be threaded to threadingly engage the component of the suction source. As yet another example, the suction attachment 140 could be spring clip that when depressed allows jaws to retractingly engage the component of the suction source (or when depressed releases the engagement of the spring clip). In some non-limiting examples, and generally, the suction attachments 140 can emanate from a port 136.

In some non-limiting examples, the sanitation system 124 can include the debris detection system(s) 142. The debris detection system 142 can be in communication with the suction system 132, such that when the debris detection system 142 determines that a density of particles of the internal volume of the 3D structure 104 exceeds a threshold value, the debris detection system 142 can turn on the suction system 132, or can increase the output of the suction system 132 (e.g., increase the volume per unit of time). As shown the debris detection system 142 includes light sources 144, and image sensors 146, although it can be appreciated that other optical components could be utilized (e.g., lenses, prisms, etc.). In some cases, the light source 144 and the image sensor 146 can be coupled to the interior surface of the barrier 102. The light sources 144 can sufficiently illuminate the interior volume of the 3D structure 104. In some cases, some light wavelengths not emitted by the light sources 144 reflects off of a surface of the barrier 102. So, in this case, the interior surface (or exterior surface) of the barrier 102 can have a reflective coating (e.g., an ultraviolet or infrared reflective coating). In some cases, the light sources 144 are ultraviolet light sources, while in other cases, the light sources 144 are infrared light sources. In some configurations, the light source 144 can be a (low powered) UV light source (e.g., a UV LED) which can be coupled to the interior surface of the barrier 102. The UV light source can thus highlight areas of particulate (or aerosol) dispersion. In some cases, the debris detection system 142 can be a spectrometer (e.g., an optical spectrometer).

The image sensor(s) 146 can be embodied in different forms, such as a charged coupled device (“CCD”), active pixel sensors (e.g., CMOS image sensors). The image sensors 146 can be selective for the light produced by the light sources 144. This way, background light that may negatively impact image data acquired from the image sensors 146 can be mitigated. The debris detection system 142, although not shown, can include processors, displays, memory, and other typically used computing components. In some non-limiting examples, the debris detection system 142 can be located within the interior volume of the 3D structure 104, or can be positioned in the exterior space but in optical communication with the interior volume of the 3D structure 104 (e.g., an aperture of the debris detection system 142 can be aligned with an aperture of the 3D structure 104). In some non-limiting examples, the components of the debris detection system 142 can be packaged together within a housing. In some cases, the debris detection system 142 can include the barrier coating 130. For example, a color-changing compound can be disposed over the entire (or portions of the) interior surface of the barrier 102, which when contacted with debris, changes from one color to a different color. In some cases, rather than a coating the barrier 102 can be formed with the color-changing compound.

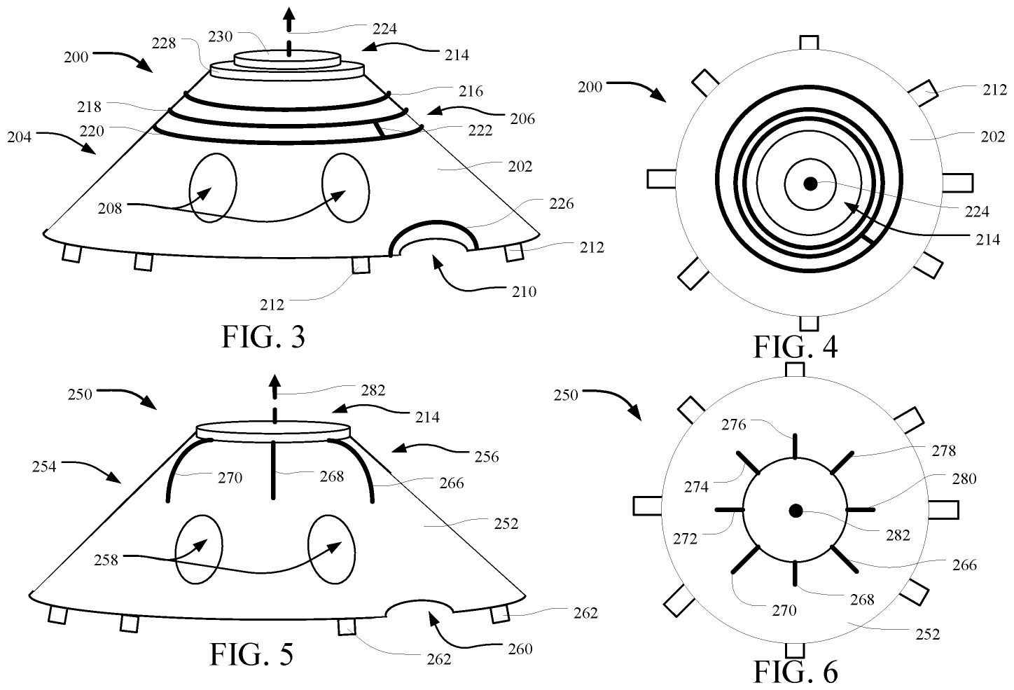

FIGS. 3 and 4 show an example of an isolation system 200. The isolation system 200 pertains to the previously described isolation systems and thus features of the other previously described isolation systems can be implemented with this isolation system and vice versa. The isolation system 200 can also include a barrier 202 having a 3D structure 204, a scaffold 206, arm ports 208, another arm port 210, tabs (or a tab) 212, and a microscope attachment 214. As shown, the scaffold 206 is shown being coupled to the interior surface of a substantially translucent barrier 202. The scaffold 206 can include support beams 216, 218, 220, which are coaxially displaced from each other along the axial axis 224 of the 3D structure 204, and are curved about the axial axis 224. The support beams 216, 218, 220 are positioned above the arm ports 208, which can prevent unwanted interaction between personnel's arm and the scaffold 206. The scaffold 206 can include a support beam 222 that is coupled to and between the support beams 218, 220, and that extends along the axial axis 224 of the 3D structure 204. Although only a single support beam 222 is shown extending substantially along the axial axis 224, in alternative configurations other support beams similar to the support beam 222 can be included. The different orientations of the support beams of the scaffold 206 can provide increased structural support for the 3D structure 204. Additionally, the coaxially positioning of the support beams can allow for relatively easy installation or deconstruction of the 3D structure 204. For example, opposing ends of the barrier 202 can be grasped and pulled to install, or pushed to deconstruct the barrier 202. In some cases, the deconstructed state of the isolation system 200 being substantially flat can be advantageous for packaging, transport, etc. In some non-limiting examples, the support beams can be formed of malleable materials such as metals so that personnel can tailor the 3D structured 204 to his or her desired shape.

In some non-limiting examples, a support beam 226 can help to define the arm port 210. For example, the support beam 226 is coupled to the interior surface and near a free end of the barrier 102. The support beam 226 can be curved as shown and can define a maximum size of the arm port 210. For example, when an arm of a practitioner enters the arm port 210, the barrier 202 can be compressed (e.g., folded) until it reaches the boundary defined by the support beam 226. Thus, the support beam 226 can limit the size of the arm port 210. As shown, the isolation system 200 can include a plurality of tabs 212 that are coupled to (or are integrally formed with) the exterior surface of the barrier 202. The tabs 212 can have a coupling layer that couples (or removably couples) the respective tab 212 to a patient, a structure (e.g., a patient bed, a operatory table), etc. In some cases, the coupling layer is a hook or a loop faster, includes a magnet, etc. In other cases, the coupling layer is an adhesive. In some configurations, although the tabs 212 are illustrated as being situated at an edge of the barrier 202, the tabs 212 can be situated between opposing ends of the barrier 202 along the axial axis 224. In this way, a free end of the barrier 202 can be folded under itself (e.g., an upper portion of the barrier 202), while the tabs 212 can decrease relative movement between the isolation system 200 and the structure (or person) that the tabs 212 are coupled thereto.

The microscope attachment 214 can be embodied in many different forms as described above. However in the illustrated non-limiting example of FIGS. 3 and 4, the microscope attachment 214 can include a rigid attachment 228 that can be removably coupled to a typical microscope (e.g., those used in otolaryngology procedures), and that is coupled to the barrier 102. The rigid attachment 228 can have threads (not shown) that can threadingly engage opposing threads of the microscope. The microscope attachment also can include a lens 230 that is coupled to the rigid attachment 228. Although not shown, the rigid attachment 228 has a hole that allows light to pass through the lens, into the hole of the rigid attachment 228, and into the interior volume of the 3D structure 104 (or vice versa). The microscope attachment 214 having a lens can be advantageous at least because in some cases, the lenses of typical microscopes are disposed after a procedure. In some non-limiting examples, the isolation system 200 can include a regular microscope drape (not shown). In this case, the regular microscope drape can be positioned exteriorly or interiorly relative to the barrier 202.

FIGS. 5 and 6 show another example of an isolation system 250, demonstrating a different implementation of a scaffold. The isolation system 250 pertains to the previously described isolation systems and thus features of the other previously described isolation systems can be implemented with this isolation system and vice versa. For example, the isolation system 250 can include similar components to that of the isolation system 200. As shown, the isolation system 250 can include a barrier 252 having a 3D structure 254, a scaffold 256, arm ports 258, another arm port 260, tabs 262, and an instrument attachment 264. The scaffold 256 can include support beams 266, 268, 270, 272, 274, 276, 278, 280 that extend along, and which extend outward from the axial axis 282 of the 3D structure 254 in opposing radial directions. The support beams 266, 268, 270, 272, 274, 276, 278, 280 also extend along the axial axis 282, and are curved. The support beams 266, 268, 270, 272, 274, 276, 278, 280 are shown being equidistant from each other, although in other configurations, other separation distances are possible.

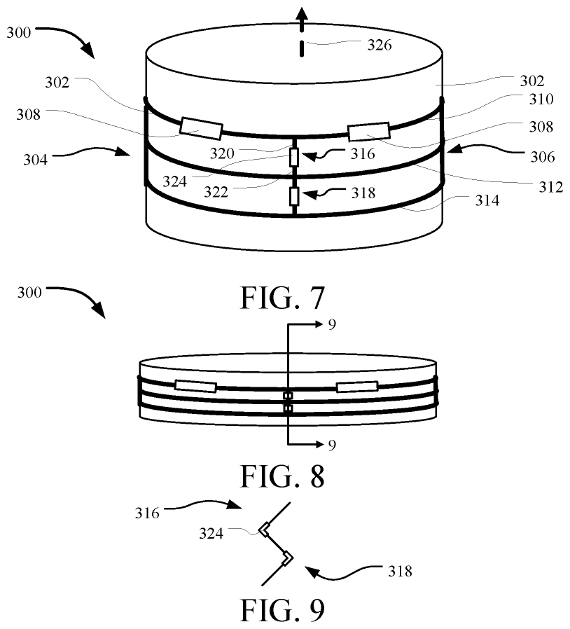

FIGS. 7-9 show another example of an isolation system 300, demonstrating another different implementation of the scaffold. The isolation system 300 pertains to the previously described isolation systems and thus features of the other previously described isolation systems can be implemented with this isolation system and vice versa. For example, the isolation system 300 can include similar components to that of the isolation systems 200, 250. As shown, the isolation system 300 can include a barrier 302 having a 3D structure 304, a scaffold 306, and sleeves 308. The scaffold 306 which is situated exteriorly relative to the 3D structure 304 can include support beams 310, 312, 314 that are circular, and that are axially displaced from each other along the axial axis 326 of the 3D structure 304. The support beam 310 is securely received though the sleeves 308 to couple the support member 310 to the barrier 302. Although two sleeves 308 are illustrated in FIG. 7, other numbers and orientations of sleeves can be used to contain the support members. Additionally, the sleeves could be coupled to the interior surface of the barrier 302. In some cases, the scaffold 306 can be coupled to the barrier 302.

As shown, the scaffold 306 also can include support beams 316, 318, which generally extend along the axial axis 326. The support beam 316, which is similar to the support beam 318, has (substantially) rigid members 320, 322 joined by a resilient member 324. The rigid members 320, 322 can be formed of various materials (e.g., plastics), and the resilient member 324 can also be formed of various materials (e.g., silicone, rubber, etc.). Additionally, the rigid members can be shared between support members, such as the rigid member 322 being shared between support beams 316, 318. The resilient member 324 allows the rigid members 320, 322 to be displaced closer or farther away from each other. For example, FIG. 8 shows the isolation system 200 in a compressed state (e.g., when packaged, or when desired to be deconstructed), and FIG. 9 shows a cross-sectional view of the support beams 316, 316 in the compressed state. As shown in FIG. 9, the resilient member 324 bends to allow the rigid members 320, 322 to be displaced closer to each other. These support beams 316, 318 can be advantageous at least because the rigid members can provide structural integrity for the 3D structure 304, and the resilient portions allow the rigid portions to be moved accordingly.

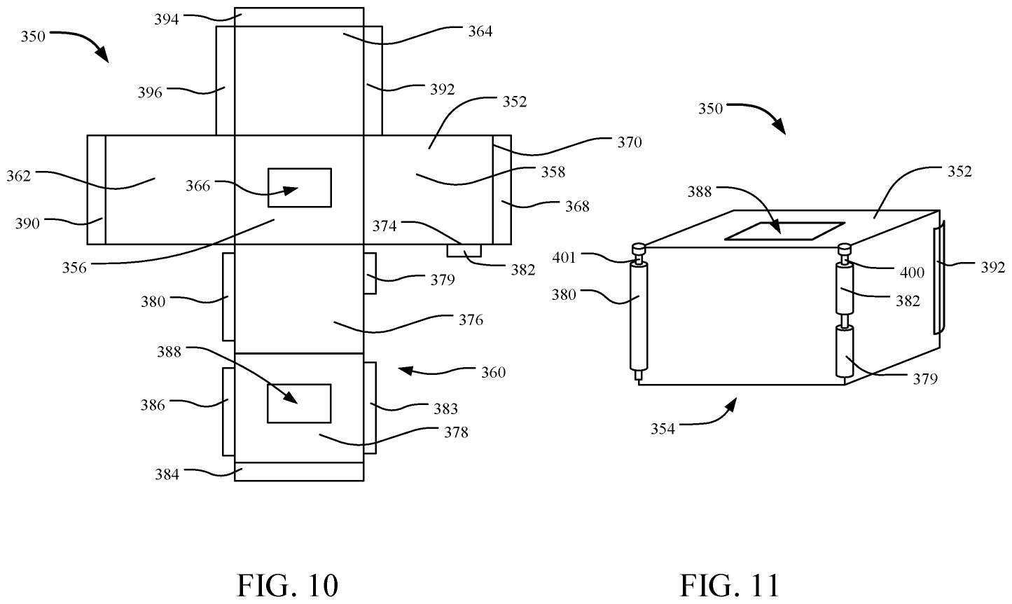

FIG. 10 shows another example of an isolation system 350, demonstrating a different implementation of a 3D structure. The isolation system 350 pertains to the previously described isolation systems and thus features of the other previously described isolation systems can be implemented with this isolation system and vice versa. For example, the isolation system 350 can include similar components to that of the isolation system 200, 250, 300. The isolation system 350 can include a barrier 352 that forms into a 3D structure 354, a central region 356, and free ends 358, 360, 362, 364 emanating from the central region 356. As shown in FIG. 10, the barrier 352 (and more specifically the 2D structure) is illustrated as being a single contiguous piece that can be formed into the 3D structure 354 of FIG. 11. Although the 2D structure of the barrier 352 in FIG. 10 forms the 3D structure 354 of FIG. 11 where the 3D structure 354 is illustrated as being a rectangular prism, in other configurations, other shapes can be formed, such as cones, rectangular prisms, etc.

The central region 356 has a first surface and an opposite second surface, and surgical opening 366 therethrough. The surgical opening 366 is illustrated as being a rectangle, although other shapes, and sizes of the surgical opening 366 are contemplated. The first surface of the central region 356 faces out of the page relative to the view of FIG. 10, and the opposite second surface of the central region 356 faces into the page relative to the view of FIG. 10. The second surface of the central region 356 can include an adhesive layer (or coupling layer) that spans the entire (or a portion of the entire) second surface. In some cases, the adhesive layer can include a backing (e.g., a plastic backing) that can be removed prior to securement of the adhesive layer to the patient.

As shown, the free end 358 can include a strip 368 that is coupled to an extends along an edge 370 of the free end 358 that has a coupling layer. In some cases, the strip 368 is the barrier 352, while in other cases, the strip is a different material than the barrier 352 that can be coupled to the edge 370. Additionally, in the case of the strip 368 not being a portion of the barrier 352, the strip 368 can be coupled to a different portion of the barrier 352 (e.g., one of the surfaces of the barrier 352). The free end 358 also can include a sleeve 372 that is coupled to and spans along a portion of the edge 374 of the free end 358. Similarly to the strip 368, the sleeve 372 can be coupled to different portions of the barrier 352.

In some non-limiting examples, the free end 360 has a first region 376 coupled to a second region 378. The free end 360 also can include sleeves 379, 380 that are coupled to respective edges of the free end 360, although other coupling locations are contemplated. The second region 378, which is situated farther away from the central region 356 than the first region 376 also can include strips 383, 384, 386 that are coupled to respective edges of the second region 378, although other coupling locations are contemplated. Each of the strips 383, 384, 386 also include a coupling layer. The second region 378 also can include a viewing window 388 that is illustrated as being rectangular, although other sizes and shapes are contemplated for he viewing window 388. The viewing window 388 can be integrated within the barrier 352, or can be coupled to respective surfaces of the barrier 352 to form a sealing engagement with the barrier 352. The barrier 352 generally allows personnel to view the contents situated within the interior volume of the 3D structure 354. Thus, the viewing window 388 can be formed out of plastics that allow visible light through.

As shown, the free end 362 also has a strip 390 having a coupling layer, which is coupled to and extends along a distal end of the free end 362. The free end 364 also has strips 392, 394, 396 that are coupled to and that extend along respective edges (or ends) of the free end 364. The strips 392, 394, 396 also have a coupling layer. The coupling layer can be embodied in many different forms. For example, the coupling layer can allow the respective component to be removably coupled to another components. Thus, the coupling layer can include a magnet, a hook and loop fastener, a different fastener (e.g., a bolt and a nut), etc. The coupling layer can also be configured to couple to components together. Thus, the coupling layer can be, for example, an adhesive layer that bonds to the other component.

FIG. 11 shows the isolation system 350 in the 3D configuration with the barrier 352 having the 3D structure 354. In some non-limiting examples, prior to the surgery the personnel can assemble the isolation system 350 from the 2D configuration of FIG. 10 to the 3D configuration of FIG. 11. For example, the coupling layers of respective strips of respective free ends can be coupled to the barrier 352, or other coupling layer (e.g., in the case of being removably coupled). Additionally, as shown, support beams 400, 401 can be inserted into respective sleeves to structurally support the 3D structure 354 of the barrier 352, which will be described in more detail below. In some non-limiting examples, the support beams 400, 401 can be coupled together so that the support beams 400, 401 are coupled together with another support beam (e.g., to define an arch, or u-shape). In particular, one end of a support beam can be coupled to an upper end of the support beam 400, and an opposing end of the support beam can be coupled to an upper end of the support beam 401. In this way, one contiguous support beam (a combination of the support beams 400, 401 and the another support beam) can span over multiple edges of the barrier 352 to help define the 3D structure of the barrier 352. In some cases, an additional sleeve can be coupled to the region 376 so that the another support beam of the contiguous support beam can be inserted through the additional sleeve.

In some non-limiting examples, while not illustrated in FIGS. 10 and 11, the isolation system 350 can also include arm ports, instrument ports, suction ports, etc. For example, the free ends 358, 362 can each include one or more arm ports. As another example, the region 376, and the free end 364 can each include instrument ports, and suction ports.

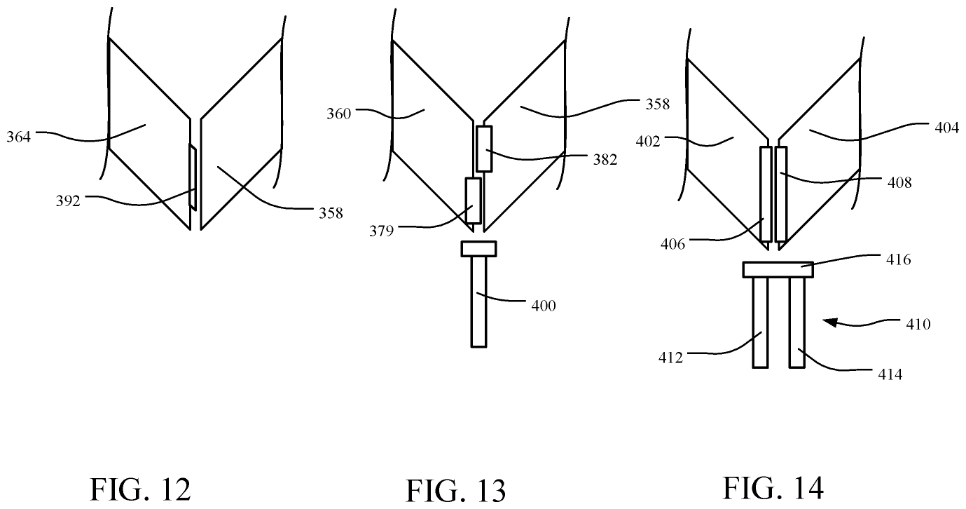

FIGS. 12-14 show different examples for how the 3D structure of the isolation system 350 can be realized. For example, FIG. 12 shows the strip 392 of the free end 364 being joined to the free end 358. In some non-limiting examples, respective edges of respective free ends can be aligned prior to the adjoining of the free ends 364, 358 with the coupling layer of the strip 392. FIG. 13 shows the sleeves 379, 382 of the respective free ends being aligned prior to insertion of the support beam 400. The support beam 400 is illustrated as having a pin-like structure, and is configured to be received through the sleeves 379, 382 to join the free ends 364, 358 together. FIG. 14 shows a different example of how sleeves and a support beam could be implemented. More specifically, FIG. 14 shows free ends 402, 404 of another barrier that have respective sleeves 406, 408. A support beam 410 having a first axial portion 412, a second axial portion 414 and a bridge 416 coupling together the axial portions 412,414. In some non-limiting examples, the sleeves 406, 408 can be aligned and the support beam 410 can join the free ends 402, 404 by the insertion of the first axial portion 412 into the sleeve 406 and the insertion of the second axial portion 414 into the sleeve 408.

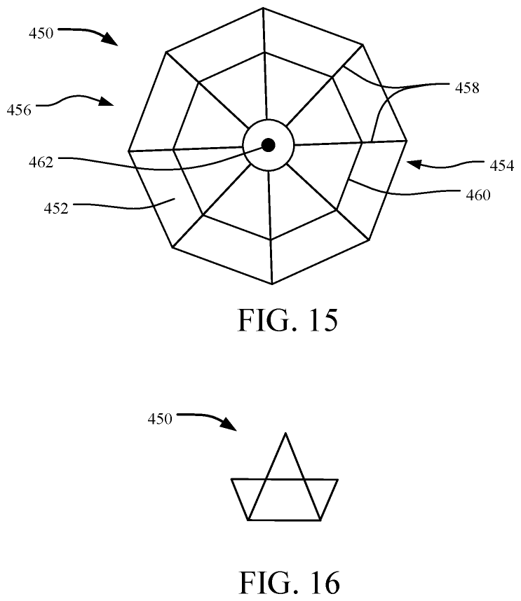

FIGS. 15-16 show an example of another isolation system 450. The isolation system 450 pertains to the previously described isolation systems and thus features of the other previously described isolation systems can be implemented with this isolation system and vice versa. The isolation system 450 has a barrier 452 defining a 3D structure 454. As shown, the isolation system 450 also can include a support structure 456 that helps to maintains the 3D structure 454 of the barrier 452. As shown, the support structure 456 can include a series of folds (or creases) 458, 460. The folds 458 are orientated along the axial axis 462 of the 3D structure 454, and extend from the axial axis 462 at different radial directions relative to each other. Although the folds 458 are equidistant to each other, in alternative non-limiting examples other separating distances are contemplated. Additionally, in some non-limiting examples, different numbers of folds 458 can be implemented other than the illustrated number of eight folds 458. The fold 460 is oriented around the axial axis 462 of the 3D structure 454, and can include a number of connected linear segments that intersect the folds 458 (and others). In other non-limiting examples, the fold 460 can be circumferentially oriented around the axial axis 462. Although a single fold 460 is illustrated, in alternative non-limiting examples, different numbers of folds 460 can be implemented. For example, multiple folds 460 and be oriented around the axial axis 462, and separated along the axial axis 462. Additionally, although the isolation system 450 is illustrated as having a generally conical shape, other shapes are contemplated with folds that help to define the 3D structure of the barrier. For example, in some non-limiting examples, the isolation system 450 can have a cylindrical shape.

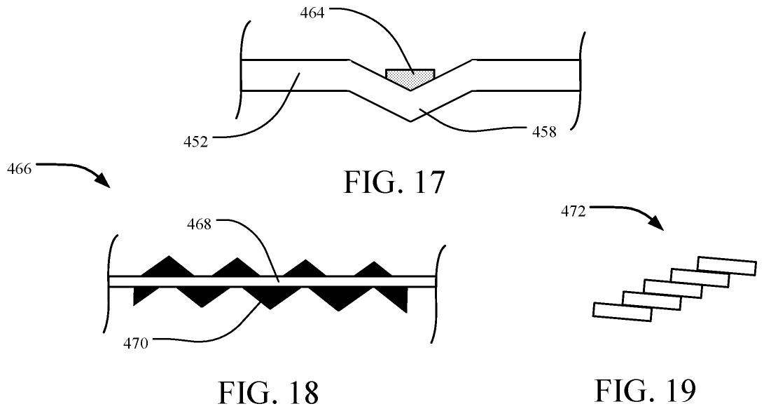

FIGS. 17-19 show different ways to define (or further define) the 3D structure 354 of the barrier 352. For example, FIG. 17 shows a support layer 464 coupled to an interior surface of a fold 458 of the barrier 452. The support layer 464 can extend along (a portion of) the entire fold 458. The support layer 464 can have various thicknesses, widths, lengths, shapes, etc. The support layer 464 can be rigid, semi-rigid, etc., and thus can be formed out of various materials (e.g., metals, plastics, etc.). In some non-limiting examples, the support layer 464 joins opposing portions of the fold 458 to prevent unfolding of the fold 458. Thus, the support layer 464 can be an adhesive, a polymer, etc. In alternative non-limiting examples, the fold 458 can be omitted and the support layer 464 can help to define the 3D structure 354.

FIGS. 18 and 19 shows different examples of pleats that can be used to help maintain a 3D structure of a barrier. For example, FIG. 18 shows a pleat 466 that is implemented as being a cartridge pleat. The pleat 466 has at least one thread 468 that is received through a barrier 470 that has been folded in an accordion-like fashion. The thread 468 can be stitched, knotted, etc., on opposing ends to maintain the folding pattern of the barrier 470. In some cases, the thread 468 can be elastic. FIG. 19 shows another example of a pleat 472, which is implemented as being a box pleat that has multiple layers of fabric to form the pleat 472. The pleats 466, 472 are intended only to be examples, and other pleats can be utilized. Additionally, the pleats as previously described can be used to further help to maintain the 3D structure of the barrier. For example, if the 3D structure of the barrier is curved, pleats can be coaxially oriented around an axial axis of the curved 3D structure. This can provide circumferential rigidity for the curved 3D structure.

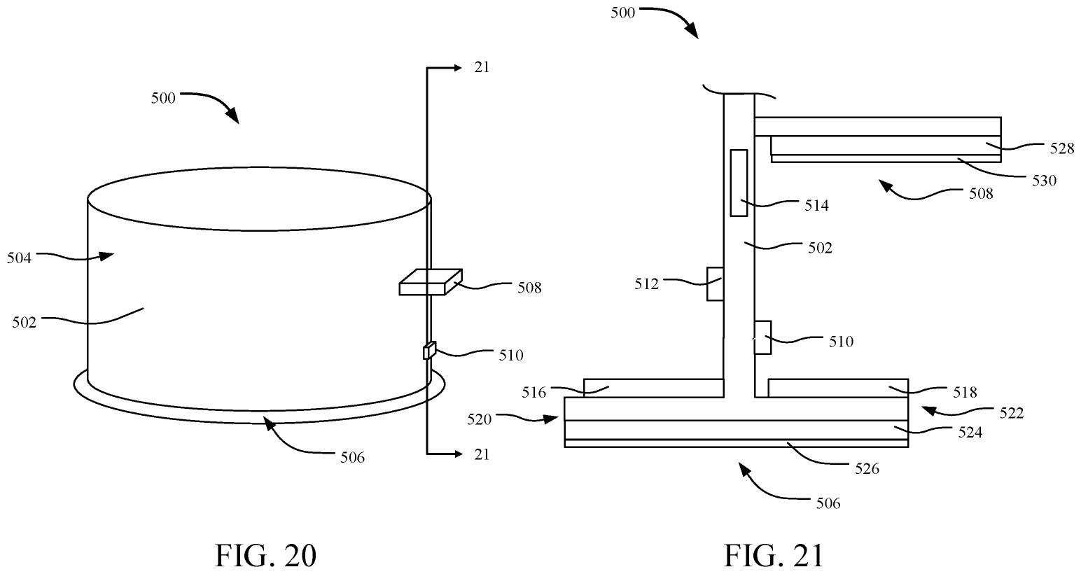

FIGS. 20 and 21 show an example of another isolation system 500. The isolation system 500 pertains to the previously described isolation systems and thus features of the other previously described isolation systems can be implemented with this isolation system and vice versa. The isolation system 500 is similar to and can contain similar components to that of the previously described isolation systems. The isolation system 500 can include a barrier 502 having a 3D structure 504, a flange 506, a tab 508, weights 510, 512, 514, and reinforcing members 516, 518. Similarly to the previously described flanges, the flange 506 extends around the entire periphery of the 3D structure 504, however in alternative configurations the flange 506 can be separated into regions separated by gaps having no flanges. As shown, the flange 504 has one end 520 that extends into the interior volume of the 3D structure 504 and an opposing end 522 that extends into the exterior space. The flange also has a coupling layer implemented as an adhesive layer 524 coupled to a lower surface of the flange 506, and an adhesive backing 526 coupled to the adhesive layer 524.

The isolation system 500 can include weights 510, 512, 514 that can help maintain the 3D structure 504. As shown, the weight 510 is coupled to the exterior surface of the barrier 502, the weight 512 is coupled to the interior surface of the barrier 502, and the weight 514 is integrated within the barrier 502. Although the illustrated non-limiting example of FIGS. 20 and 21 shows three weights, any number of weights can be utilized to realize the 3D structure 504. In some non-limiting examples, reinforcing members can be coupled to various portions of the flanges (or integrated within a flange) to provide structural reinforcement for the flange, and to provide weight to the flange. For example, the reinforcing member 516 is coupled to the upper surface of the end 520 of the flange 506, and the reinforcing member 518 is coupled to the upper surface of the end 522 of the flange 506. The reinforcing members 516, 518 can extend along various portions of the flange 506 (e.g., the entire circumferential distance or portions of the circumferential distance of the 3D structure 504).

The illustrated non-limiting example of FIGS. 20 and 21, the tab 508 is coupled to the exterior surface of the barrier 502, however in alternative non-limiting examples the tab 508 (or multiple tabs) can be coupled to the interior surface of the barrier 502 (and have coupling layers). The tab 508 also can include a coupling layer implemented as an adhesive layer 528 with an adhesive backing 530.

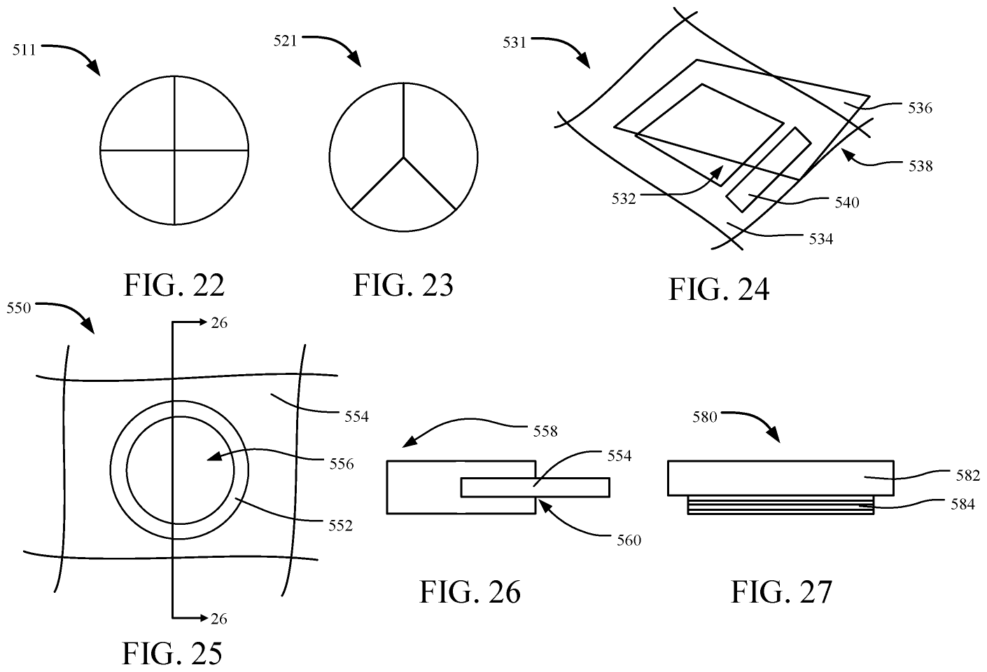

FIGS. 22-26 show specific examples of arm ports for the barrier of any of the isolation systems. For example, FIG. 22 shows an example of an arm port that is implemented as a valve 511 having four flaps that join together to generate a seal. FIG. 23 shows an example of another arm port implemented as another valve 521 having three flaps that join together to generate a seal. FIG. 24 shows an example of another arm port 531 having a hole 532 through a barrier 534. The arm port 531 also can include a flap 536 that is coupled to the barrier 502 and that spans across the hole 532 when in sealing engagement with the barrier 534. The flap 536 can include a coupling layer implemented as a magnetic strip 538, and the barrier 534 also can include a coupling layer also implemented as a magnetic strip 540. The flap 536 is removably coupled to the barrier 502 by the engagement between the magnetic strips 538, 540.

FIG. 25 shows an example of another arm port 550. The arm port 550 can include a rigid attachment 552 that is coupled to a barrier 554. The rigid attachment 552 has a hole 556 therethrough that allows access into the interior volume defined by the barrier 554. The rigid attachment 552 also can include an engagement feature 558 that allows components to be removably coupled to the rigid attachment 552. For example, the engagement feature 558 can be (or include) a magnet, a hook and loop fastener, threads, etc. As shown in FIG. 26, the rigid attachment 552 can include a slot 560 that receives a peripheral edge of a hole directed into the barrier 554.

FIG. 27 shows an example of a plug 580 that can be interfaced with an arm port (e.g., to seal the arm port). The plug 580 has a body 582 and a protrusion 584 extending from the body 582 that is threaded. The threads of the body 582 correspond to threads of the rigid attachment 552 (e.g., the engagement feature 558). In alternative non-limiting examples, the protrusion can include other coupling components that correspond to the engagement feature 558. Additionally, in other configurations, the protrusion 584 can be omitted and rather the body 582 can include the threads (or other coupling components).

Although FIGS. 22-27 have been described as being arm ports, in other non-limiting examples and as described above, the configurations of the arm ports can be implemented accordingly (e.g., decreased in size) for instrument ports, fluid ports, suction ports, etc.

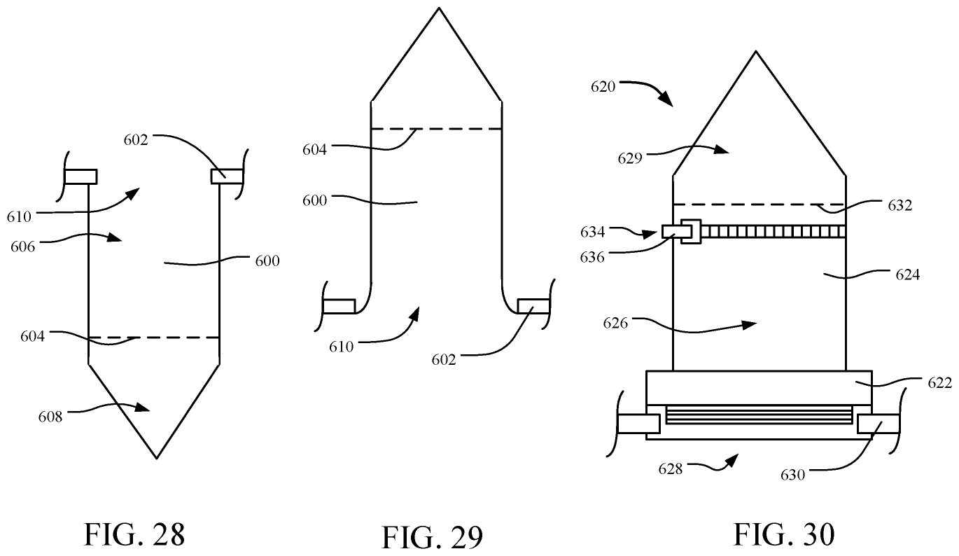

FIG. 28 shows an example of an arm sleeve 600 that can be implemented on any of the previous isolation systems. The arm sleeve 600 is integrally formed with and extends away from the barrier 602 in the first configuration of FIG. 28. In some non-limiting examples, and as illustrated, the arm sleeve 600 can include a region of material weakness implemented as a perforation 604, which is located on the arm portion 606 of the sleeve 600. However, in alternative non-limiting examples, the region of material weakness (or perforation 604) can be located on the hand portion 608 of the sleeve 600. Although the hand portion 608 is illustrated as having a generally decreasing cross-sectional area away from the barrier 602 in the first configuration, in other configurations, the hand portion 608 can contour the hand of the subject (e.g., the hand portion 608 can be shaped as a glove).

FIG. 29 shows the arm sleeve 600 in the second configuration. For example, prior to when personnel proceeds with the procedure, the arm sleeve 600 is typically in the first configurations being in the exterior space relative to the barrier 602. Then, as desired the arm sleeve 600 can be inverted an pushed into the interior volume of the barrier 602 to conduct the procedure while not directly contacting the subject (or having another layer of protection from a patient). In some cases, one hand of another arm sleeve can grasp the hand portion 608 and tear the hand portion 608 from the arm portion 606 along the perforation 604. This can be advantageous in that during the procedure personnel needs better gripping ability (e.g., to grasp instruments) and the perforation allows for easy removal of a portion of the arm sleeve 600. In some cases, the region of material weakness can be positioned at different locations of the arm sleeve 600, such as within the hand portion 608 of the arm sleeve 600 (or closer to the barrier 602). Additionally, as described previously other components can be included with the arm sleeves 600 (and other arm sleeves), such as a valve that spans across the opening 610 of the barrier 602. In some non-limiting examples, the perforation 604 can be replaced (or added) with a different removably coupled configuration (e.g., hook and loop fasteners, magnets, fasteners, etc.).

FIG. 30 shows another example of an arm sleeve 620 that can be implemented with any of the isolation systems. The arm sleeve 620 can include a rigid cuff 622 and a sleeve 624 coupled to the rigid cuff 622 at one end. Similarly to the arm sleeve 600, the sleeve 624 also can include an arm portion 626 and a hand portion 629. The rigid cuff 622 of the arm sleeve 620 is configured to be removably coupled to the previously described arm ports. For example, as shown in FIG. 30 the rigid cuff 622 is threadingly engaged with a rigid attachment 628 that is coupled to a barrier 630 having a hole therethrough (e.g., similarly to the rigid attachment 552). This threading engagement (or other removably coupled configuration) allows for a sealing engagement.

In some non-limiting examples, the arm sleeve 620 also can include a region of material weakness implemented as a perforation 632, and a clasp locker 634 (e.g., a zipper) having a slider 636. In a first configuration, similarly to the arm sleeve 600, the sleeve 624 extends away from the barrier 630 and into the exterior space (e.g., an inverted configuration to the configuration of FIG. 24). In the first configuration, the slider 636 is positioned in the internal volume defined by the sleeve 624. Then, prior to conducting a procedure, the sleeve 624 of the arm sleeve 620 can be inverted and pushed into the interior volume of the barrier 630 to place the arm sleeve 620 in a second configuration. As desired, the slider 636 can be grasped when the arm sleeve 620 is in the inverted second configuration to decouple portions of the sleeve 624.

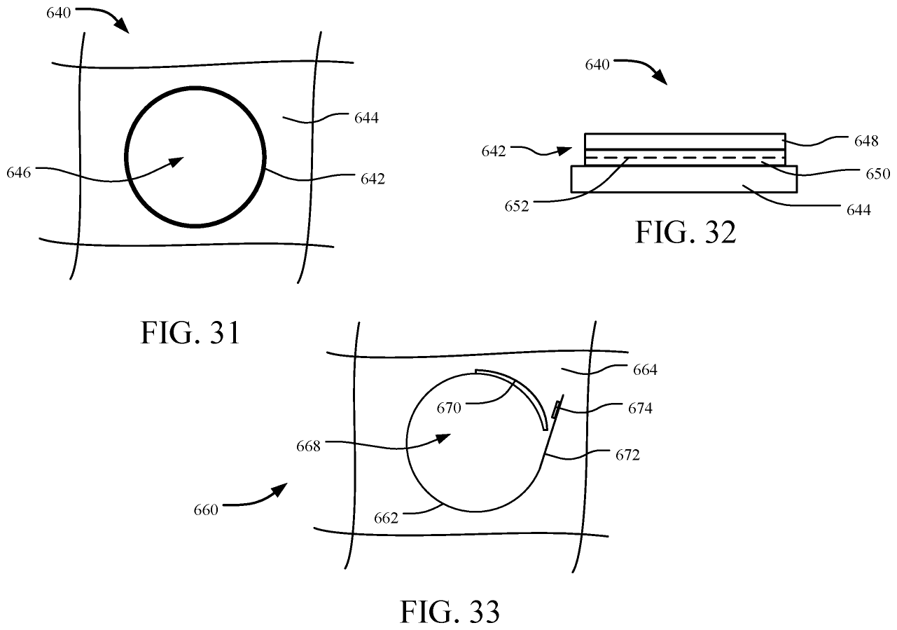

FIGS. 31-33 show different examples of instrument attachments that can be implemented with any of the isolation systems. For example, FIGS. 31 and 32 show an example of an instrument attachment 640 having a ring 642 that extends upwardly from a barrier 644 having a hole 646 therethrough. The ring 642 has an elastic portion 648, and a non-elastic portion 650 having a region of material weakness implemented as a perforation 652. In some non-limiting examples, the perforation 652 allows easy removal of the barrier 644 from the instrument (e.g., a microscope), followed by subsequent removal of the elastic portion 648. The elastic portion 648 can be expanded to be retractably secured around a component of the instrument. In some non-limiting examples, the non-elastic portion 650 can be elastic.

FIG. 33 shows an example of another instrument attachment 660. The instrument attachment 660 can include a cuff 662 upwardly extending from the exterior surface of a barrier 664 around a hole 668 of the barrier 664. The cuff 662 has a first coupling layer 670, and a free end 672 with a second coupling layer 674. The second coupling layer 674 of the free end 672 can be removably coupled (or coupled) to the first coupling layer 670 (or the cuff itself 662) at different locations to adjust the size of the circumference of the cuff 662 so that the cuff 662 can be securely engaged with a portion of an instrument (e.g., a microscope).

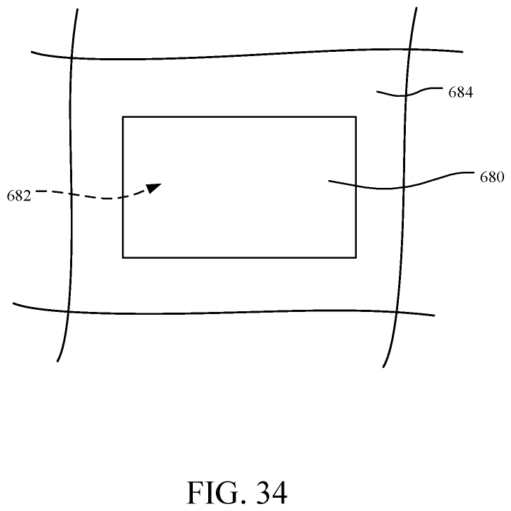

FIG. 34 shows an example of a viewing window 680 that can be implemented with any of the isolation system. The viewing window 680 can be formed of various materials with various optical properties, so that light from the external space can penetrate through the viewing window 680. The viewing window 680 spans across a hole 682 directed through a barrier 684, and thus provides a seal with the barrier 684. As such, the viewing window 680 can be coupled to one of the surfaces of the barrier 684 or integrated within the barrier 684.

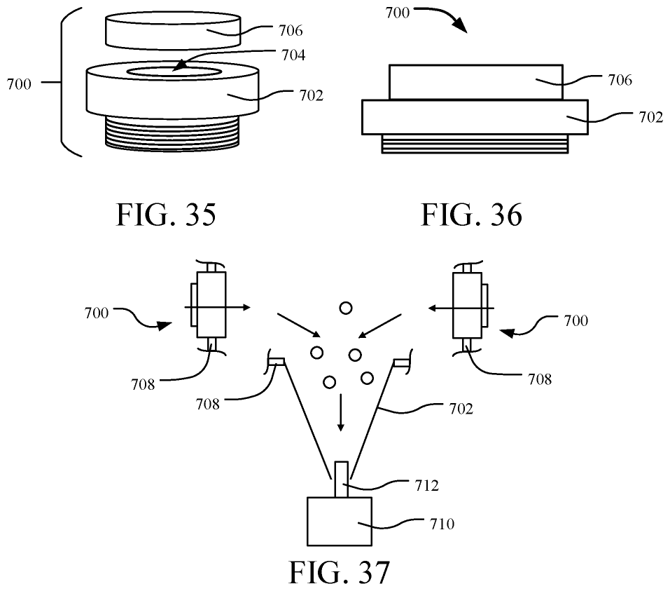

FIGS. 35 and 36 show an example of a fluid port 700 that can be implemented with any of the isolation systems. The fluid port 700 can include a rigid body 702 having a hole 704 therethrough, and a filter 706 coupled to the upper surface of the rigid body 702. The filter 706 is shown extending across the hole 704, although the filter 706 can be coupled to the rigid body 702 to generally be in sealing engagement with the rigid body702 (e.g., the filter 706 being disposed in the hole 704). The fluid port 700 has threads and is configured to be threadingly engaged with a rigid attachment as previously described. However, in alternative configurations, the fluid port 700 can simply be a hole through the barrier, and can include a filter that extends across or is received in the hole (e.g., in sealing engagement). The filter 706 can have pores that are sized to block tissue particulates, pathogens, or both. In some non-limiting examples, the filter 706 (or other filters) engaged with a barrier allows fluid (e.g., air) contained in the interior volume to flow out through the filter and into the exterior space, and allows fluid containing in the exterior space to flow through the filter and into the exterior space. The flow of fluid into and out of the interior volume via the filters allows the interior volume to be at a substantially constant volume even when fluid is removed from the interior volume (e.g., with a suction source). The filter configuration can be especially advantageous in that the filters allow for a substantially low resistance fluid flow path, and thus prevents fluid from entering other undesirable ports or structures (e.g., under the flange, the instrument attachment, etc.).

For example, FIG. 37 shows specific locations of fluid ports having filters on an isolation system. The fluid ports 700 are shown being interfaced with a barrier 708 at different locations. As shown, a suction source 710 having a probe 712 that is interfaced with a suction bag 714 that is integrally formed with (or coupled to) the barrier 708 (although other configurations of the suction bag are possible, such as using the previously described rigid cuffs, attachments, etc.). The positioning of the fluid ports 700 having filters provides fluid flow that drives aerosols and other particulates to be suctioned by the suction source 710. In some non-limiting examples, a fluid pump (e.g., an air pump) can be interfaced with the fluid port 700 (or other fluid port) to drive fluid into the interior volume of the 3D structure. In some non-limiting examples, the net flux of fluid (e.g., air) into the interior volume of the 3D structure can be substantially zero (e.g., so as to prevent fluctuations in the value of the interior volume), which can be accomplished by forcing the total suction rate out of the isolation system to be equal to the forced air into the isolation system.

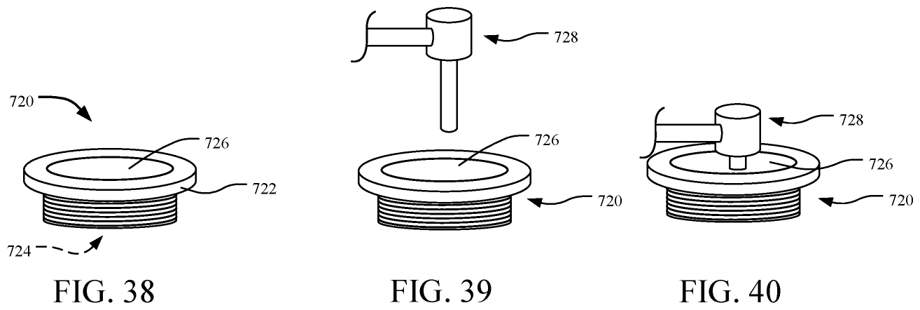

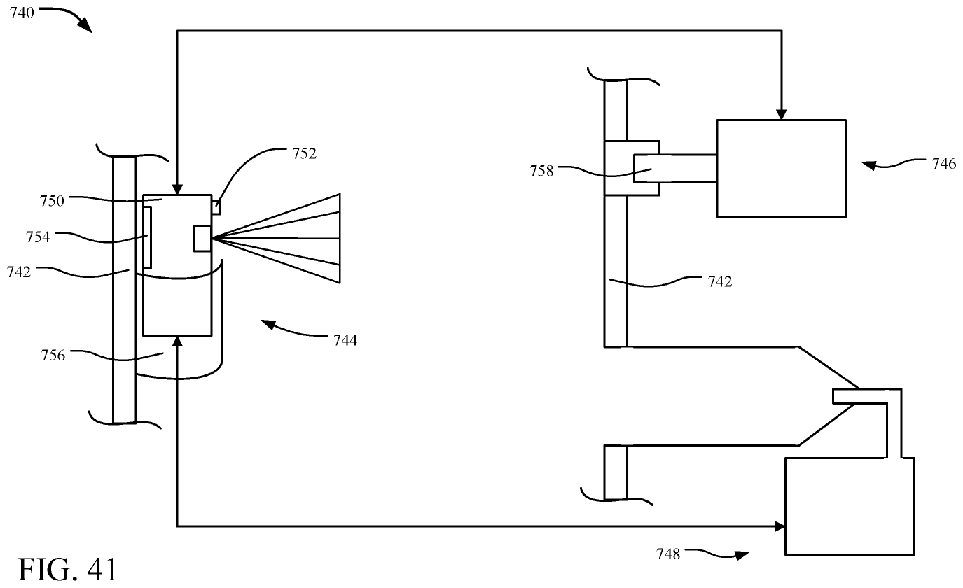

FIGS. 38-40 show an example of a suction port 720 that can be used with any of the isolation systems. The suction port 720 can include a rigid body 722 having a hole 724 therethrough, and a membrane 726 coupled to the rigid body 722 and extending across the hole 724. The suction port 720 can include threads to threadingly engage with a rigid attachment (previously described). In some non-limiting examples, the rigid body 722 can be omitted and the membrane 726 can be coupled to or integrated with a barrier. FIG. 39 shows an example of suction probe 728 of a suction system prior to being interfaced with the suction port 720. FIG. 40 shows the suction probe 728 interfacing with the suction port 720. More specifically, the suction probe 728 penetrates the membrane 726 and is inserted into the interior volume of the barrier. By penetrating the membrane 726 (with the suction probe 728) a hole in the membrane forms that has edges that sealingly engage with the probe 728. Once the procedure is completed, the suction probe 728 can be removed from the membrane 726, and can be cleaned and reused again (or disposed of properly).

FIG. 41 show an example of another isolation system 740, which is similar to the other isolation systems of this disclosure, and which components of the isolation system 740 can be utilized with the other isolation systems of this disclosure (and vice versa). The isolation system 740 can include a barrier 742, a detection system 744, a HEPA filtration system 746, and a suction source 748. The detection system 744 can include a housing 750, a light source 752, an image sensor 754, and other computing components (not shown). The components of the detection system 744 are integrated within the housing 750, and the housing 750 is secured in a sleeve 756 within the interior volume of the barrier 742.

As shown, the HEPA filtration system 746 has tubing 758 that interfaces with a port (previously described) of the barrier 742. Similarly, the suction source 748 also integrates with a port located in a suction bag coupled to the barrier 742. In some non-limiting examples, the detection system 744 can be in communication with the HEPA filtration system 746 and the suction source 748. In some non-limiting examples, the detection system 744 is configured to acquire imaging data and to extract particle density data from the imaging data, which can be comparted to a threshold value, and if the particle density data exceeds the threshold the detection system 744 can cause the HEPA filtration system 746, or the suction source 748 to begin providing a suction (e.g., to activate) or to increase the amount of suction (e.g., increase volume per unit time).

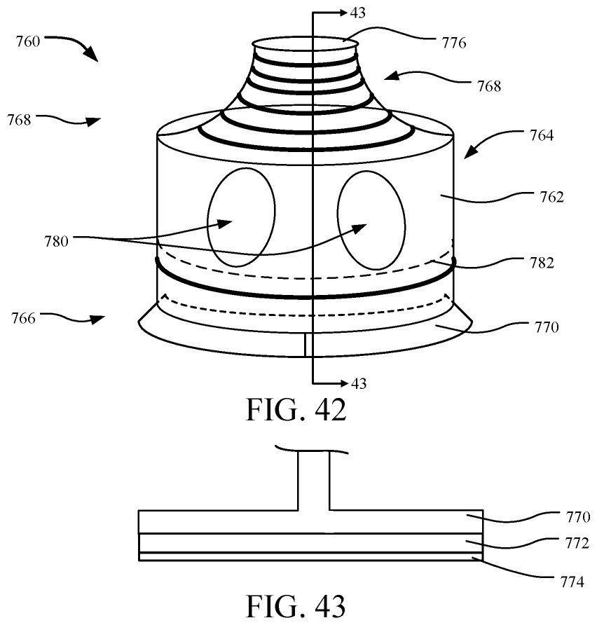

FIGS. 42 and 43 show another example of an isolation system 760. The isolation system 760 is similar to the previous isolation systems, and components from the other isolation systems of the disclosure can be utilized in the isolation system 760 (and vice versa). The isolation system 760 can include a barrier 762 defining a 3D structure 764 having two sections 766, 768 that can be coupled to each other. The lower section 766 can include a flange 770 having an adhesive layer 772 and an adhesive backing 774 coupled thereto. The upper section 768 can include an instrument attachment 776, a scaffold 778, and arm ports 780. As shown, the dotted line 782, which separates the sections 766, 768 shows the location of a coupling feature that joins the sections together. The coupling feature can be implemented in many different ways, including adhesive strips, hook and loop fasteners, snap fitted connections (e.g., a rigid protrusion(s) snap fitted in a rigid slot(s) to join the components together), etc. The coupling feature advantageously allows the lower section 766 to be installed first. For example, the flange 770 of the lower section 766 can be joined to a patient (or other structure) with the adhesive layer 772. Then, other steps of a procedure that are less likely to generate aerosols or tissue particulates can be completed with the lower section766 installed (e.g., creating an incision). Then, prior to steps of the procedure that are likely to generate aerosols or tissue particulates (e.g., bone drilling) the upper section 768 can be coupled to the lower section 766. With the upper section 768 secured, the steps of the procedure that likely generate aerosols or tissue particulates can be completed.

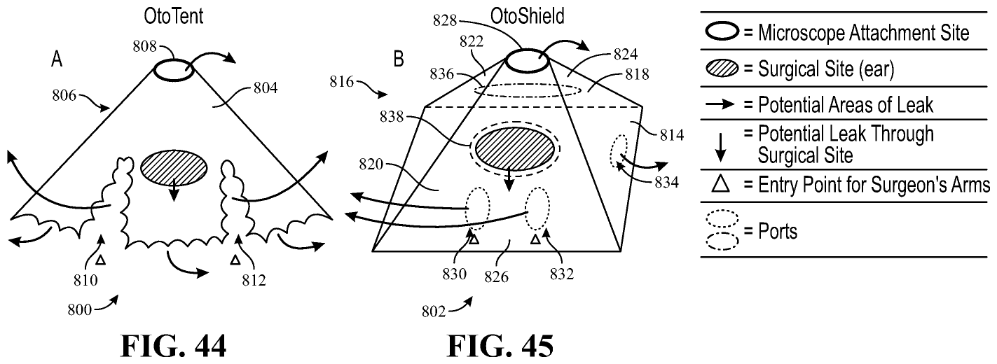

FIG. 44 shows a front view of another isolation system 800, while FIG. 45 shows a front perspective view of another isolation system 802. Each of the previously described isolation systems pertain to the isolation systems 800, 802. As shown in FIG. 44, the isolation system can include a barrier 804 that defines a 3D structure 806, a microscope attachment 808 coupled to an end of the barrier 804, and arm ports 810, 812. Each arm port 810, 812 can include a cutout that is directed from an end of the barrier 804 (e.g., opposite the end having the microscope attachment 808) and towards the opposing end of the barrier 804 (e.g., that has the microscope attachment 808). For installation, the microscope attachment 808 is coupled to a microscope and the barrier 804 is positioned to enclose a tissue site (e.g., an ear).

The isolation system 802 can include a barrier 814 that defines a 3D structure 816 and which has a plurality of faces 818, 820, 822, 824, 826. Each of the faces 818, 820, 822, 824, 826 are joined together to define the 3D structure 816, which in this case is a square pyramid although other 3D shapes are contemplated. The bottom face 826 is coupled to each of the faces 818, 820, 822, 824, and can include a coupling layer (e.g., an adhesive) situated on an exterior surface. The isolation system 802 can also include a microscope attachment 828, ports 830, 832, 834, 836, 838, with the ports 830, 832 being directed into the face 820, with the port 834 being directed into the face 818, with the port 836 being directed into the face 824, and with the port 838 being directed into the face 826.

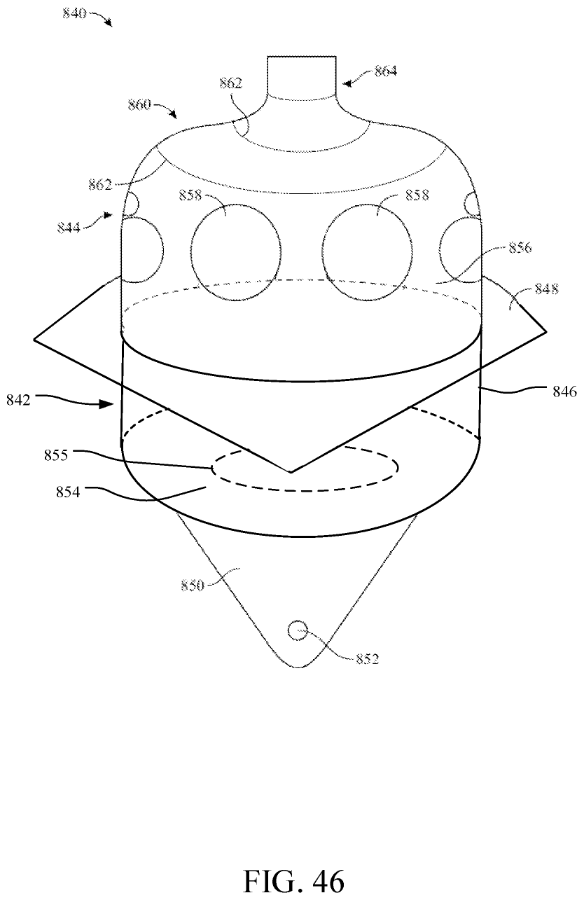

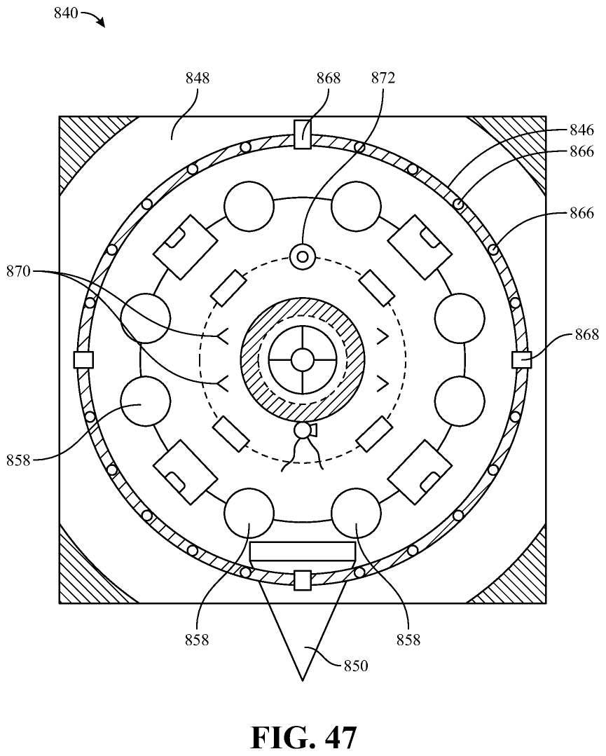

FIG. 46 shows a front view of another isolation system 840, while FIG. 47 shows a top view of the isolation system 840. The isolation system 840 pertains to the previously described isolation systems (e.g., features of other previously described isolation systems can be implemented with this isolation system and vice versa). As shown in FIG. 46, the isolation system 840 can be formed out of a two part construction, with a bottom section 842 that interfaces with a top section 844. The bottom section 842 can include a barrier 846, and a flap 848 that is coupled to the barrier 846 and that peripherally extends around the barrier 846. The barrier 846 defines a 3D structure that can enclose an object within its interior volume. Although not illustrated, a top of the bottom section 842 is opened (e.g., exposed to the ambient environment) prior to being interfaced with the top section 844. In some cases, the flap 848 can partially extend around the barrier 846, while in other cases, the flap 848 can entirely extend around the barrier 846. A lower surface of the flap 848 can have a coupling layer (e.g., an adhesive layer with a backing) that can be secured to a patient (or a structure). This coupling layer can be located on a lower, exterior surface of the flap 848. In some non-limiting examples, the bottom section 842 can include a suction bag 850 coupled to the barrier 846, which can include a suction port 852 to receive a suction source (e.g., to drain fluid from the interior volume of the isolation system 840). In some cases, the suction bag 850 can be internally located within the barrier 846, while in other cases, the suction bag 850 can be integrally formed with the barrier 846 so that the periphery of the barrier 846 narrows with the suction bag 850. In some cases, the barrier 846 can have a bottom 854 and a hole 855 that can be directed though the bottom 854. The bottom 854 can be flat, and can include a coupling layer situated on an exterior surface of the bottom 854. The hole 855 is situated to receive a tissue site (or a laboratory sample), and the coupling layer of the bottom is coupled to the patient (or a structure) to restrict relative movement between the barrier 846 and the patient (or the structure). The hole 855 can have various shapes. For example, the hole and be a u-shaped cutout, a circular cutout, an oval cutout, etc.

The top section 844 can also include a barrier 856 that defines a 3D structure that can enclose an object within its interior volume. Similarly to the bottom section 842, a bottom of the top section 844 is opened (e.g., exposed to the ambient environment) prior to being interfaced with the bottom section 842. As shown in FIG. 46, the top section 844 can include a plurality of arm ports 858 that are directed into the barrier 856, a scaffold 860 including a plurality of rings 862, and a microscope attachment 864. While four pairs of arm ports 858 are illustrated, with a first pair being situated on a first end of the barrier 856, with a second pair being situated on a second end of the barrier 856 and opposite the first end of the barrier 856, with a third pair being situated adjacent the first and second pairs, and with a fourth pair being situated opposite the third pair, the barrier 856 can include different numbers of arm ports 858. As shown in FIG. 46, the scaffold 860 is coupled to the barrier 856, which helps to define the 3D structure of the barrier 856 (and thus the interior volume defined by the 3D structure). The scaffold 860 can include rings 862 that are separated from each other, and which can be concentric to each other (e.g., along the length of the top section 844). While there are two rings 862 illustrated, the scaffold 860 can include other numbers of rings. In addition, the rings 862 can have other shapes, such as enclosed bands.

Regarding installation, with the bottom 854 coupled to the patient (or a structure) the flap 848 is radially drawn away from the barrier 846 (e.g., folded away) and is pulled (in tension) to couple the coupling layer of the flap 848 to the patient (or a structure). In this way, with the flap 848 pulled and secured in tension, the bottom section 842 is reinforced and further restricted from moving relative to the patient (or structure). Then, the top section 844 is placed into engagement with the lower section 842, or in other words the sections 842, 844 are coupled together. In some cases, the barrier 856 of the top section 844 can be slid over the barrier 846 of the bottom section 842 (or vice versa). Then, the barriers 846, 856 can be coupled together (e.g., with fasteners, such as adhesives, etc.). For example, the inner (or outer) surface of the barrier 846 can have a coupling layer, and the inner (or outer) surface of the barrier 856 can have a coupling layer. In some cases, the barriers 846, 856 can be snap-fitted together (e.g., when ends of each barrier 846, 856 have a rigid coupling). Then, the microscope attachment 864 can be coupled to a microscope and the procedure (or observation of a sample) can proceed.

In some configurations, while the upper section 844 and the lower section 842 are illustrated as having particular components, in other configurations, each of the sections can have the same components. For example, the lower section 842 can have a scaffold, arm ports, etc., while the upper section 844 can include a flap, a suction bag, a suction port, etc. In addition, while the barrier 846 of the bottom section 842 and the barrier 856 of the top section 844 are illustrated as being cylindrical, in other configurations, the barriers 846, 856 can have a similar shape, but with the similar shape differing (e.g., conical).

In some non-limiting examples, the isolation system 840 can include a plurality of weights 866 that are coupled to the barrier 846 and are situated around the periphery of the barrier 846, and a plurality of tabs 868 that are also coupled to the barrier 846 and are also situated around the periphery of the barrier 846. Each tab 868 can have a coupling layer positioned on its lower exterior surface to be secured to a patient (or a structure). As shown, in some cases, the barrier 856 can include a plurality of pleats 870, and an instrument port 872 directed into the barrier 856. Each pleat 870 can extend in an axial direction along the barrier 856 (e.g., in a direction from the free end of the barrier 856 to the microscope attachment 864).

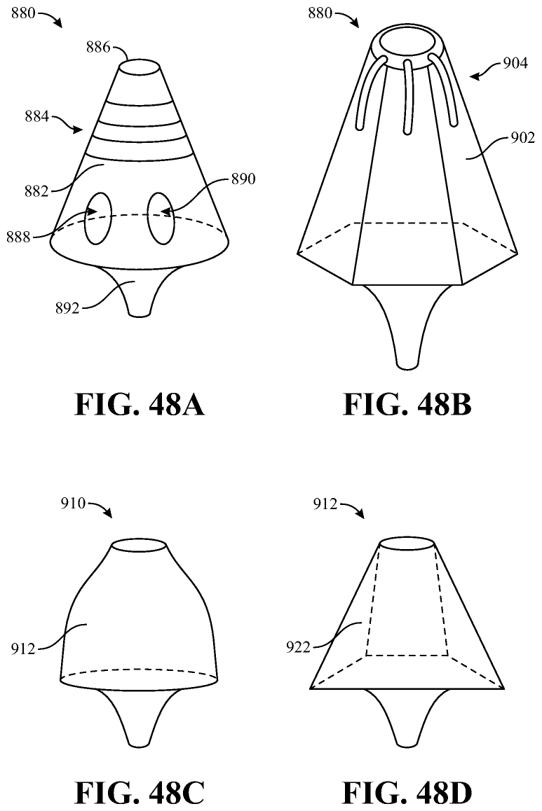

FIG. 48A shows a front view of another isolation system 880, which pertains to the previously described isolation systems (e.g., features of other previously described isolation systems can be implemented with this isolation system and vice versa). The isolation system 880 can include a barrier 882, a scaffold 884 coupled to the barrier 882, a microscope attachment 886, arm ports 888, 890, and a drainage bag 892 (or in other words a suction bag). As shown, the barrier 882 defines a 3D structure, which is illustrated as having a conical shape (e.g., a frusto-conical shape). The scaffold 884 can reinforce (or define) the 3D structure. For example, as shown, the scaffold 884 can include a plurality of rings that are separated from each other, and which extend along the height of the isolation system 880. In some cases, and as shown, the rings are concentrically oriented relative to each other (e.g., when viewed from a top view). In some non-limiting examples, the drainage bag 892 can be integrally formed with the barrier 882. In other words, the drainage bag 892 can be inbuilt with the barrier 882. In this way, the bottom of the barrier 882 can be enclosed by the drainage bag 892. In some cases, the walls of the barrier 882 transition to the drainage bag 892, and the drainage bag 892 narrows downwardly to an end. Situated at the end of the drainage bag 892 is a drainage port 894 (e.g., to receive a drainage source, a suction source, etc.). In some configurations, the drainage bag 892 can be coupled to the barrier 882 (e.g., rather than integrally formed with the bag 892) and the drainage bag 892 can be formed out of a different material than the barrier 882. For example, the drainage bag 892 can be formed out of a more elastic material than the barrier 882.

FIG. 48B shows a front view of another isolation system 900, which pertains to the previously described isolation systems (e.g., features of other previously described isolation systems can be implemented with this isolation system and vice versa). The isolation system 900 also can include a barrier 902, a scaffold 904 coupled to the barrier 902, a microscope attachment, and a drainage bag. The barrier 902 also defines a 3D structure, which in this case a pyramid (e.g., an octagonal pyramid). The barrier 902 has a number of faces that collectively define a multifaceted configuration. The scaffold 904 can include a ring, and can include a number of arms that radially emanate away from the ring and along a respective face of the barrier 902.

FIG. 48C shows a front view of another isolation system 910, which pertains to the previously described isolation systems (e.g., features of other previously described isolation systems can be implemented with this isolation system and vice versa). The isolation system 910 also can include a barrier 912, a scaffold coupled to the barrier 912, a microscope attachment, and a drainage bag. The barrier 912 also defines a 3D structure, which in this case spherical (e.g., a hemisphere).

FIG. 48C shows a front view of another isolation system 920, which pertains to the previously described isolation systems (e.g., features of other previously described isolation systems can be implemented with this isolation system and vice versa). The isolation system 920 also can include a barrier 922, a scaffold coupled to the barrier 922, a microscope attachment, and a drainage bag. The barrier 922 also defines a 3D structure, which in this case is a pyramid (e.g., a rectangular, or square pyramid).

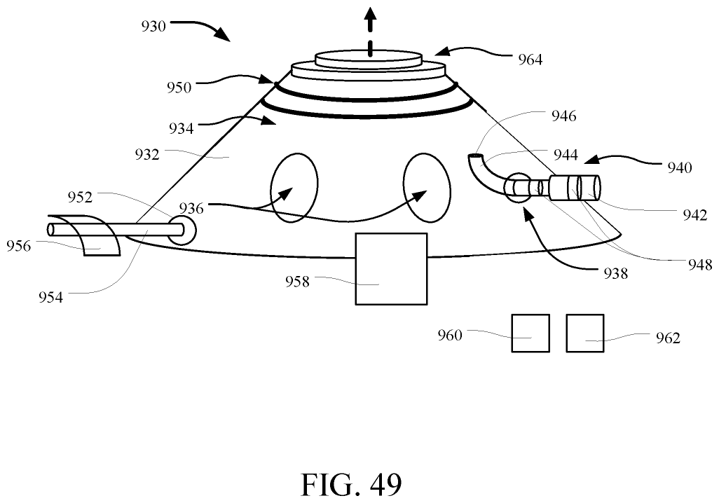

FIG. 49 shows an example of an isolation system 930, which pertains to the previously described isolation systems (e.g., features of other previously described isolation systems can be implemented with this isolation system and vice versa). The isolation system 930 can include a barrier 932 having a 3D structure 934 defining an interior volume of the barrier 932, arm ports 936, a fluid port 938, and a suction attachment 940. The fluid port 938 can be structured in many different ways, but generally allows fluid (e.g., air) communication between the interior volume of the barrier 932 and the exterior space that surrounds the barrier 932. The suction attachment 940 can be inserted into the fluid port 938, and in some cases can interface with the fluid port 938 to be in sealing engagement with the fluid port 938. As shown, the suction attachment 940 has a rigid portion 942, and a tubular portion 944 that is coupled to the rigid portion 944. The rigid portion 942 can be attached to a previously existing suction system (e.g., a suction probe, or a tube of a suction system that is located within the operating room). In some cases, the rigid portion 942 can be omitted so that the tubular portion interfaces with an existing suction system.

The tubular portion 944 can be curved and an end of the tubular portion 944 defines a fluid input 946 (e.g., an air input). By manipulating the suction attachment 940, the location, orientation, etc., of the fluid input 946 can be positioned as desired (e.g., how far the fluid input 946 extends into the interior volume of the barrier 932 and how the fluid input 946 is oriented, such as by rotating the tubular portion 944). For example, in some cases, the suction attachment 940 can be advanced into the interior volume to translate the fluid input 946. In operation, the fluid (e.g., air) enters the fluid input 946 is directed through the tubular portion 944, through the rigid portion 942, and is vacated out into the suction system (e.g., as appropriately attached). As shown, a filter 948 can be located within the tubular portion 944, and the rigid portion 942. The filters 948 can be high-efficiency particulate air (“HEPA”) filters. In some cases, the suction attachment 940 can be secured to a location with fasteners (e.g., adhesive butterfly strips, clips, hook and loop fasteners, etc.).

As shown in FIG. 49 and similarly to the other isolation systems previously described, the isolation system 930 can include a scaffold 950 coupled to the barrier 932, a fluid port 952, a tube 954 inserted through the fluid port 952, a strip of material 956 coupled to the tube 954, a drainage bag 958 coupled to and in fluid communication with the barrier 932, strips of material 960, 962, and an instrument attachment 964 coupled to the barrier 932. One end of the tube 954 can be interfaced with a suction system (e.g., a tube of wall suction tube, such as from a HEPA filtering system) and an opposing end of the tube 954 can be inserted through the fluid port 952 and into the interior volume of the barrier 932. The tube 954 can be flexible and can be pushed further into the interior volume, pulled out of the interior volume, and reoriented (e.g., the portion outside of the interior volume) such as curved. In addition, the strip of material 956 can have a coupling layer on its lower surface on opposing ends of the strip of material 956 to couple the oriented tube 954 to the patient (or a structure). In some cases, rather or in addition to the strip of material 956, the isolation system 930 can include a fastener to fasten the tube 954 to a component (or the patient). Each of the strips of material 960, 962 also include a coupling layer on one of its sides to reinforce coupling of components. For example, the strip of material 960 can be coupled to the barrier 932 and can be coupled to the patient (or the structure) to further restrict relative movement between the barrier 932 and the patient (or the structure). In some non-limiting examples, the tube 954 can be integrally formed with the fluid port 952, or can be integrally formed with the barrier 932 (e.g., with the fluid port 952 omitted). In some non-limiting examples, the end of the tube 954 situated within the interior volume of the barrier 932 can be placed in close proximity to the aspiration site (e.g., less than or equal to 3 cm away from the aspiration site), such as a drilling site (e.g., the drill head). In some cases, a filter (e.g., the filter 948) can be situated within the tube 954.

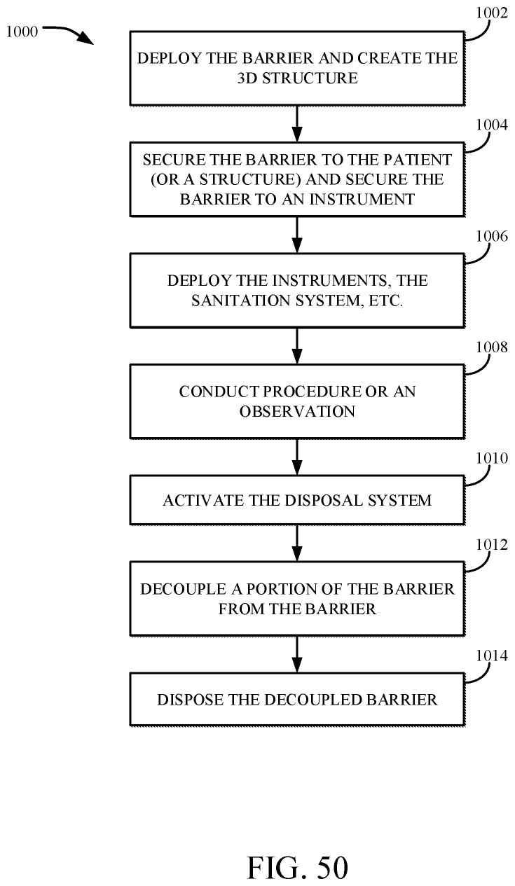

FIG. 50 shows a flowchart of a process 1000 for preparing a patient for a procedure, or a sample for observation. Any of the described isolation systems (and tents, barriers, etc., below) can be used to implement some or all of the portions of the process 1000, as appropriate.

At 1002, the process 1000 can include deploying the barrier of the isolation system to create a 3D structure. In some cases, this can include removing the isolation system from packaging (e.g., wrapping, such as plastic wrapping). In addition, this can include expanding the barrier from a compressed state to an expanded state. This can include expanding the scaffold of isolation system (e.g., coupled to the barrier) from a compressed state (e.g., a flat, or planar state) and to an expanded state (e.g., a 3D state). In some cases, this can include simultaneously expanding the scaffold and the barrier to define the 3D structure (e.g., such as when the scaffold is coupled to or integrally formed with the barrier). In some cases, this can include separating rings of the scaffold as the scaffold expands, or straightening an arm of the scaffold as the scaffold expands. In addition, this can include bending a beam of the scaffold to reorient the beam of the scaffold.

In some configurations, this can include installing the barrier, and installing the scaffold to the barrier. For example, this can include coupling two free ends the barrier together (e.g., with an adhesive). As another example, this can include aligning a sleeve of one free end of the barrier with another sleeve of another free end of the barrier, and inserting a support beam through both sleeves.

In some configurations, this can also include setting up the port(s) of the isolation system. For example, this can include opening a flap (e.g., the flap 536) of the instrument port and securing the flap open, removing a plug from a port, such as the arm port. In some cases, this can include threadingly disengaging the plug from a rigid attachment of the port. As another example, this can include opening a port, such as tearing a piece of material off of the isolation system to define the port, such as via an area of material weakness in the barrier.

In some non-limiting examples, this can also include setting up the arm sleeves of the isolation system. For example, this can include inverting the arm sleeve so the arm sleeve is forced into the interior volume of the barrier, coupling an arm sleeve to an arm port (e.g., threadingly engaging a rigid cuff of an arm sleeve to a rigid attachment of an arm port). In some cases, this can include removing a portion of the arm sleeve from the arm sleeve (e.g., along an area of material weakness of the arm sleeve). For example, a hand portion of the arm sleeve (e.g., which can be implemented as a glove) can be decoupled from the arm sleeve.

In some non-limiting examples, this can include coupling a suction system to a tube of the isolation system, coupling a suction system to a suction attachment of the isolation system. In addition, this can include inserting a tube through a port, advancing the tube through the port, retreating the tube through the port, changing the orientation of the tube within the interior volume of the barrier, etc.

In some non-limiting examples, the can include coupling a detection system to the barrier of the isolation system. For example, this can include coupling a light source to the barrier, coupling an imaging sensor to the barrier, etc.

At 1004, the process 1000 can include securing the barrier to the patient (or a structure), and securing the barrier to an instrument. For example, this can include coupling a flange of the barrier to a patient (or a structure) to enclose the patient (or a laboratory sample) within the volume of the barrier. In particular, an adhesive backing can be removed from the flange to expose the adhesive layer of the flange to couple the flange to the patient (or a structure). In some cases, this can include coupling a tab, which is coupled to the barrier, to a patient (or a structure). In addition, this can include coupling a peripheral flange (or in other words a flap) that is coupled to the exterior surface of the barrier to the patient (or a structure). For example, the peripheral flange can be pulled in tension and then coupled to the particular object or person. In some configurations, this can include advancing a lower edge of the barrier underneath the barrier, and coupling the lower edge to the patient or a structure. In some cases, this can include folding the barrier at a crease defined by the advancement of the lower edge underneath the barrier.

In some non-limiting examples, block 1004 of the process 1000 can include coupling the instrument attachment to an instrument (e.g., a microscope). For example, this can include expanding the instrument attachment and retracting the expanded instrument attachment around the microscope (e.g., the objective of the microscope). In some cases, this can include coupling a lens to the instrument, in which case the lens can be coupled to the barrier. In other cases, this can include tightening a cuff around the instrument (e.g., the objective of the microscope).

At 1006, the process 1000 can include deploying the instruments, the sanitation systems, etc. In some cases, this can include advancing a surgical (or observation instrument) into the interior volume of the barrier so that a functional component of the surgical (or observation) instrument is situated entirely within the interior volume of the barrier. In some cases, the functional component can be implemented in various ways. For example, when a surgical instrument is used, the functional component can be a cutting edge, a drill bit, etc. As another example, when an observation instrument is used, the functional component can be an end of a tweezers, an point of probe, etc. In some non-limiting examples, this can include turning on a suction source to begin evacuating fluid (and thus other particulates) out of the interior volume of the barrier. In some configurations, this can include beginning causing the debris detection system to begin acquiring optical data of the interior volume of the barrier, and determining a current size distribution of particles within the interior volume of the barrier. In some cases, a computing device can determine the current size distribution of particles and determine whether or not the current size distribution of particles exceeds a threshold (e.g., a threshold size and a corresponding density of particles). If the computing device determines that the density of particles exceeds a threshold (and in some cases that the density of particles for a given size of a particles also exceeds a threshold), the computing device can cause the suction source to begin providing a suction source, or to increase the flow rate of the suction source.

At 1008, the process 1000 can include conducting a procedure, or an observation. In some cases, this can include conducting a surgical procedure, which can include cutting a portion of the patient and drilling a portion of the patient when the respective surgical instrument is positioned within the interior volume of the barrier. In other cases, this can include conducting an observation, which can include probing the sample, measuring features of the sample, etc.

At 1010, the process 1000 can include activating the disposal system of the isolation system. In some cases, after the procedure or observation is completed, but prior to activating the disposal system the technician or surgeon can cease operation of the instruments (e.g., a drill), an in particular, can cease the aerosol generating procedure. After ceasing the operation of the instruments, the technician or surgeon can wait a period of time (e.g., greater than one minute, two minutes, three minutes, etc.). In some cases, this can include maintaining that the instruments and arms of the personnel are maintained within the interior volume of the barrier. In this way, as the suction source continues to evacuate air out of the interior volume of the barrier, the remaining particulates and aerosols are evacuated.

In some cases, the instruments, and arms of the personnel can be removed (e.g., after the period of time has elapsed). For example, each arm can be retreated out of the respective arm port (and sleeve, if applicable), and each instrument can be retreated out of the respective instrument port. In some cases, block 1010 of the process can also include sealing each port. For example, each arm port can be resealed by engaging a plug with the arm port. In addition, each of the other ports can be resealed by engaging a plug with the respective port. In some cases, the plug can simply be a piece of material (e.g., fabric) that is coupled to the barrier to cover the port. In some configurations, only after the ports have been sealed, does the suction source turn off (e.g., after continuing evacuating material after a time period in which all the ports have been sealed). In this way, particulates and aerosols continue to be evacuated out of the interior volume of the barrier.

In some non-limiting examples, the disposal system can seal off particular portions of the barrier. For example, the disposal system can include one or more closing devices. Each closing device can decrease the cross-sectional area of the barrier to close off particular portions of the barrier. For example, a closure device can be situated below the instrument attachment and can be used to close off the interior volume of the barrier from the instrument attachment. Similarly, a closure device can be situated above the lower edge of the barrier and can be used to close off the interior volume of the barrier from the patient. In this way, with both closure devices closed, the remaining particulate and aerosols are trapped in the remaining interior volume between the closure devices. For example, the remaining interior volume of the barrier that is situated between the two closure devices can be removed from the site and disposed of, as appropriate. In some cases, closing the closure device can include tying an end of the barrier (e.g., with a tie), pulling a cable tie, etc.

At 1012, the process can include decoupling a portion of the barrier from the barrier. In some cases, this can include decoupling a portion of the barrier from the barrier along the area of material weakness (e.g., the perforation). In some non-limiting examples, this can include removing one closure device (and the remaining interior volume) form a portion of the barrier (e.g., the instrument attachment), and removing the other closure device from a portion of the barrier. In this way, the remaining interior volume of the barrier that is situated between the two closure devices can be removed from the site and disposed of, as appropriate. In some cases, closing the closure device can include tying an end of the barrier (e.g., with a tie), pulling a cable tie, etc.

At 1014, the process can include disposing the decoupled barrier. In some cases, the particulates and aerosols are trapped within the remaining interior volume of the barrier by using one or more closing devices, and thus the remaining interior volume of the barrier can be disposed of (or can be treated before being disposed of, such as using an autoclave system).

### EXAMPLES

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

### Example 1

COVID-19 has caused a global pandemic with a dramatic impact on healthcare systems. Concern for viral transmission necessitates the investigation of otologic procedures that use high-speed drilling instruments, including mastoidectomies, which were hypothesized to be an aerosol generating procedure. A Mastoidectomy was simulated using a high-speed drill and fresh-frozen cadaveric heads with fluorescein solution injected into the mastoid air cells. Specimens were drilled for one minute durations, in test conditions with and without a microscope. A barrier drape (e.g., the OtoTent) was fashioned from a commercially available drape. Dispersed particulate matter was quantified in segments of an octagonal test grid measuring 60 cm in radius. Drilling without a microscope dispersed fluorescent particles 360 degrees, with the areas of highest density in quadrants near the surgeon and close to the surgical site. Using a microscope or varying irrigation rates did not significantly reduce particle density or percent surface area with particulate. Using the OtoTent significantly reduced particle density and percent surface area with particulate across the segments of the test grid beyond 30 cm (which marked the boundary of the OtoTent) compared with the microscope only and no microscope test conditions (Kruskall-Wallis test, p=0.0066). Mastoidectomy with a high-speed drill is an aerosol generating procedure, a designation that connotes the potential high risk of viral transmission and need for higher levels of personal protective equipment. The barrier significantly reduced articulate dispersion in this study and could be an effective mitigation strategy in addition to appropriate personal protective equipment.

The novel severe acute respiratory syndrome coronavirus 2 (SARS-CoV-19) causing the disease COVID-19 emerged in Wuhan, China in November 2019 and has since spread rapidly across the globe, causing the World Health Organization to declare the outbreak a pandemic on Mar. 11 2020. Hospital systems in affected regions continue to face a surge of patients and struggle in the setting of shortages of testing materials, rapid testing strategies, ventilators, and personal protective equipment (“PPE”) for hospital staff.

Early reports of healthcare worker infections in China and Italy suggested high rates of infection among otolaryngologists. COVID-19 is believed to be spread through not only droplets but also aerosols during a variety of aerosol generating procedures that otolaryngologists routinely perform. These range from office-based procedures like flexible nasopharyngoscopy and peritonsillar abscess drainage to operative procedures such as intubation/extubation, tracheotomy, maxillofacial trauma surgery, as well as endoscopic sinus and shill base surgery.

Otologic surgery including mastoidectomy has not been explicitly described as an aerosol generating procedure (“AGP”), which is an important distinction that connotes the potential increased risk of viral transmission and the need for PPE designated for AGPs. However, existing studies suggest that the use of high powered drills is associated with the generation of aerosols and small particles with the potential to transmit infectious diseases. As the airway is continuous with the middle ear and mastoid, there is potential for viral transmission of COVID-19 from otologic procedures. An improved understanding of procedures that generate aerosols and small droplets is necessary to balance the need to protect health care workers with the desire to conserve limited stocks of PPE. In this study, we sought to demonstrate that mastoidectomy is an aerosol generating procedure and explored a barrier strategy to mitigate the risk for viral transmission.

Three ears from two fresh-frozen cadaveric head specimens were prepared. Standard C-shaped postauricular skin incisions were made and anteriorly based periosteal flaps were elevated. The Fluorescein solution was created with 50 mL of sterile water mixed with 1 mg of FUL-GLO Fluorescein Sodium (Akorn, Inc., Lake Forest, Ill., USA). The mastoid cortex was drilled to expose a 4×4 mm area of air cells and 1.5 mL of fluorescein solution was injected into this well. The Midas Rex© Legend Stylus otologic drill with a compatible Xomedit) 6 mm round fluted bur (Medtronic, Inc., Minneapolis, Minn., USA) was used. Images and videos were captured on a dual-lens camera system with a 12 MP camera with a wide-angle lens with f/1.8 aperture and 4K video recording system at 60 frames per second (Apple, Inc, Cupertino, Calif., USA). The microscope was a wall-mounted Zeiss OPMI Pico (Carl Zeiss Meditec AG, Jena, Germany) with an objective lens focal distance of 250 mm. An ultraviolet light source, UV-705, 400-Watt (Altman Lighting, Yonkers, N.Y., USA) was used and all fluorescent images were taken in a darkened room. A 1060 Steri-Drape of 130 cm×130 cm in size with an incise film in the center of 10 cm×12.5 cm was used (3M©), Inc, St Paul, Minn., USA) for the barrier drape.

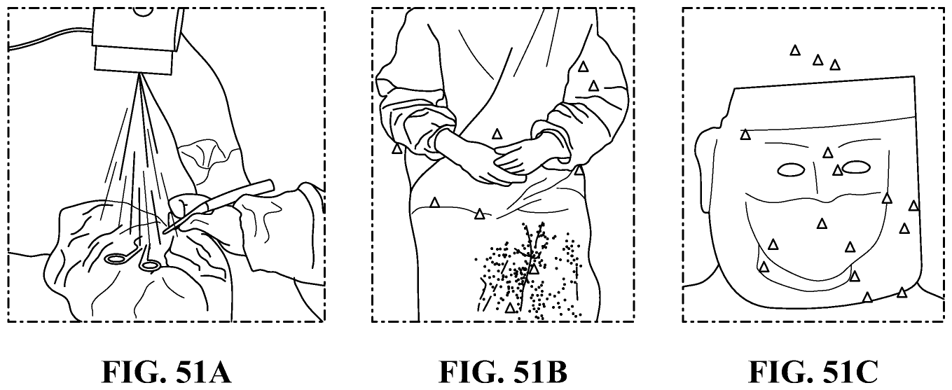

A separate cadaveric temporal bone was used for each primary condition. A single, right-handed surgeon drilled for one minute for each condition. The surgeons surgical gown and mask were photographed under ultraviolet light to evaluate for fluorescent debris (see, FIGS. 51A-51C). FIG. 51A shows the aerosolization of fluorescent bone dust and droplets occurring during a mastoidectomy. In particular, FIG. 51A shows that the aerosol plume created by using a high speed otologic drill to perform a cortical mastoidectomy is visible in a darkened room under ultraviolet light. The surgeon was using a size 6 cutting bur at 70,000 RPM, on a cadaveric specimen. FIG. 51B shows fluorescent debris (some indicated by arrowheads) soiling a surgeon's chest, arms, and lap under ultraviolet light after drilling part of a cortical mastoidectomy for 2 minutes, with size 6 cutting bur, at 70,000 RPM. FIG. 51C shows an image illustrating fluorescent particulate matter scattered on the surgeon's face shield and hair covering (shown with arrowheads) after 2 minutes of drilling.

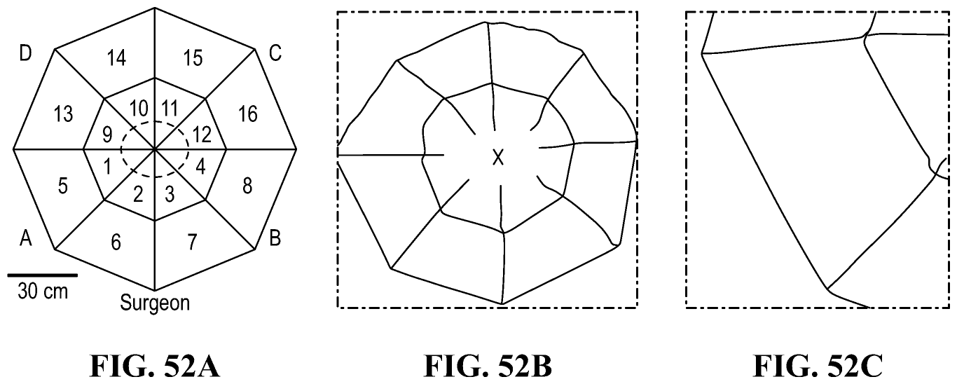

The cadaveric head was placed in the center of a black mat in a standard surgical position. Using the external auditor canal (“EAC”) as the center point, an octagonal grid with a radius of 60 cm was marked out around the specimen (see FIGS. 52A-52C). After each experiment, particulate matter was examined in each segment of the octagonal test grid (see FIG. 52A). The primary conditions tested were 1) open field (no microscope or barrier drape); 2) microscope without a drape; and 3) microscope with a drape. All three primary conditions were tested with 10 mL/min irrigation. Two additional conditions in an open field were tested: high irrigation (20 mL/min) and no irrigation. After drilling in each condition, photos were taken of the octagonal grid (see FIGS. 52A-52C) for further image processing and particle counts. The black mat was cleaned between experiments.

FIGS. 52A-52C show the experimental setup. FIG. 52A shows an octagonal grid to define distances and locations of particulate debris from the ear canal. The inner octagon has a radius of 30 cm and the outer octagon has a radius of 60 cm. The cadaveric head specimen was placed in the center (dotted circle). Segments of the grid are numbered (small font), and quadrants are labeled (large font) for reference in the text. FIG. 52B shows a sample of an aerial photo of grid under ultraviolet light with the cadaveric specimen marked by the “X”. FIG. 52C shows a sample of a close up photo of one segment of the grid with numerous fine fluorescent particles, representing bone dust or fluorescein stained droplets.

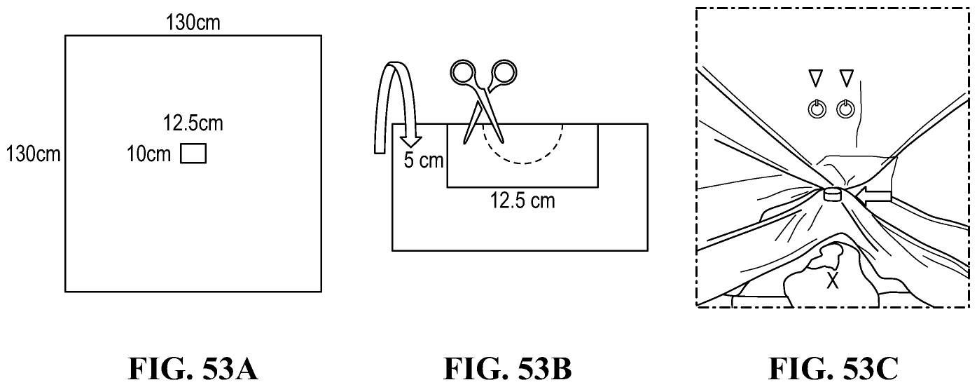

FIG. 53A shows the 1060 Steri-drape that was used to create a barrier drape that enclosed the microscope lens, cadaveric head specimen, and immediate surrounding 30 cm surgical field (here forward referred to as the “OtoTent”). A hole with a 6 cm diameter was cut into the incise film (with adhesive backing) in the center of the 1060 Steri-drape, which is shown in FIG. 53B. The hole in the drape was aligned with the microscope lens so that the lens was not obstructed, and the surrounding adhesive part of the drape was seemed around the outside of the lens mount (see FIG. 53C).

FIG. 53A shows the preparation of the OtoTent. In particular, FIG. 53A shows a sketch of an opened 1060 3M drape. The central 10×12.5 cm portion of the drape is backed in adhesive. FIG. 53B shows a sketch of how a hole is cut in the adhesive portion of the drape to allow for the microscope lens. FIG. 53C shows a photo of the OtoTent in position on the microscope, with the edge of the drape lifted. The microscope oculars (arrowheads), microscope lens (arrow) and cadaveric specimen (X) are marked for orientation.

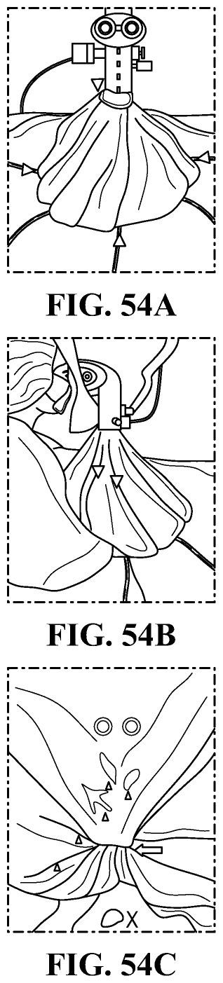

The drape was placed over the cadaveric head. The excess drape was loosely rolled wider and secured to the mat in four cardinal points 30 cm away from the EAC with tape: superior, inferior, posterior, and anterior (see FIG. 54A). The surgeon's arms and instruments were passed under the drape, on either side of the posterior point of tape fixation, to perform the surgery (see FIG. 54B). Following the experiment, the drape was lifted to reveal fluorescent tissue particles on the undersurface (see FIG. 54C).

FIG. 54A shows the positioning and use of the OtoTent. FIG. 54A shows that the drape was secured at cardinal points 30 cm away from the EAC of the specimen using adhesive tape: superior, inferior, posterior, and anterior (arrowheads). FIG. 54B shows an image of surgeon operating under the OtoTent. The black arrowheads indicate the positions of the hands underneath the drape. FIG. 54C shows an image of the underside of the OtoTent after drilling for 60 seconds. The edge of the drape is lifted up to show the fluorescent particles densely adherent to the underside of the drape (green arrowheads). The microscope lens (arrow) and cadaveric specimen (X) are marked for orientation.

ImageJ software (version 2.0.0-rc-69/1.52p) was used for image manipulation and particle analysis. Images were cropped to include only the segment to be analyzed in each iteration. The background was subtracted algorithmically using a rolling ball radius of 5 pixels, with separated colors and the sliding paraboloid method to remove background reflected light and heterogeneous surface reflection. Following this, color adjustment was performed to eliminate red and blue hues. The image was checked for consistency and non-particulate edges were removed manually. The image was changed to an 8-bit image, and a binary black and white pixel threshold was applied. Particle analysis was run with a particle size 0-infinity, circularity 0.0-1.0, with particle counting and percent area calculation. GraphPad Prism version 8.0 (La Jolla, Calif.) was used for descriptive statistics. Nonparametric tests (Kmskall-Wallis test, Mann-Whitney U test, two-stage linear step up procedure of Benjamini, Krieger, Yukutieli for multiple comparisons) were used to compare particle surface density and percent surface area covered by particles in different regions of the test grid.

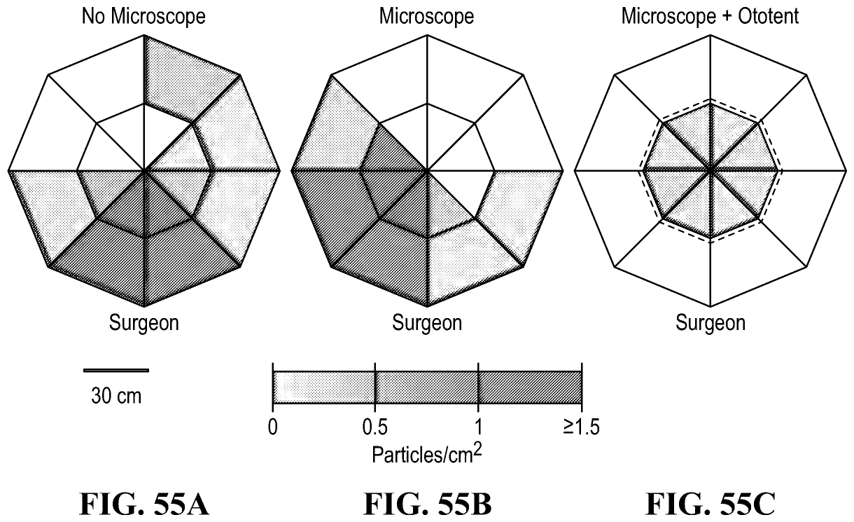

Videos and still images of drilling in an open field and drilling with a microscope demonstrated large plumes of fluorescent aerosolized materials (see FIGS. 55A-55C). Photographs of the surgeon's gown, face shield, and hair covering revealed a heavy burden of contamination within minutes of drilling (see FIGS. 55A-55C). The patterns of aerosol and particulate dispersion among the three test conditions (open field, microscope without OtoTent, and microscope with OtoTent) over the octagonal test grid are shown in FIGS. 55A-55C. Particles, including bone dust and fluorescein droplets, were found in every quadrant in every experimental condition. Particulate size ranged from 100 um to 4.6 mm, with >99%, of the particles being between 100 um and 1 mm in size. Due to the image resolution, particulates smaller than 100 um could not be evaluated.

FIGS. 55A-55C show a heat maps of the surface density of fluorescent particles found in each grid segment after each test condition. FIG. 55A shows a simulation without the microscope where there is a predominance of particles in the quadrants closest to the surgeon. There is also particulate dispersion away from the surgeon, illustrating the importance of considering strategies that offer protection to nearby operating room staff. FIG. 55B shows that the addition of the microscope still results in particulate dispersion that is highest in quadrants 1 and 2 (adjacent to the surgeon), and also still demonstrates particulate dispersion away from the surgeon (potentially toward other operating room staff). FIG. 55C shows a simulation with the OtoTent (dotted lines) where there is decreased particulate matter in all areas, including both the inner and outer octagons. Note the OtoTent drape was fixed at four cardinal points at a radius of 30 cm, thus enclosing the inner octagon on the grid. Note that particulate surface density is close to zero in the surrounding outer octagon, outside the OtoTent barrier.

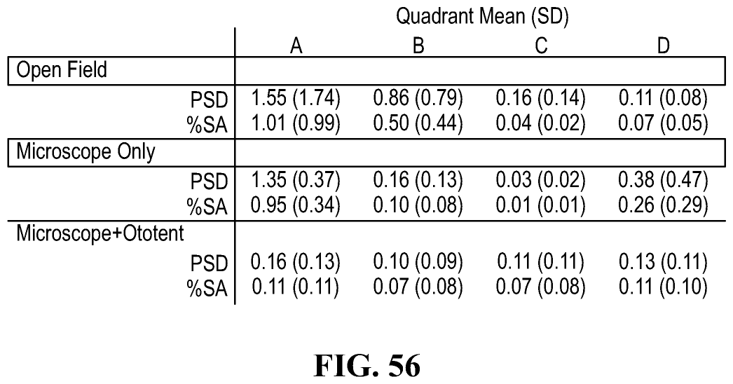

FIG. 56 shows a table for percent surface density (“PSD”) and percent surface area % SA) covered in particulate after drilling in each test condition. SD=standard deviation.

Fluorescein droplets and bone dust dispersed to all segments of the grid in a 360 degree fashion, with particle surface density (PSD) ranging from 0.036 to 4.0 particles/cm2 and percent surface area with particulate (% SA) ranging from 0.011 to 2.3%) (see FIG. 56). The highest PSD and % SA were found in quadrants A and B, representing quadrants closer to the surgeon (PSD 1.55±standard deviation, SD, 1.74, % SA 1.01±0.99; PSD 0.86±0.79, % SA 0.50±0.44 respectively). There was less particulate matter in quadrant C than the quadrants nearer to the surgeon (PSD OJ6:Hl14, % SA 0.04±0.02) and the least amount was found in quadrant D (PSD 0.11±0.08, % SA 0.07±0.05). Qualitative assessment of the table beyond the 60 cm radius of the octagonal grid revealed fluorescent particulate debris in both the front left and right corners of the table after drilling, at a distance of 114 cm from the EAC. Further distances could not be assessed with this experimental set up. Fluorescein droplets and bone dust were found in all areas of the experimental grid with PSD ranging from 0.011 to 1.74 particles/cm2 and % SA ranging from 0.004 to 1.3%. The highest PSD and % SA were found in quadrant A (PSD 1.35±0.37, % SA 0.94±0.34) and the lowest were found in quadrant C (PSD 0.03±0.02, % SA 0.01±0.01).

Fluorescein droplets and bone dust were found at low levels across all areas of the experimental grid with no areas of predominance in terms of the radial direction. PSD ranged from 0.018 to 0.29 particles/cm2 and % SA ranged from 0.008 to 0.25%. For the microscope+OtoTent condition, both PSD and % area of particulate were significantly lower in the outer circle (segments 5-8 and 13-16) (0.034+/−0.017, 0.020+/−0.010) compared to the inner circle (segments 1-4 and 9-12) (0.21+/−0.054, 0.16+/−0.065) (p<0.0001, Mann-Whitney U test). There was a large amount of fluorescent debris attached to the undersurface of the OtoTent, which may account for the apparent reduced levels of particulate debris even in the inner circle compared to other test conditions (though this did not reach statistical significance as noted below).

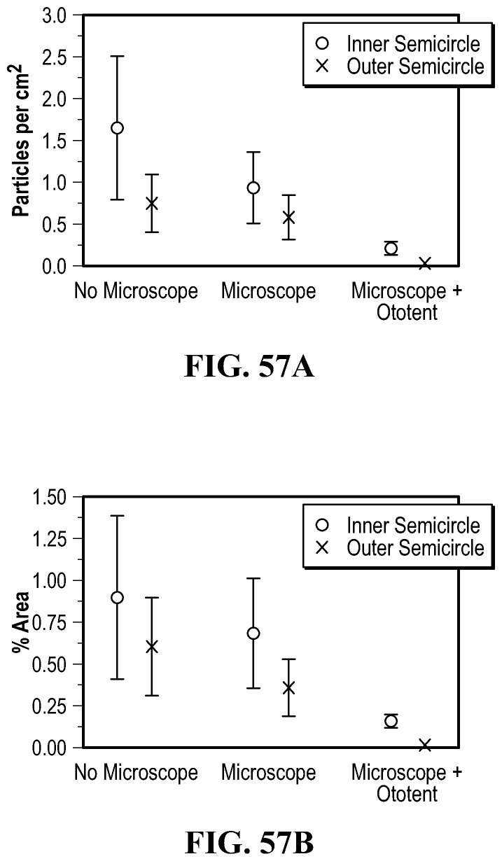

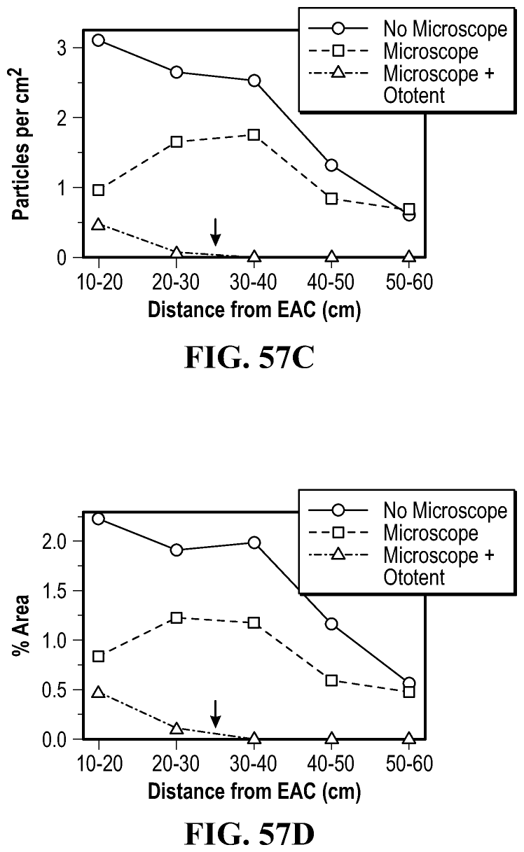

The Oto Tent quantitatively reduced particle dispersion beyond the boundaries of the drape. Quantitative comparisons across simulation conditions were performed by grouping the segments of the octagonal grid into inner circles (segments 1-4 and 9-12) and outer circles (segments 5-8 and 13-Hi). Since the majority of the aerosolized particulates were found in quadrants A and B of the grid, an analysis of these two quadrants was performed to compare dispersion between the inner and outer areas across test conditions. In this analysis, particles found in the inner semicircle (segments 1-4) were compared with those of the outer semicircle (segments 5-8) closest to the surgeon. Particle dispersion in terms of PSD and % SA are shown in FIGS. 57A, 57B, respectively. In the inner semicircle, comparisons of PSD and % SA were not statistically significantly different across the three test conditions (KruskaU-\Vallis test, p=0.074 and p=0.39, respectively). In the outer semicircle, comparisons of PSD and % SA were statistically significantly different across the three test conditions (Kruskall-Wallis test, p=0.0066). There was a statistically significant difference in both PSD and % SA in the outer semicircle between drilling without a microscope and drilling with the microscope+OtoTent (two-stage linear step up procedure for multiple comparisons, p<0.05 for particle density and for percent surface area). Similarly, there was statistically significant difference in both PSD and % SA between drilling with a microscope and drilling with the microscope+OtoTent (two-stage linear step up procedure for multiple comparisons, p<0.01 for PSD and % SA).

A subanalysis of segments 2 and 4, which had a high surface density of particulate matter was performed by further subdividing the segments into trapezoidal segments with a 10 cm radius as measured from the center to the perimeter of the octagonal grid. The central 10 cm triangular segment was not counted because this area was covered by the cadaveric head specimen. For the no microscope and microscope conditions, both PSD (see FIG. 57C) and % SA (see FIG. 57D) were highest between 10 to 40 cm and began to decrease at distances beyond 40 cm from the EAC. Note that there is still significant particulate measured at 60 cm from the EAC in both of these conditions. For the microscope+OtoTent condition, PSD and % SA were low inside the OtoTent and approached zero at distances greater than 40 cm from the EAC, representing the area outside the OtoTent.

FIGS. 57A-57D show graphs that quantify fluorescent particles under three test conditions: no microscope, microscope, and microscope+OtoTent in terms of particle surface density (FIG. 57A) and percent (%) surface area covered by particles (FIG. 57B). The mean of the inner semicircle (segments 1-4) and outer semicircle (segments 5-8) is shown, with standard error bars. There was no significant difference in either particle surface density or surface area covered when the inner semicircle segments were compared. The OtoTent condition showed a significantly decreased particle surface density and % area covered when the outer semicircle was compared to the no microscope and microscope conditions. Particulate dispersion as a function of distance from the EAC (FIGS. 57C, D) is shown based on a sub analysis of a single triangular wedge of the octagonal grid (segments 2 and 4). For both microscope and no microscope conditions, the particulate surface density and % area covered began to decrease beyond 40 cm from the EAC but were still present at 60 cm from the EAC. In the OtoTent condition, particulate density and % area approach zero beyond the OtoTent were measured, which was fixed at a 30 cm radius. Down-pointing at-row denotes the location of the perimeter of the OtoTent at 30 cm away from the EAC.

High-flow and low-flow irrigation conditions did not significantly change particle dispersion. Conditions with high- and low-flow irrigation did not significantly impact the patterns of aerosol and particulate dispersion. In comparing non-microscope drilling conditions with different irrigation parameters (low-flow irrigation at 10 mL/min, high-How irrigation at 20 mL/min, and no irrigation), PSD and % SA following drilling did not differ significantly between irrigation conditions for the inner semicircle (Kmskall-Wallis test, p=0.86, p=0.71, respectively) or outer semicircle of segments in the test grid (Kruskal-Wallis test, p=0.63, p=0.65, respectively).

Otolaryngologists are uniquely susceptible to COVID-19 transmission due to the variety of procedures performed on areas contiguous with the upper respiratory tract where there is a viral load. During this pandemic, otolaryngologists may be required to perform common sr-genies for m.gent indications and should prepare strategies to mitigate risk. These include pre-operative COVID-19 testing, 4 if timing and resources allow, as well as procedure specific strategies to decrease the risk of transmission from patients who are at risk or positive for COVID-19. In this paper, we examined the risks of contamination with biomaterials during mastoidectomy and introduce a novel risk mitigation strategy using a modified operating room drape.

As a cortical mastoidectomy is the treatment for a number of serious complications of acute and chronic otitis media, this aimed to characterize the spread of aerosolized materials during surgery that could potentially transmit virus. The use of high-powered drills has previously been demonstrated to generate aerosol-sized particles. In this study, it was demonstrated that mastoidectomy is an aerosol generating procedure with the ability to spread small droplets more than 100 cm from the surgical site, with a predominance of spread in the areas closest to the operative site. The experimental set up was designed in a 360-degree fashion to assess risk of aerosolized debris dispersion toward all operating room staff in close proximity, including the anesthesiologist and the scrub nurse or technician. While the majority of the particulate debris was found the in the two quadrants adjacent to the surgeon, this study also demonstrates that cortical mastoidectomy may cause particulate spread in the two quadrants located opposite the surgeon. This highlights the importance of barrier drapes hung between the surgical site and the anesthesiologist. Furthermore, in this study, the right-handed surgeon spread aerosolized debris predominantly in the left lower quadrant of the field, followed by the right lower quadrant. This may have been impacted by accumulation of some particulate on the surgeon's arms and gown, reducing the measured particulate in the lower right quadrant. Aerosolized particles can be found all over the surgeon including on the gown, face shield and hair covering. These findings corroborate prior studies examining the possibility of transmission of blood borne and prion diseases during mastoidectomy, finding that drilling scatters blood-containing and neural tissue-containing material that could be detected on the surgical field and on the surgeon.

Although it is not known for certain whether aerosols generated during mastoidectomy are capable of transmitting COVID-19, existing virology literature suggests that fluid in the inner ear and mastoid can be infected with respiratory viruses. Previous studies demonstrated that viral RNA could be identified in 48% of middle ear fluid samples collected from children with an upper respiratory illness and acute otitis media when assessing for human coronavirus, respiratory syncytial virus and human rhinovirus, Similarly, another study found that viral materials could be identified with enzyme immunoassays in 74% of middle ear fluid samples in children with acute otitis media when assessing for parainfluenza, influenza, respiratory syncytial virus, enterovirus and adenovirus.

While there are no formal guidelines on the best practices to reduce viral transmission during common otology or neurotology procedures, novel techniques have recently been reported to mitigate the risk associated with oral intubation, extubation, and endoscopic sinus surgery. These strategies make use of various plastic materials to create physical barriers between the patient and the health care provider. In this study, as the microscope alone was shown to be an insufficient barrier, a barrier drape was used (the OtoTent) to limit the spread of aerosols and droplets during mastoidectomy. The drape was created and affix to the microscope. It was found that fixing the tent posteriorly with tape between the surgeon's arms was critical to keeping the OtoTent in place during surgery. The surgeon's arms and instrument cords were easily passed between points of fixation with good range of motion. The OtoTent significantly reduced droplet and particulate contamination of surfaces beyond its borders within the limits of this study design. In addition, particulate debris inside the borders of the OtoTent on the surfaces of the cadaver's head and immediate surrounding area was also significantly reduced. This is likely due to particulate debris adhering to the undersurface of the OtoTent.

The OtoTent was created from a commercially available surgical drape commonly used for ophthalmologic procedures. Hospitals with ophthalmology divisions may already carry the 1060 drape. The cost of the product is low, around $10 US dollars. A microscope drape could be used as an alternative material but may be more expensive and may require more manipulation to affix it securely to the microscope. Various improvements can be made to the simple OtoTent design presented in this study—the authors elected to tape from cardinal points on the drape to minimize movements to the drape with the surgeon's hand movements. During drilling, the surgeon may find that the microscope lens needs to be cleaned as debris and moisture circulates under the OtoTent so a wipe should be kept within easy reach. A surgical scrub technician could pass additional instruments underneath the other flaps of the OtoTent not occupied by the surgeon's two arms. At the conclusion of drilling, the OtoTent should be removed carefully so as not to dislodge and re-aerosolize particles. It may be beneficial to wait a short time to allow for settling of at least the larger aerosolized particles. A second set of surgical gloves and arm sleeves could be used under the OtoTent and removed at the conclusion of drilling to minimize dispersion of particles landing on the surgeon. Because the OtoTent is not impervious around its perimeter, it is not a substitute for appropriate personal protective equipment (“PPE”) and should only be used as an adjunct.

Even with success of a material barrier like the OtoTent, mastoidectomy may also produce microscopic aerosols that could remain airborne for an extended period of time. In this study, the visualization of plumes of fluorescent debris during mastoidectomy demonstrate that it is certainly an aerosol generating procedure despite the limitations of this study to analyze particles smaller than 100 μm. Previous studies conducted a cadaveric temporal bone study to sample aerosolized bone dust in the air during mastoidectomy and a found that the average total particulate matter concentration was 1.89 mg/m³. Although it was concluded that this was below the Occupational Health and Safety Administration's standards for respirator use, it was not explored that the idea that a small amount of particulate matter might be enough to transmit a virus.

What is yet unknown is the ability of the aerosolized materials produced during mastoidectomy (e.g. blood, bone dust, middle ear and mastoid mucosa and fluid) to transmit COVID-19 and whether the quantity and size of particles affects the transmission rate. As such, this study reinforces the recommendations in the existing literature to remain vigilant in the selection of appropriate PPE. At a minimum, for mastoidectomy surgery, basic or attire with impervious gowns, gloves, hair coverings, and shoe coverings should be supplemented with face shields, ventless or wrap-around eye protection and respirators. N95 respirators should be used as for all aerosol generating procedures as recommended by multiple medical professional societies and the World Health Organization. As mastoidectomy is an aerosol generating procedure, it is believed that N95 masks or more advanced levels of protection are warranted. Powered air purifying respirators (“PAPRs”) have been recommended in the field of orthopedic surgery to decrease the biomaterials that touch the surgeon during bone drilling procedures. Given that a high degree of spread of aerosolized debris was found in this study and that particulate debris was found even in the hair covering after 1 minute of drilling, a PAPR and an N95 mask for COVID-19 positive patients and patients with unknown COV1D-19 status is favored. This combination has been shown to have a multiplicative effect on reducing the concentration of airborne particles. However, with limited availability of PAPRs at most institutions, we recognize that obtaining PAPRs may not be possible and thus have listed the minimum recommended PPE for mastoidectomy above. The use of PAPRs with microscope oculars may also be cumbersome, although it was possible to use a face shield with the microscope in this study. Exoscopes may be an alternative option in institutions with access to this technology.

The limitations of this study stem from constraints on the materials available to conduct these time-sensitive experiments during a time of medical crisis. The bone of cadaveric models may differ from those of living patients in their biochemical properties and their reactions to drilling. Also no equipment was used to measure the smallest aerosols that remain suspended in the air; the smallest particle that could be detected using techniques described in this paper was 100 μm. Bone dust, which had significant autofluorescence, was unable to be distinguished from droplets containing fluorescein, although bone dust itself could be mixed with mastoid fluids during drilling and could also harbor viral particles. Furthermore, it was not possible to assess individual particles that may have conglomerated upon hitting experimental surfaces. Finally, alternate configurations of the operation were not tested including the use of different drill speeds, burr types/sizes, suction irrigators, other microscope sizes/configurations, other drilling techniques (including a left-handed surgeon), and the use of unconventional alternatives to high speed drills such as osteotomes or hand-operated perforators. Limiting testing conditions was necessary to conserve resources including PPE. Lastly, the OtoTent was designed to allow for easy reproduction. More elaborate designs including the incorporation of gloves into the drape to minimize the escape of even smaller aerosolized materials could also be considered.

The aerosolization of fluorescent droplets and bone particulate from cortical mastoidectomy was demonstrated on a cadaveric specimen under an ultraviolet light in a darkened room. A heat map was created that shows the surface density of fluorescent particles found in each grid segment after test conditions increasing or decreasing irrigation. The surgeon drilled for 1 minute with a 6 mm round fluted burr after injection of fluorescein. Irrigation was increased to 20 cc/min, and then irrigation was turned off.

### Example 2

Some non-limiting examples of the disclosure provide systems and methods for mitigating airborne aerosol dispersion during mastoidectomy and provide systems and methods for custom mitigation strategies for otologic surgery in the COVID-19 era.

During the acute phase of the COVID-19 pandemic, major disruptions occurred in the healthcare sector. The initial closure of clinics and cancellations of non-urgent operations significantly impacted otolaryngology practices. As the COVID-19 infection rate plateaus and begins to decline across the country, clinicians require strategies to safely re-open practices, particularly in the setting of persistent shortages of widely available testing, personal protective equipment (“PPE”), and a lack of contact tracing in the community as has been attempted in other countries.

Otologists and lateral skull base surgeons may be at increased risk for occupational exposure as studies show that the use of a high-powered drill is associated with aerosol generation. The Centers for Disease Control (“CDC”) and World Health Organization (“WHO”) have recommended higher levels of PPE for aerosol generating procedures. Local source control may be an effective adjunctive strategy to mitigate viral transmission risk; however, there are currently no standardized local source control strategies for otologic surgery. In a prior study, the plume of aerosolized debris generated by mastoidectomy was shown, droplet and particulate (≥100 μm) dispersion in a 360-degree field around the surgical site was quantified, and the effectiveness of a barrier drape attached to the microscope (OtoTent) for reducing large particulate and droplet dispersion was demonstrated. Herein, the generation of aerosols between 1 and 10 μm in size was investigated. Furthermore, the efficacy of two barrier strategies to decrease exposure to these intraoperatively generated aerosols, including the previously described OtoTent and a novel prototype customized for otologic surgery, the OtoShield was evaluated.

The protocol was deemed exempt by the Institutional Review Board. Surgical simulation was performed on six ears from three thawed, fresh-frozen cadaveric head specimens. All experiments were performed in a surgical laboratory set at 72° F. and equipped with air exchangers operating at a rate of six air changes in the room per hour. Specimens were prepared with a C-shaped postauricular skin incision. An anteriorly-based periosteal flap was elevated. A single, right-handed surgeon completed all surgical conditions. The surgeon performed a cortical mastoidectomy and drilled for one minute for each condition. The microscope was a wall-mounted Zeiss OPMI Pico (Carl Zeiss, Meditec AG, Jena, Germany) with an objective lens focal distance of 250 mm. The Midas Rex© Legend Stylus otologic drill with a compatible Xomed© 6 mm round fluted bur and 5 mm diamond bur (Medtronic, Inc., Minneapolis, Minn., USA) was used at 70,000 RPM for drilling. The otologic drill had an attached irrigation port set to 10 mL/min. A 12-French suction was used in the surgeon's non-dominant hand, with the suction tip maintained approximately 1 cm from the drill bur, in all conditions except the “no suction” and “suction irrigator” conditions. The 12-Fr suction connected to wall suction in the laboratory which applied 538 mmHg suction pressure, and resulted in 32 L/min air flow rate.

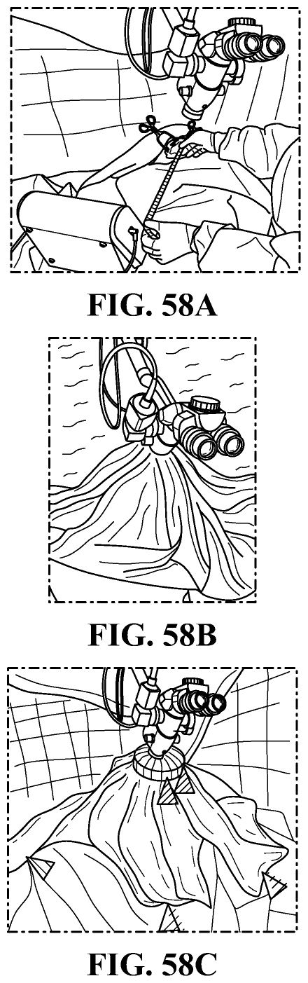

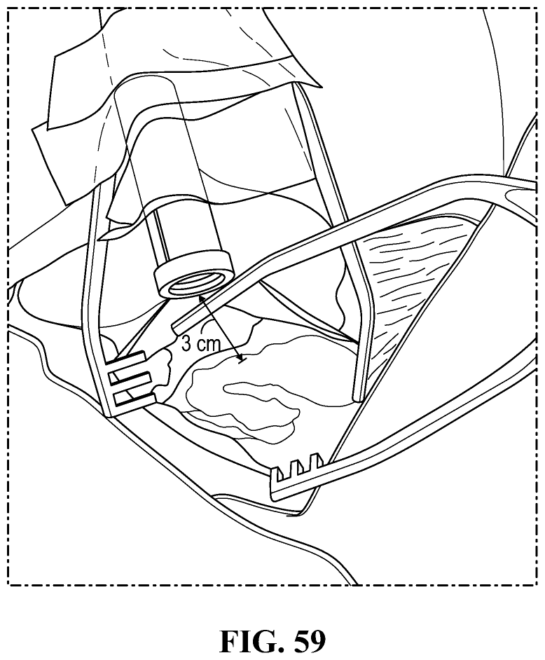

An optical particle sizer (OPS 3330, TSI Inc., Shoreview, Minn.) placed 30 cm from the ear canal (see FIG. 58A) measured particle number and size distribution. Single particle counting technology was used to measure particles 1-10 μm in size. The optical particle sizer had a flow rate of 1.0 L/min through a 3 mm port. Particle size distribution was measured in 16 channels. Total particle counts by size were collected in 10 second intervals for the duration of each experiment with replicates performed for each test condition. Background measurements were taken before each experiment for 60 seconds to ensure no change in the baseline aerosol concentration between experiments. Aerosol sampling was performed for the duration of mastoid drilling and continued until particle aerosolization returned to pre-experiment baseline.

Two types of barrier drapes were fashioned. The “OtoTent” was created with a 1060 Steri-drape that enclosed the microscope lens, cadaveric head specimen, and immediate surrounding 30 cm surgical field (see FIG. 58B) as previously described. A circle with a 6 cm diameter was cut into the incise film (which has an adhesive backing) to secure the drape to the outer perimeter of the microscope lens. The OtoTent was draped over the surgical field and secured in three cardinal locations. The surgeon's hands and instruments were passed under the drape to access the surgical field.

FIGS. 58A-58C show examples of various experimental setups. FIG. 58A shows a configuration that has no barrier during a drilling condition with the use of the Zeiss microscope. As shown in FIG. 58A, the optical particle sizer was placed 30 cm from the cadaveric ear canal. FIG. 58B utilizes an OtoTent barrier. FIG. 58C utilizes an OtoShield barrier with a built-in arm ports, enclosed floor, instrument/suction ports, and collapsible frame.

The “OtoShield” was a custom prototype drape design created from a Zeiss OPMI microscope drape (Carl Zeiss, Meditec AG, Jena, Germany; see FIG. 58C). It was attached to the outer perimeter of the microscope lens with a 9 cm opening and secured with an elastic cinch cord. The OtoShield contained two arm ports to accommodate the surgeon's hands, with reinforced stiffened entry points for easy arm placement. The arm ports were not sealed around the surgeon's arms. A third port accommodated the suction and otologic drill and was sealed circumferentially with a piece of Velcro. The OtoShield created a 3-dimensional enclosed space with a plastic drape that formed the “floor.” A 12 cm diameter hole was cut into the “floor” and adhered to the cadaveric head around the surgical site to allow the surgeon to access to the surgical field. Neither the OtoTent nor the OtoSheild was an entirely sealed system (e.g., there were potential sources of air leak. Volumes for the OtoTent and OtoShield were calculated based on a truncated cone shape and pyramidal shape, respectively.

Where indicated, a second suction (SS, Cardinal Health, 3/16″×6′, Dublin, Ohio, USA) was attached to the cadaveric head specimen 3 cm from the surgical site at the mastoid cortex to continuously evacuate ambient particles (see FIG. 59). The SS was connected to a second wall suction (separate from the one with the 12-Fr suction), with measured air flow rate of 65 L/min. FIG. 59 shows the experimental setup using a second suction. As shown, the suction tubing was attached to the cadaver 3 cm from the mastoid cortex to continuously evacuate particles.

A cortical mastoidectomy was performed under the microscope (with no barrier drape) under the following conditions: 1) use of 12 French suction in the surgeon's non-dominant hand; 2) use of 12 French suction and a SS attached at a fixed position with continuous evacuation of particles; 3) no suction use; 4) use of a 10/12 French suction irrigator; 5) use of a 5 mm diamond bur with a 12 French suction. Unless noted, all mastoidectomy procedures were performed with a 6 mm round fluted (“cutting”) otologic bur.

To assess the two barrier drapes, the following conditions were tested with simulated cortical mastoidectomy: 1) no barrier drape; 2) OtoTent; 3) OtoShield (FIG. 59C). Each condition was tested with and without the use of a SS fixed in the surgical field to continuously evacuate particles. The SS was turned on at the start of drilling and left on during barrier removal and subsequent particulate measurements. In both barrier conditions, the drape was removed either immediately upon cessation of drilling or after a 60 second rest period. The surgeon's arms were removed from the field at the conclusion of drilling regardless of whether the drape was removed in an immediate or delayed fashion.

Stata version 13 (StataCorp, College Station, Tex.) software was used for statistical analysis to assess differences in airborne aerosol generation above matched, specific pre-replicate baseline values for all test conditions. Non-parametric statistical techniques were utilized due to small sample sizes, with Bonferroni correction for multiple comparisons. Prism Version 8 (GraphPad Software, La Jolla, Calif., USA) was used to graph data. All values are reported as means with standard error.

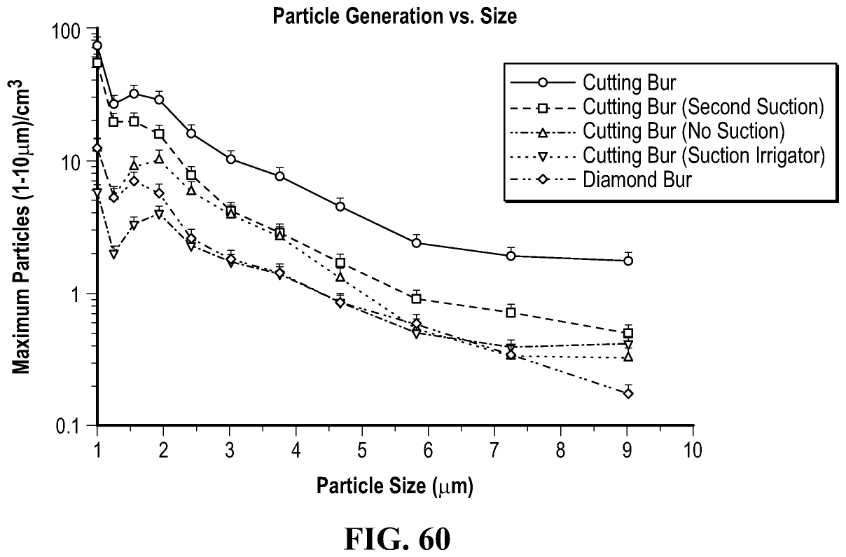

Small particle aerosols generated by drilling without a barrier drape for multiple conditions is shown in FIG. 60. The total number of particles detected 30 cm away from the surgical site was 409.9±150.2 particles/cm³ for the use of a (a) cutting bur, (b) 257.8±127.1 cutting bur with a SS, (c) 103.8±2.2 cutting bur with no suction, (d) 45.4±23.7 cutting bur with a suction irrigator, and (e) 76.4±70.1 diamond bur, respectively. All conditions followed a similar trend with smaller particles representing the largest proportion of total particles generated. Use of the cutting bur generated the greatest number of total particles, while slight modifications to the surgical technique decreased the average number of particles generated (Kruskall Wallis test, KW statistic 11.99, n-2-4 per group), with significant decreases seen with suction irrigator use (p<0.01) and diamond bur use (p<0.05, Dunn's multiple comparisons test).

FIG. 60 shows a graph of the maximum number of particles generated during 10 second increments with a flow rate of 1 L/min across particle size for mastoidectomy without a barrier in the following conditions: cutting bur, cutting bur with second suction, cutting bur with no suction, cutting bur with suction irrigator, and diamond bur. All conditions followed a similar trend with smaller particles representing the largest proportion of total particles generated. As shown, mastoidectomy with a cutting bur generated the greatest number of particles, with significant decreases in the suction irrigator use (p<0.01) and diamond use (p<0.05, Dunn's multiple comparisons test) conditions.

The average particle density across time is shown for mastoidectomy without a barrier drape in two drilling conditions: (1) cutting bur and (2) cutting bur with SS (see FIG. 60). The background level of particle detection was low prior to drilling in both conditions; 0.057±0.038 and 0.048±0.026 particles (1-10 μm)/cm³, respectively. No statistical difference was found between the two conditions for particle density generated over a 60 second drilling period. The peak particle density occurred in a delayed fashion in both conditions, with maximum particle density noted 30 to 40 seconds after drilling.

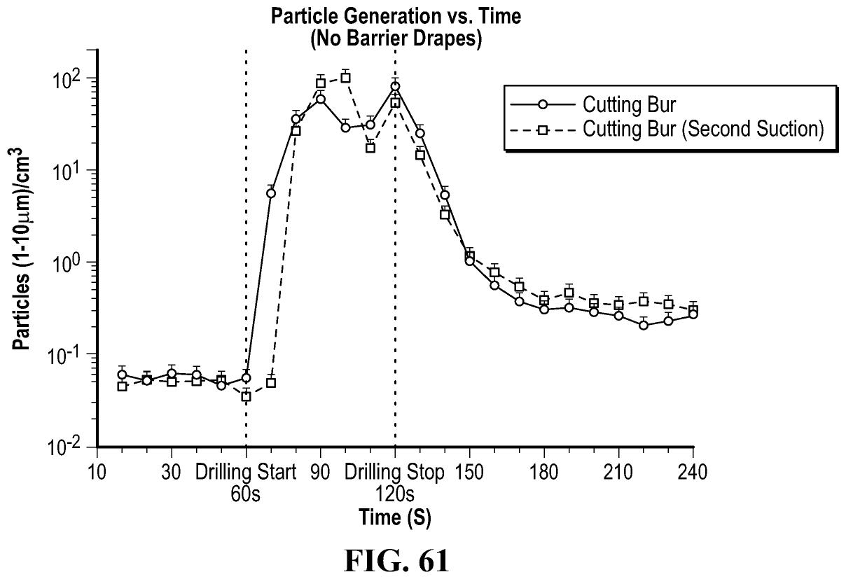

FIG. 61 shows a graph of the average particle density across time for mastoidectomy without a barrier in two conditions: cutting bur and cutting bur with a second suction. Drilling occurred between time points 60-120 seconds. No statistical difference was found between particle density compared to background levels in the two conditions.

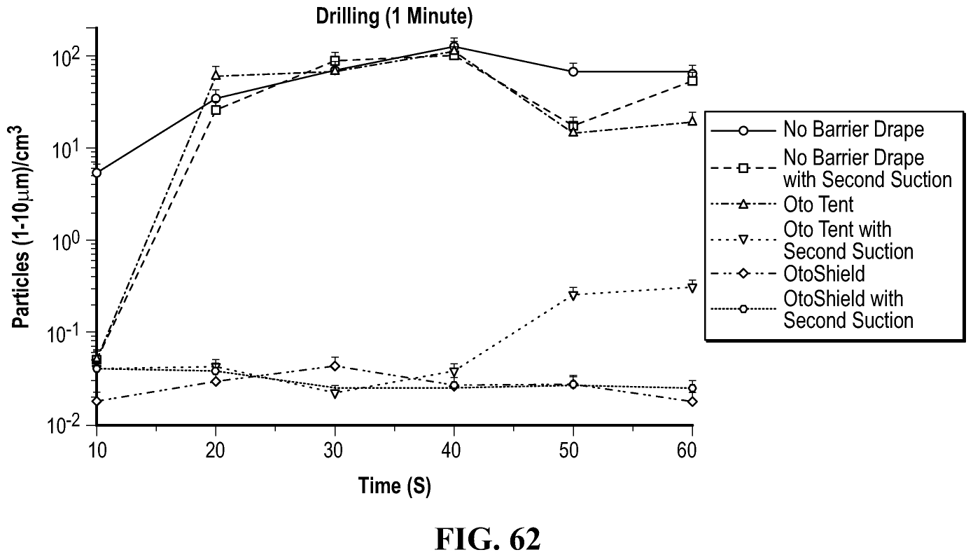

FIG. 62 shows a graph comparing the particle density generated in mastoidectomy without a barrier and with the OtoTent and OtoShield, across time, and with and without a second suction. Three of the conditions (mastoidectomy without barrier drape [p<0.001, U=57], mastoidectomy without barrier drape but with second suction [p<0.001, U=95], and OtoTent without second suction (p<0.001, U=107), showed high rates of particle generation during drilling compared to background levels of particle density (n=24 per condition, Mann-Whitney U Test, Bonferroni correction for multiple comparisons). The remaining conditions (OtoTent with second suction, OtoShield without second suction, and OtoShield with second suction) showed lower levels of particle generation during drilling, and the number of particles generated was not found to be statistically different from that in background levels for each of these conditions.

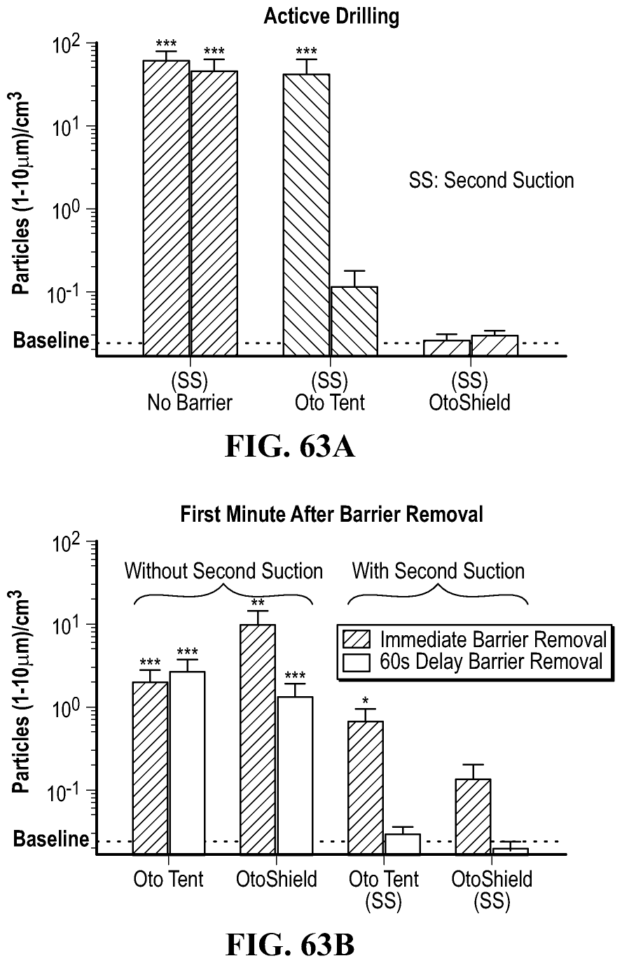

Comparison of particle density generated in the mastoidectomy without a barrier drape condition and the two barrier strategies, OtoTent and OtoShield, with and without the use of SS is shown in FIG. 62. Three of the conditions (mastoidectomy without barrier drape [p<0.001, U=57], mastoidectomy without barrier drape but with SS [p<0.001, U=95], and OtoTent without SS (p<0.001, U=107), showed high rates of particle generation during drilling compared to background levels of particle density (n=24 per condition, Mann-Whitney U Test, Bonferroni correction for multiple comparisons). The remaining conditions (OtoTent with SS, OtoShield without SS, and OtoShield with SS) showed lower levels of particle generation during drilling, and the number of particles generated was not found to be statistically different from that in background levels for each of these conditions (see FIG. 63A).

FIG. 63A shows a graph of the particle density generated during one minute of drilling for Mastoidectomy without a barrier drape, mastoidectomy without a barrier drape but with second suction, and OtoTent without second suction that showed high rates of particle generation during drilling. Conversely, OtoTent with second suction, OtoShield without second suction, and OtoShield with second suction showed low levels of particle generation during drilling.

The effect of delaying barrier removal by 60 seconds following completion of drilling is shown in FIG. 62B. Delaying barrier removal when using an OtoTent without SS still demonstrated significant aerosol dispersion (p<0.001, U=0, n=10,12). While delaying barrier removal when using an OtoShield without SS proved slightly beneficial compared to immediate removal, significant aerosol was still generated (p<0.001, U=2, n=12,12). However, delaying barrier removal when using an OtoTent with SS or OtoShield with SS mitigated aerosol dispersion to levels not significantly different from background.

FIG. 63B shows another graph of the particle density generated following barrier removal either immediately or after one minute had elapsed after drilling. Particle density generated after barrier removed immediately and after one minute for the conditions OtoTent with and without a second suction and OtoShield with and without a second suction. Delaying barrier removal when using an OtoTent without a second suction did not decrease particle dispersion. Delaying barrier removal when using an OtoTent or OtoShield with a second suction significantly decreased aerosol dispersion levels.

The COVID-19 virus may be transmissible through otologic and neurotologic surgery as the fluid and mucosa of the middle ear and mastoid are contiguous with that of the upper respiratory tract where the viral load is high. Other respiratory viruses, such as human coronavirus, rhinovirus, respiratory syncytial virus, influenza, parainfluenza, enterovirus and adenovirus, have been identified in middle ear fluid samples from children with upper respiratory illnesses. Although we are unaware of studies showing Sars-CoV-2 in the middle ear, it is prudent to assume a potential risk of otologic transmission. While Sars-CoV-2 is primarily spread via droplet transmission, it can act as an opportunistic airborne infection, particularly in the setting of aerosolizing procedures.

This study confirms the generation of aerosols between 1-10 μm in size during mastoidectomy, complementing existing research of larger particles generated during mastoidectomy procedures. Within the limits of comparison given differences in experimental techniques and conditions, mastoidectomy appears to generate more aerosol dispersion than intubation (not including cough or sneeze), and anterior skull base drilling. There is a paucity of experimental data for small particulate mastoidectomy aerosolization and our data could not be compared to a prior study with a gravitational spectrometer. Modifications to surgical technique, including use of a suction irrigator and diamond bur significantly decreased total particle dispersion. The reduction in particulate with a suction irrigator may be due to increased irrigation resulting in larger droplets that contain particulate rather than small airborne droplets and particulate. Notably, there was qualitatively more visible large droplet splatter and dispersion with the suction irrigator.

Two barrier strategies were investigated to mitigate aerosols produced during mastoidectomy. The OtoTent is created from a commercially available, low cost drape that can be attached to any microscope or exoscope. The design is described in a prior study, which demonstrated a statistically significant 360-degree reduction of larger aerosols and droplets produced during mastoidectomy with use of the OtoTent compared to a microscope alone. The OtoShield is a custom prototype drape with specialized ports to accommodate the surgeon's arms and instruments. This three-dimensional design was made from clear polyethylene-based plastic and forms an enclosed space over the surgical site, including a plastic floor that is contiguous with the sides. The floor prevents larger droplets, particles, and liquids from leaking, and decreases aerosol escape through the bottom edge of the drape. The drape can include a rigid frame to keep the operating space unobstructed by drape material. The customized arm ports allow the surgeon to access the surgical field. Surgeons who trialed the OtoShield in the laboratory noted that it was comfortable to use, and did not obstruct the view of the surgical site.

The OtoShield without SS significantly reduced aerosol dispersion during drilling trials compared to mastoidectomy without a barrier (with or without SS). The use of the OtoTent without SS did not significantly decrease aerosol dispersion, which may have been from inadvertent aerosol dispersion generated by lifting the drape to access the surgical field. Notably, however, we previously described that the OtoTent decreased dispersion of large aerosols and droplets (>100 μm). Use of the SS within the drape significantly reduced particle dispersion in both the OtoTent and OtoShield. Results of the OtoTent with SS were more variable than that of the OtoShield with SS. In 75% of trials, the OtoTent with SS significantly decreased particle dispersion throughout drilling; in 25% trials, there was notable aerosol in the last 30 seconds of drilling, though these results were not statistically significant.

These variable results may be attributed to inconsistencies in drape position between trials, suggesting that a customized drape (e.g., OtoShield) may minimize variability. Of note, however, even partial mitigation of aerosol dispersion is beneficial as the infectious potential of biomaterials is related to the viral load of exposure.

Placement of the SS within the drape is critical for decreasing particle dispersion, likely due to an increased volume of air turnover within the drape. The volume of the OtoTent and OtoShield barrier drapes were 40L and 37L, respectively. The flow rate of the SS was 65 L/min, such that the entire volume within the drape exchanged during drilling. In contrast, the flow rate of the 12-Fr suction was 32 L/min; thus, the volume within the drape was not replaced during drilling. Further, the 12-French suction was employed to remove fluid, rather than open to air to scavenge aerosols. Simultaneous application of multiple strategies including (1) use of the barrier drape, (2) delaying drape removal, and (3) increased air turn over via the SS were important.

Static methods for aerosol assessment, cadaveric models, and the natural variability in aerosol generation from high speed drilling were all variable factors. This study measured optical particle size without the use an aerodynamic particle sizer or dynamic assessment techniques, and did not account for change in droplet size, dessication, or formation of droplet nuclei over time. Particulate density was measured at only one location in the surgical field. Small droplets and bone dust particulate could not be distinguished. The presence of infectious pathogens, including virus or bacteria, in the aerosol were not assessed. Longer drilling times were not included given the limited cadaveric resources, and only mastoid cortical bone was drilled in this study in order to limit variance from differences in surgical site bone. It is possible that longer drilling times could result in higher aerosol density within the barrier drapes and thus more aerosol escape despite a second suction. Further research is needed to determine the optimal length of the rest period prior to drape removal and instrument exchange, as it will depend on duration of drilling, leakage rate of barrier design, and suction air flow rate. Drilling in cadaveric bones may not be analogous to drilling in living patients as the bones have different composition and lack viable mucosa and mucous. Future studies should include use of the barrier drape strategies on patients in the operative room setting and should include testing for droplets as well as aerosols. Alternative designs of the barrier shields could be investigated, as well as different drilling conditions including a range of drilling speeds, bur sizes and types, as well as different microscopes or exoscopes. Despite the success of the barrier strategies, personal protective equipment (“PPE”) should not be reduced as this study has not been replicated in a clinical setting.

Mastoidectomy using a high-speed drill is an aerosol generating procedure with the potential to spread infectious particles smaller than 10 μm. Use of a barrier drape is an effective strategy to mitigate dispersion of aerosols. A customized OtoShield minimizes variability in aerosolization and may be preferred over the OtoTent. Other strategies, such as use of the SS and delayed removal of the drape after drilling, should be used in conjunction with a barrier drape strategy to decrease particle dispersion. Even a partial decrease in aerosol dispersion, however, is beneficial as it may decrease the viral load of exposure. These highly successful strategies may be used as an adjunctive strategy to appropriate PPE during the COVID-19 era.

Some non-limiting examples of the disclosure investigate aerosols generated for mastoidectomy relevant to viral transmission during the COVID-19 era and explore source control mitigation strategies. In some non-limiting examples, an optical particle size spectrometer was used to quantify 1-10 μm size aerosols 30 cm from mastoid cortex drilling. Two barrier strategies were evaluated: (1) “OtoTent”—a drape affixed to the microscope; (2) “OtoShield”—a customized drape which enclosed the surgical field with specialized ports. As described, mastoid drilling without a barrier drape generated significantly higher aerosol density than background levels (p<0.001, U=57). Mean particle density measured with mastoid drilling under the OtoTent with SS, OtoShield without SS, and OtoShield with SS was not statistically different from that in background levels. By contrast, OtoTent without SS showed high rates of particle aerosolization during drilling compared to background levels (p<0.001, U=107). Delaying removal of the drape for one minute after drilling did not significantly decrease aerosols in the OtoTent or OtoShield without SS conditions (p<0.001, U=0, n=10, 12 and p<0.001, U=2, n=12, 12, respectively). However, delaying barrier removal when using an OtoShield or OtoTent with SS mitigated aerosol dispersion to levels not significantly different from background. As shown, mastoidectomy without a barrier generated significant aerosols. OtoTent with SS and OtoShield with SS, with delayed drape removal, were the most effective strategies. The customized OtoShield produced less variability compared to the simple OtoTent and may be considered to reduce the potential for aerosol dispersion.

Although some of the discussion above is framed in particular around systems, such as the various isolation system, those of skill in the art will recognize therein an inherent disclosure of corresponding methods of use (or operation) of the disclosed systems, and the methods of installing the disclosed systems. Correspondingly, some non-limiting examples of the disclosure can include methods of using, making, and installing isolation systems.

Although the invention has been described and illustrated in the foregoing illustrative non-limiting examples, it is understood that the present disclosure has been made only by way of example, and that numerous changes in the details of implementation of the invention can be made without departing from the spirit and scope of the invention, which is limited only by the claims that follow. Features of the disclosed non-limiting examples can be combined and rearranged in various ways.

Furthermore, the non-limiting examples of the disclosure provided herein are not limited in application to the details of construction and the arrangement of components set forth in the following description or illustrated in the following drawings. The invention is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Also, the use the phraseology and terminology used herein is for the purpose of description and should not be regarded as limiting. The use of “right”, “left”, “front”, “back”, “upper”, “lower”, “above”, “below”, “top”, or “bottom” and variations thereof herein is for the purpose of description and should not be regarded as limiting. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Unless otherwise specified or limited, phrases similar to “at least one of A, B, and C,” “one or more of A, B, and C,” etc., are meant to indicate A, or B, or C, or any combination of A, B, and/or C, including combinations with multiple or single instances of A, B, and/or C.

In some non-limiting examples, aspects of the present disclosure, including computerized implementations of methods, can be implemented as a system, method, apparatus, or article of manufacture using standard programming or engineering techniques to produce software, firmware, hardware, or any combination thereof to control a processor device, a computer (e.g., a processor device operatively coupled to a memory), or another electronically operated controller to implement aspects detailed herein. Accordingly, for example, non-limiting examples of the invention can be implemented as a set of instructions, tangibly embodied on a non-transitory computer-readable media, such that a processor device can implement the instructions based upon reading the instructions from the computer-readable media. Some non-limiting examples of the invention can include (or utilize) a device such as an automation device, a special purpose or general purpose computer including various computer hardware, software, firmware, and so on, consistent with the discussion below.

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally, it should be appreciated that a carrier wave can be employed to carry computer-readable electronic data such as those used in transmitting and receiving electronic mail or in accessing a network such as the Internet or a local area network (LAN). Those skilled in the art will recognize many modifications may be made to these configurations without departing from the scope or spirit of the claimed subject matter.

Certain operations of methods according to the invention, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular non-limiting examples of the invention. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” etc. are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

As used herein, the term, “controller” and “processor” and “computer” include any device capable of executing a computer program, or any device that includes logic gates configured to execute the described functionality. For example, this may include a processor, a microcontroller, a field-programmable gate array, a programmable logic controller, etc. As another example, these terms may include one or more processors and memories and/or one or more programmable hardware elements, such as any of types of processors, CPUs, microcontrollers, digital signal processors, or other devices capable of executing software instructions.

As used herein, the term “fluid” includes both liquids, and gases as typically used, with the property of being able to readily change its shape when acted upon by a force, and to take the shape of a container that it is situated in. Thus, flow of the fluid can include other materials that flow with the fluid, such as debris, particulates, aerosols, etc. Additionally, fluid can include more than one fluid. For example, the fluid can include air, and another fluid can include a liquid suspended in the air, such as suspended droplets. In some cases, the allowing one fluid to flow does not mean that another fluid is allowed to flow. For example, fluid ports as described herein can allow gases to readily flow through, but can prevent liquids suspended in the gasses (e.g., pathogen laden aerosols) from passing through (e.g., if for example, a filter is present).

1. A system for enclosing and separating one of a patient from personnel while the personnel perform a procedure on the patient, or a laboratory sample from a technician while studying the laboratory sample, the system comprising:
a barrier configured to form a three-dimensional (3D) structure to define an interior volume and an exterior space and to mitigate fluid movement from the interior volume and into the exterior space thereby mitigating droplet, particulate, and aerosol movement in the flow path of the fluid from the interior volume and into the exterior space;
a plurality of passages extending through the barrier to provide access from the exterior space and into the interior volume to perform the procedure on the patient or perform the study on the laboratory sample arranged in the interior volume,
the barrier is configured to be formed in the 3D structure to arrange a portion of the patient or the laboratory sample in the interior volume, and
the barrier is positioned external to the patient if the personnel perform a procedure on the patient.

2. The system of claim 1, wherein a free end of the barrier includes a flange that extends along and engages with a portion of a patient or the laboratory sample to enclose the patient or the laboratory sample or a structure.

3. The system of claim 2, wherein the flange includes a coupling layer that couples to the portion of a patient or the laboratory sample or the structure.

4. The system of claim 3, wherein the flange has a first surface and a second surface, and
wherein the coupling layer is coupled to the first surface of the barrier.

5. The system of claim 4, wherein the coupling layer includes an adhesive.

6. The system of claim 1, further comprising a support structure that maintains at least a portion of the 3D structure.

7. The system of claim 6, wherein the support structure includes a scaffold that is coupled to an interior volume of the barrier, coupled to the exterior surface of the barrier, or integrated within the barrier.

8. The system of claim 7, wherein the scaffold includes a plurality of support beams.

9. The system of claim 8, wherein a first support beam within the plurality of support beams is curved.

10. The system of claim 8, wherein two adjacent support beams within the plurality of support beams are separated along an axial axis of the 3D structure.

11. The system of claim 10, wherein a given support beam within the plurality of support beams is coupled to the two adjacent support beams.

12. The system of claim 11, wherein the given support beam includes a resilient portion, the resilient portion configured to bend to allow the given support beam to be folded or shaped.

13. The system of claim 8, further comprising a sleeve coupled to at least one of the interior surface and the exterior surface of the barrier, the sleeve configured to receive a support beam.

14. The system of claim 7, wherein the barrier includes:
a central region;
a first region with a first free end extending from the central region, the first region having a first edge; and
a second region with a second free end extending from the central region, the second region having a second edge, and
wherein the first edge of the first region is configured to be coupled to the second edge of the second region to join the first region to the second region, and
wherein coupling of the first edge to the second edge provides an adjoined edge of the barrier that mitigates fluid movement from the interior volume and into the exterior space along the adjoined edge thereby mitigating droplet, particulate, and aerosol movement from the interior volume and interior the exterior space along the adjoined edge.

15. The system of claim 14, wherein the first edge of the first region includes a strip that extends along a portion of the first edge.

16. The system of claim 15, wherein the strip includes and an adhesive layer configured to be secured to a surface of the second region.

17. The system of claim 15, wherein the strip is removable coupled to a surface of the second region.

18. The system of claim 17, wherein the strip includes at least one of a hook and a loop fastener, and the surface of the second region includes the other of the at least one of the hook and the loop fastener.

19. The system of claim 14, wherein the first edge of the first region includes a first sleeve that extends along a portion of the first edge, and
wherein the second edge of the second region includes a second sleeve that extends along a portion of the first edge, and
wherein the scaffolding includes a support beam that is received though the first sleeve and though the second sleeve to couple the first region to the second region.

20. The system of claim 19, wherein the first sleeve is positioned above the second sleeve.

21-243. (canceled)
